(19) United States
(12) Patent Application Publication
(10) Pub. No.: US 2010/0166839 A1
(43) Pub. Date: Jul. 1, 2010

(54) NON PEPTIDIC RENIN INHIBITORS NITRODERIVATIVES

(75) Inventors: Nicoletta Almirante, Milano (IT); Stefano Biondi, Milano (IT); Ennio Ongini, Milano (IT)

Correspondence Address: ARENT FOX LLP, 1050 CONNECTICUT AVENUE, N.W., SUITE 400, WASHINGTON, DC, 20036, US

(73) Assignee: Nicox S.A, Sophia Antipolis (FR)

(21) Appl. No.: 12/278,674

(22) PCT Filed: Mar. 1, 2007
(86) PCT No.: PCT/EP2007/051933

§ 371 Date: Aug. 7, 2008

Related U.S. Application Data
(60) Provisional application No. 60/782,551, filed on Mar. 16, 2006.

(51) Int. Cl. A61K 31/547; C07D 401/12; A61K 31/46; A61K 31/504; A61K 31/5386; A61K 31/444; C07D 211/78; A61K 31/4418; C07D 221/22; A61K 31/443; A61K 31/616; A61K 9/127; A61P 9/00; A61P 9/10; A61P 13/12; A61P 27/06; A61P 1/16; A61P 15/10; A61P 5/42; A61P 11/00; A61P 17/00; A61P 25/22

(52) U.S. Cl. 424/450; 546/125; 514/304; 544/349; 514/249; 544/105; 514/230.5; 544/47; 514/224.2; 546/16; 514/278; 546/261; 514/335; 546/316; 514/355; 546/132; 546/283.7; 514/338; 514/161

(57) ABSTRACT

Non-peptidic renin inhibitors nitroderivatives of general formula (I):

A¹-(Xₐ—ONO₂)ⱼ (I)

having wider pharmacological activity and enhanced tolerability. They can be employed for treating or preventing cardiovascular, renal and chronic liver diseases, inflammatory processes and metabolic syndrome.

The present invention relates to nitroderivatives of non-peptidic renin inhibitors, pharmaceutical compositions containing them and their use for the treatment or prophylaxis of cardiovascular, renal and chronic liver diseases, inflammatory processes and metabolic syndrome.

Renin is a proteolytic enzyme which is predominantly released into the blood from the kidney. It cleaves its natural substrate, angiotensinogen, releasing decapeptide, angiotensin I. This is in turn cleaved by converting enzyme (ACE) in the lung, kidney and other tissues to the octapeptide angiotensin II, which has an effect on blood pressure. Angiotensin II raises blood pressure both directly by causing arteriolar constriction and indirectly by stimulating release of the sodium-retaining hormone aldosterone from the adrenal gland causing a rise in extracellular fluid volume.

The activity of the renin-angiotensin system can be manipulated pharmacologically by the inhibition of the activity of renin (renin inhibitors), or by the inhibition of the angiotensin converting enzyme (ACE inhibitors) or by blockade of angiotensin II receptors (angiotensin II receptor blockers).

Renin inhibitors have been developed as agents for control of hypertension, congestive heart failure, and hyperaldosteronism. Inefficient absorption, high first-pass metabolism and biliary excretion have constituted an obstacle to the clinical development of this group of drugs. The insufficient oral activity are due to their peptidomimetic character.

The need was felt to have available new renin inhibitors with good oral bioavailability and long duration of action. It has been so surprisingly found that nitroderivatives of renin inhibitors of a non-peptidic nature have a significantly improved overall profile as compared to the compounds above mentioned both in term of wider pharmacological activity and enhanced tolerability.

In particular, it has been recognized that the non-peptidic renin inhibitors nitroderivatives of the present invention exhibit a strong anti-inflammatory, antithrombotic and antiplatelet activity and can be furthermore employed for treating or preventing congestive heart failure, coronary diseases, left ventricular dysfunction and hypertrophy, cardiac fibrosis, myocardial ischemia, stroke, atherosclerosis, restenosis post angioplasty, renal ischemia, renal failure, renal fibrosis, glomerulonephritis, renal colic, ocular and pulmonary hypertension, glaucoma, systemic hypertension, diabetic complications such as nephropathy, vasculopathy and neuropathy, peripheral vascular diseases, liver fibrosis, portal hypertension, metabolic syndromes, erectile dysfunction, complications after vascular or cardiac surgery, complications of treatment with immunosuppressive agents after organ transplantation, hyperaldosteronism, lung fibrosis, scleroderma, anxiety, cognitive disorders.

Object of the present invention are, therefore, non-peptidic renin inhibitors nitroderivatives of general formula (I) and pharmaceutically acceptable salts or stereoisomers thereof:

A¹-(Xₐ—ONO₂)ⱼ (I)

wherein:

j is an integer equal to 1, 2, or 3;

A¹ is selected from the group consisting of formula (Ia), (Ib), (Ic), (Id), (Ie), (If), (Ig), (Ih), (Ii), (Ij), (Ik) and (Il):

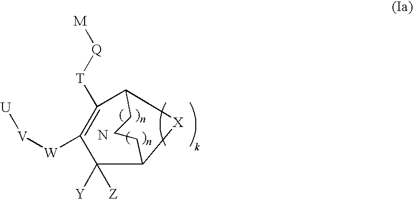

wherein:

—N₁— is a nitrogen atom bound to one group —Xₐ—ONO₂;

Y and Z represent independently from each other hydrogen (H); —F; -Me group; or Y and Z may together form a cyclopropyl ring; in case k represents the integer 1, Y and Z both represent hydrogen;

X represents —(CH₂)ₘN₁(L)(CH₂)ₘ—; —CH₂CH(K)CH₂—; —CH₂CH₂—; —CH₂OCH₂—; —CH₂SCH₂—; —CH₂SOCH₂—; —CH₂SO₂CH₂—; —CON(L)CO—; —CON(L)CHR⁶—; —CHR⁶—N(L)CO—;

W represents a six-membered, non benzofused, phenyl or heteroaryl ring, substituted by V in position 3 or 4;

V represents a bond; —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-(CH₂)₂—B—CH2-; —CH₂-A-(CH₂)₂—B—; —(CH₂)₃-A-(CH₂)₂—; —(CH₂)₄-A-CH₂—; -A-(CH₂)₂—B—(CH2)2-; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(OH₃)₂—CH₂—O—; —O—CH₂—CH(CH₃)—O—; —O—CH(CH₃)—CH₂—O—; —O—CH₂—C(CH₂—CH₂)—O—; —O—C(CH₂—CH₂)—CH₂—O—;

A and B represent —O—; —S—; —SO—; —SO₂—;

U represents aryl; heteroaryl;

T represent —CONR¹; —(CH₂)ₚOCO—; —(CH₂)ₚN(R¹)CO—; —(CH₂)ₚN(R¹)SO₂—; —COO—;

Q represents lower alkylene; lower alkenylene;

M represents aryl-O(CH₂)ᵥR⁵; heteroaryl-O(CH₂)ᵥR⁵; aryl-O(CH₂)ᵥO(CH₂)_{w}R⁵; heteroaryl-(CH₂)ᵥO(CH₂)_{w}R⁵; aryl-OCH₂CH(R⁷)CH₂R⁵; heteroaryl-OCH₂CH(R⁷)CH₂R⁵; wherein heteroayl means preferably a lower alkyl substituted pyridyl;

L represent —R³; —COR³; —CO₂R³; —CONR²R³; —SO₂R³; —SO₂NR²R³; —COCH(aryl)₂;

K represents hydrogen; —CH₂OR³; —CH₂NR²R³; —CH₂NR²COR³; —CH₂NR²SO₂R³; —CO₂R³; —CH₂OCONR²R³; —CONR²R³; —CH₂NR²COR²R³; —CH₂SO₂NR²R³; —CH₂SR³; —CH₂SOR³; —CH₂SO₂R³;

R₁ represents hydrogen, lower alkyl, lower alkenyl, lower alkynyl, cycloalkyl, aryl, cycloalkyl-lower alkyl;

R² and R^{2′} independently represent hydrogen, lower alkyl; lower alkenyl, cycloalkyl, cycloalkyl-lower alkyl;

R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di- or trisubstituted with hydroxy, —OCOR², —COOR², lower alkoxy, cyano, —CONR²R^{2′}, —CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH, lower alkoxy, —OCOR², —CO₂R², NR²R^{2′}, OCONR²R^{2′}, NCONR²R^{2′}, cyano, —CONR²R^{2′}, SO₃H, —SONR²R^{2′}; —CO-morpholin-4-yl, —CO-((4-loweralkyl)piperazin-1-yl), —NH(NH)NH₂, NR⁴R^{4′} with the proviso that a carbon atom is attached at the most to one heteroatom in case this carbon atom is sp³-hybridized; when R⁵ is —O—, it is bound to one group —Xₐ—ONO₂;

R⁶ represents hydrogen, lower alkyl; lower alkoxy; whereby these groups may be unsubstituted or monosubstituted with hydroxy, —CONH₂, —COOH, imidazolyl, —NH₂, —CN, —NH(NH)NH₂;

R⁷ represents —O—; —OH, OR², OCOR², OCOOR²; or R⁶ and R⁵ form together with the carbon atoms to which they are attached a 1,3-dioxolane ring which is substituted in position 2 with R² and R^{2′}; or R⁶ and R⁵ form together with the carbon atoms to which they are attached a 1,3-dioxolan-2-one ring; when R⁷ is —O—, it is bound to one group —Xₐ—ONO₂;

k represents the integer 0 or 1;

m and n represent the integer 0 or 1 with the proviso that in case m represents the integer 1, n is the integer 0; in case n represents the integer 1, m is the integer 0; in case k represents the integer 0, n represents the integer 0; in case X does not represent —(CH₂)ₘ—N(L)-(CH₂)ₘ—, n represents the integer 0;

p, t and v independently represent the integer 1,2,3 or 4;

r represents the integer 1,2,3,4,5 or 6;

s represents the integer 1,2,3,4 or 5;

u represents the integer 1,2 or 3;

w represents the integer 1 or 2.

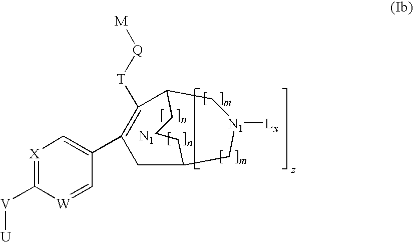

wherein

—N₁— is a nitrogen atom bound to one group —Xₐ—ONO₂;

X and W represent a nitrogen atom or a —CH— group;

V represents —(CH₂)ᵣ; -A-(CH₂)ₛ; —CH₂-A-(CH₂)ₜ-; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;

A and B represent independently —O—; —S—; —SO—; —SO₂—;

U represents aryl; heteroaryl;

T represents —CONR¹—; —(CH₂)ₚOCO—; —(CH₂)ₚN(R¹)CO—; —(CH₂)ₚN(R¹)SO₂—; —CO₂—;

Q represents lower alkylene or alkenylene;

M represents aryl-O(CH₂)ᵥR⁷—; heteroaryl-O(CH₂)ᵥR⁷; aryl-O(CH₂)ᵥO(CH₂)_{w}R⁷; heteroaryl-(CH₂)ᵥO(CH₂)_{w}R⁷; aryl-OCH₂CH(R⁶)CH₂R⁵—; heteroaryl-OCH₂CH(R⁶)CH₂R⁵—;

L when x is 1 represents —R³; —COR³; —COOR³; —CONR²R³; SO₂R³; —SO₂NR²R³; COCH(Aryl)₂;

R¹ represents hydrogen; lower alkyl; lower alkenyl; or lower alkynyl; cycloalkyl; aryl; cycloalkyl-lower alkyl;

R² and R^{2′} independently represent hydrogen, lower alkyl; lower alkenyl; cycloalkyl; cycloalkyl-lower alkyl;

R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di- or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH; lower alkoxy; —OCOR²; —COOR²; —NR²R^{2′}; —OCONR²R^{2′}; —NCONR²R^{2′}; cyano; —CONR²R^{2′}; —SO₃H; —SONR²R^{2′}; —CO-morpholin-4-yl; —CO-((4-loweralkyl)piperazin-1-yl); —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;

R⁶ represents —O—; —OH; OR²; OCOR²; OCOOR²; or R⁶ and R⁵ form together with the carbon atoms to which they are attached a 1,3-dioxolane ring which is substituted in position 2 with R² and R^{2′}; or R⁶ and R⁵ form together with the carbon atoms to which they are attached a 1,3-dioxolan-2-one ring; when R⁶ is —O—, it is bound to one group —Xₐ—ONO₂;

R⁷ represents lower alkoxy;

m and n represent the integer 0 or 1, with the proviso that in case m represents the integer 1, n is the integer 0, and in case n represents the integer 1, m is the integer 0;

p, t, and v represent the integer 1,2, 3 or 4;

r represents the integer 3,4,5 or 6;

s represents the integer 2, 3, 4 or 5;

u represents the integer 1, 2 or 3;

w represents the integer 1 or 2;

x and z represent the integer 0 or 1.

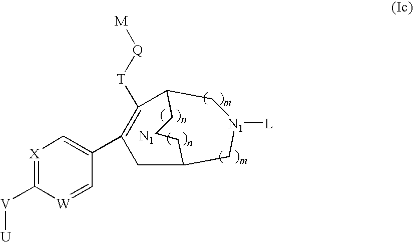

wherein:

—N₁— is a nitrogen atom bound to one group —Xₐ—ONO₂;

X and W represent a nitrogen atom or a —CH— group;

V represents —(CH₂)ᵣ; -A-(CH₂)ₛ; —CH₂-A-(CH₂)ₜ; —(CH₂)ₛ-A-; —(CH₂)₂-A-(CH₂)ᵤ—; -A-(CH₂)ᵥ—B—; —(CH₂)₃-A-CH₂; -A-CH₂CH₂—B—CH₂—; —CH₂-A-CH₂CH₂—B—; —(CH₂)₃-A-CH₂—CH₂—; —(CH₂)₄-A-CH₂—; —A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;

A and B represent independently —O—; —S—; —SO—; —SO₂—;

U represents aryl; heteroaryl;

T represents —CONR¹—; —(CH₂)ₚOCO—; —(CH₂)ₚN(R¹)CO—; —(CH₂)ₚN(R¹)SO₂—; —CO₂—;

Q represents lower alkylene; lower alkenylene;

M represents hydrogen; cycloalkyl; aryl; heterocyclyl; heteroaryl;

L represents —R³; —COR³; —COOR³; —CONR²R³; —SO₂R³; —SO₂NR²R³; COCH(Aryl)₂;

R¹ represents hydrogen; lower alkyl; lower alkenyl; lower alkynyl; cycloalkyl; aryl; cycloalkyl-lower alkyl;

R² and R^{2′} independently represent hydrogen, lower alkyl; lower alkenyl; cycloalkyl; cycloalkyl-lower alkyl;

R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di- or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

m and n represent the integer 0 or 1, with the proviso that in case m represents the integer 1, n is the integer 0, and in case n represents the integer 1, m is the integer 0;

p and t represent the integer 1,2, 3 or 4;

r represents the integer 3,4,5 or 6;

s represents the integer 2, 3, 4 or 5;

u represents the integer 1, 2 or 3;

v represents the integer 2, 3 or 4.

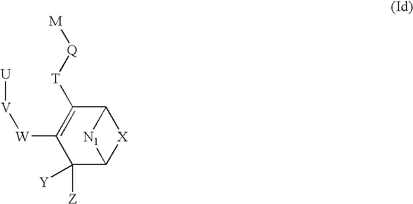

wherein:

—N₁— is a nitrogen atom bound to one group —Xₐ—ONO₂;

Y, Z represent independently hydrogen, F, or o methyl group; or Y and Z may together form a cyclopropyl ring;

X represents —CH₂CH(K)CH₂—, —CH₂CH₂—, —CH₂OCH₂—; —CH₂SCH₂—, —CH₂SOCH₂—, —CH₂SO₂CH₂—, —CO—NL-CHR⁶—; —CHR⁶—NL-CO—;

W represents a six membered non benzofused phenyl, or heteroaryl ring substituted by V in position 3 or 4;

V represents a bond, represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(OCH₃)₂—CH₂—O—; —O—CH_{2 —C(CH}₃)₂—CH₂—O—; —O—CH₂—C(CH₃)₂—O—; —O—C(CH₃)₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;

A and B independently represent —O—, —S—, —SO—, —SO₂—;

U represents aryl, heteroaryl;

T represents —CONR¹—; —(CH₂)ₚOCO—; —(CH₂)ₚN(R¹)CO—; (CH₂)ₚN(R¹)SO₂; —CO₂—;

Q represents lower alkylene, lower alkenylene;

M represents aryl-O(CH₂)ᵥR⁵, heteroaryl-O(CH₂)ᵥR⁵, aryl-OCH₂CH₂O(CH₂)_{w}R⁵; heteroaryl-OCH₂CH₂O(CH₂)_{w}R⁵;

L represents —R³; —COR³; —COOR³; —CONR²R³; —SO₂R³; —SO₂NR²R³; COCH(Aryl)₂;

K represents hydrogen; —CH₂OR³; —CH₂NR²R³; —CH₂NR²COR³; —CH₂NR²SO₂R³; —CO₂R³; —CH₂OCONR²R³; —CONR²R³; —CH₂NR²COR²R³; —CH₂SO₂NR²R³; —CH₂SR³; —CH₂SOR³; —CH₂SO₂R³;

R¹ represents hydrogen, lower alkyl, lower alkenyl, lower alkynyl, cycloalkyl, aryl, cycloalkyl-lower alkyl;

R² and R^{2′} independently represent hydrogen, lower alkyl, lower alkenyl, cycloalkyl, cycloalkyl-lower alkyl;

R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di- or trisubstituted with hydroxy, —OCOR², —COOR², lower alkoxy, cyano, —CONR²R^{2′}, —CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH, —OCOR², —CO₂R², —NR²R^{2′}, —OCONR²R^{2′}, —NCONR²R^{2′}, cyano, —CONR²R^{2′}, SO₃H, —SONR²R^{2′}; —CO-morpholin-4-yl, —CO-((4-loweralkyl)piperazin-1-yl), —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;

R⁶ represents hydrogen, lower alkyl; lower alkoxy; whereby these groups may be unsubstituted or monosubstituted with hydroxy, —CONH₂, —COOH, imidazolyl, —NH₂, —CN, —NH(NH)NH₂;

p and t independently represent the integer 1, 2, 3 or 4;

r represents the integer 1, 2, 3, 4, 5 or 6;

s represents the integer 1, 2, 3, 4 or 5;

u represents the integer 1, 2 or 3;

v represents the integer 2, 3 or 4;

w represents the integer 1 or 2.

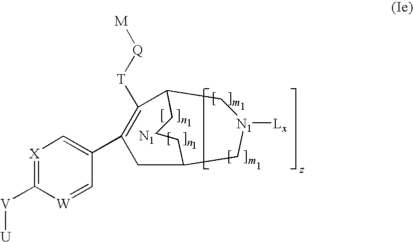

wherein:

—N₁— is a nitrogen atom bound to one group —Xₐ—ONO₂;

X and W represent a nitrogen atom or a —CH— group;

V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;

A and B represent independently —O—; —S—; —SO—; —SO₂—;

U represents aryl; heteroaryl;

T represents —CONR¹—; —(CH₂)ₚOCO—; —(CH₂)ₚN(R¹)CO—; —(CH₂)ₚN(R¹)SO₂—; —CO₂—;

Q represents lower alkylene or alkenylene;

M represents aryl-O(CH₂)ᵥR⁵—; heteroaryl-O(CH₂)ᵥR⁵; aryl-O(CH₂)₂O(CH₂)_{w}R⁵; heteroaryl-(CH₂)₂O(CH₂)_{w}R⁵;

L represents —R³; —COR³; —COOR³; —CONR²R³; —SO₂R³; —SO₂NR²R³; COCH(Aryl)₂;

R¹ represents hydrogen; lower alkyl; lower alkenyl; or lower alkynyl; cycloalkyl; aryl; cycloalkyl-lower alkyl;

R² and R^{2′} independently represent hydrogen, lower alkyl; lower alkenyl; cycloalkyl; cycloalkyl-lower alkyl;

R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di- or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH; —OCOR²; —COOR²; —NR²R^{2′}; —OCONR²R^{2′}; —NCONR²R^{2′}; cyano; —CONR²R^{2′}; —SO₃H; —SONR²R^{2′}; —CO-morpholin-4-yl; —CO-((4-loweralkyl)piperazin-1-yl); —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;

m₁ and n₁ represent the integer 0 or 1, with the proviso that in case m₁ represents the integer 1, n₁ is the integer 0, and in case n₁ represents the integer 1, m₁ is the integer 0;

p, t, represent the integer 1, 2, 3 or 4;

r represents the integer 3, 4, 5 or 6;

s represents the integer 2, 3, 4 or 5;

u represents the integer 1, 2 or 3;

v represents the integer 2, 3 or 4;

w represents the integer 1 or 2;

x represents the integer o or 1;

z represents the integer 0 or 1; if z is the integer 0, n₁ is the integer 1.

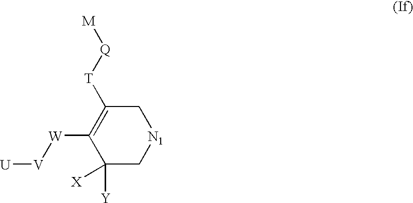

wherein:

—N₁— is a nitrogen atom bound to one group —Xₐ—ONO₂;

X and Y independently represent hydrogen, —F, or a methyl group; X and Y do not represent both hydrogen at the same time or X and Y may together form a cyclopropyl ring;

W represents a phenyl or heteroaryl ring, the heteroaryl ring being a six-membered and non-fused ring, the phenyl ring and the heteroaryl ring are substituted with V in position 3 or 4;

V represents —(CH₂)ᵣ; -A-(CH₂)ₛ; —CH₂-A-(CH₂)ₜ; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;

U represents aryl, heteroaryl;

A and B independently represent —O—, —S—, —SO—, —SO₂—;

T represents —CONR¹—; —(CH₂)ₚOCO—; —(CH₂)ₚN(R¹)CO—; —(CH₂)ₚN(R¹)SO₂—; —CO₂—; (CH₂)ₚOCO—NR¹; —(CH₂)ₚN(R²)CO—NR¹—

R¹ and R² independently represent hydrogen, lower alkyl, lower alkenyl, lower alkynyl, cycloalkyl, aryl-lower alkyl; heteroaryl-lower alkyl; cycloalkyl-lower alkyl;

Q represents lower alkylene, lower alkenylene;

M represents hydrogen; cycloalkyl; aryl; heterocyclyl or heteroaryl;

p and t independently represent the integer 1, 2, 3 or 4;

r represents the integer 3, 4, 5 or 6;

s represents the integer 2, 3, 4 or 5;

u represents the integer 1, 2 or 3;

v represents the integer 2, 3 or 4.

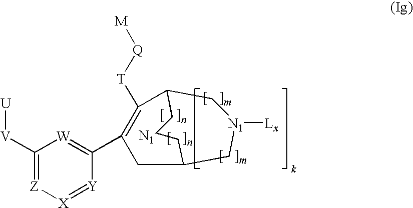

wherein:

—N₁— is a nitrogen atom bound to one group —Xₐ—ONO₂;

Z, Y, X and W represent independently a nitrogen atom, or a —CH— group; at least two of the Z, Y, X and W represent a —CH— group;

V represents a bond; —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂₋; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;

A and B represent independently —O—; —S—; —SO—; —SO₂—;

U represents aryl; heteroaryl;

T represents —CONR¹—; —(CH₂)ₚOCO—; —(CH₂)ₚN(R¹)CO—; —(CH₂)ₚN(R¹)SO₂—CO₂—;

Q represents lower alkylene or alkenylene;

M represents hydrogen; cycloalkyl; aryl; heteroaryl; heterocyclyl;

L when k is 1 represents —R³; —COR³; —COOR³; —CONR²R³; —SO₂R³; —SO₂NR²R³; COCH(Aryl)₂;

R¹ represents hydrogen; lower alkyl; lower alkenyl; or lower alkynyl; cycloalkyl; aryl; cycloalkyl-lower alkyl;

R² and R^{2′} independently represent hydrogen, lower alkyl; lower alkenyl; cycloalkyl; cycloalkyl-lower alkyl;

R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di- or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

k represents the integer 0 or 1;

m and n represent the integer 0 or 1, with the proviso that in case m represents the integer 1, n is the integer 0, and in case n represents the integer 1, m is the integer 0;

p and t represent the integer 1,2, 3 or 4;

r represents the integer 1, 2, 3, 4, 5 or 6;

s represents the integer 1, 2, 3, 4 or 5;

u represents the integer 1, 2 or 3;

v represents the integer 2, 3 or 4.

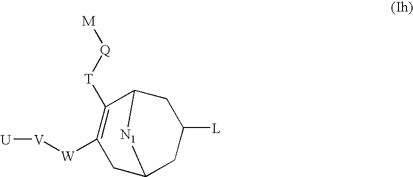

wherein:

—N₁— is a nitrogen atom bound to one group —Xₐ—ONO₂;

W is a six-membered non benzofused phenyl, or heteroaryl ring substituted by V in position 3 or 4;

V represents a bond, represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂₋; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;

A and B independently represent —O—, —S—, —SO—, —SO₂—;

U represents aryl, heteroaryl;

T represents —CONR¹—; —(CH₂)ₚOCO—; —(CH₂)ₚN(R¹)CO—; —(CH₂)ₚN(R¹)SO₂—; —CO₂—;

Q represents lower alkylene, lower alkenylene;

M represents hydrogen; cycloalkyl; aryl, hetrocyclyl; heteroaryl; aryl-O(CH₂)ᵥR⁵, heteroaryl-O(CH₂)ᵥR⁵, aryl-OCH₂CH₂O(CH₂)_{w}R⁵; heteroaryl-OCH₂CH₂O(CH₂)_{w}R⁵;

L represents hydrogen; —CH₂OR³; —CH₂NR²R³; —CH₂—NR²COR³; —CH₂NR²SO₂R³; —COOR³; —CH₂OCONR²R³; —CONR²R³; —CH₂NR²CONR²R³; —CH₂SO₂NR²R³; —CH₂SR³; —CH₂SOR³; —CH₂SO₂R³;

R¹ represents hydrogen; lower alkyl; lower alkenyl; lower alkynyl; cycloalkyl; aryl; cycloalkyl-lower alkyl;

R² and R^{2′} represent independently hydrogen; lower alkyl; lower alkenyl; cycloalkyl; cycloalkyl-lower alkyl;

R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di- or trisubstituted with hydroxy, —OCOR², —COOR², lower alkoxy, cyano, —CONR²R^{2′}, —CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH, —OCOR², —CO₂R², —NR²R^{2′}, —OCONR²R^{2′}, —NCONR²R^{2′}, cyano, —CONR²R^{2′}, SO₃H, —SONR²R^{2′}; —CO-morpholin-4-yl, —CO-((4-loweralkyl)piperazin-1-yl), —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;

p and t independently represent the integer 1,2,3 or 4;

r represents the integer 3, 4, 5 or 6;

s represents the integer 2, 3, 4 or 5;

u represents the integer 1, 2 or 3;

v represents the integer 2, 3 or 4;

w represents the integer 1 or 2.

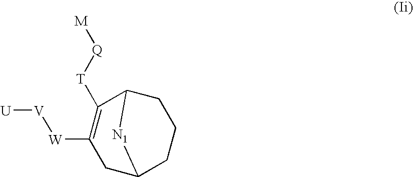

wherein:

—N₁— is a nitrogen atom bound to one group —Xₐ—ONO₂;

W is a six-membered non benzofused phenyl, or heteroaryl ring substituted by V in position 3 or 4;

V represents a bond, represents —(CH₂)ᵣ; -A-(CH₂)ₛ; —CH₂-A-(CH₂)ₜ—; —(CH₂)₅-A-; —(CH₂)₂-A-(CH₂)—-; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;

A and B independently represent —O—, —S—, —SO—, —SO₂—;

U represents aryl, heteroaryl;

T represents —CONR¹—; —(CH₂)ₚOCO—; —(CH₂)ₚN(R¹)CO—; —(CH₂)ₚN(R¹)SO₂—; —CO₂—;

Q represents lower alkylene, lower alkenylene;

M represents hydrogen; cycloalkyl; aryl; heterocyclyl; heteroaryl;

R¹ represents hydrogen, lower alkyl, lower alkenyl, lower alkynyl, cycloalkyl, aryl, cycloalkyl-lower alkyl;

p and t independently represent the integer 1,2,3 or 4;

r represents the integer 3,4,5 or 6;

s represents the integer 2,3,4 or 5;

u represents the integer 1,2 or 3;

v represents the integer 2,3 or 4.

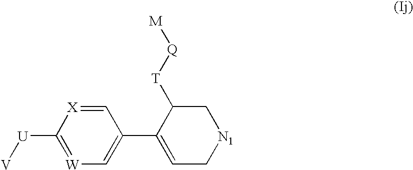

wherein:

—N₁— is a nitrogen atom bound to one group —Xₐ—ONO₂;

X and W represent a nitrogen atom or a —CH— group;

V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂₋; -A-CH₂CH₂—B—CH₂—; —CH₂-A-CH₂CH₂—B—; —(CH₂)₃-A-CH₂—CH₂—; —(CH₂)⁴-A-CH₂—; -A-CH₂CH₂—B—CH₂—CH₂—; —CH₂-A-CH₂CH₂—B—CH₂—; —CH₂-A-CH₂—; —CH₂-A-CH₂—CH₂—CH₂—B—; —CH₂—CH₂-A-CH₂CH₂B—;

A and B represent independently —O—; —S—; —S(O)—; —S(O)₂—;

U represents aryl; heteroaryl;

T represents —CONR¹—; —(CH₂)ₚOCO—; —(CH₂)ₚN(R¹)CO—; —(CH₂)ₚN (R¹)SO₂—; —CO₂—; —(CH₂)ₚOCONR¹—; —(CH₂)ₚN(R^{1′})CONR¹—;

Q represents lower alkylene; lower alkenylene;

M represents hydrogen, cycloalkyl; aryl; heterocyclyl; heteroaryl; aryl-O(CH₂)ᵥR²—; heteroaryl-O(CH₂)ᵥR²; aryl-O(CH₂)₂O(CH₂)_{w}R²; heteroaryl-(CH₂)₂O(CH₂)_{w}R²; aryl-OCH₂CH(R⁵)CH₂R²; heteroaryl-OCH₂CH(R⁵)CH₂R²

R¹ and R^{1′} independently represent hydrogen; lower alkyl; lower alkenyl; or lower alkynyl; cycloalkyl; aryl; cycloalkyl-lower alkyl;

R² represents —O—; —OH; lower alkoxy; —OCOR³; —COOR³; —NR³R^{3′}, —OCONR³R^{3′}; —NCONR³R^{3′}; cyano; —CONR³R³; —SO₃H; —CO-morpholin-4-yl; —CO—((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized; when R² is —O— can link to —Xₐ—ONO₂;

R³ and R^{3′} represent independently hydrogen; lower alkyl; lower alkenyl; cycloalkyl; cycloalkyl-lower alkyl;

R⁴ and R^{4′} independently represent hydrogen; lower alkyl; cycloalkyl; cycloalkyl-lower alkyl; hydroxy-lower alkyl; —COOR²; —CONH₂;

R⁵ represents —O—; —OH; —OR²; —OCOR²; —OCOOR²; or R⁵ and R² form together with the carbon atoms to which they are attached a 1,3 dioxalane ring which is substituted in position 2 with R³ and R^{3′}; or R⁵ and R² form together with the carbon atoms to which they are attached a 1,3 dioxola-2-one ring; when R⁵ is —O—, it is bound to one group —Xₐ—ONO₂;

p, t, represent the integer 1,2, 3 or 4;

r represents the integer 3,4,5 or 6;

s represents the integer 2, 3, 4 or 5;

u represents the integer 1, 2 or 3;

v represents the integer 2, 3 or 4;

w represents the integer 1 or 2.

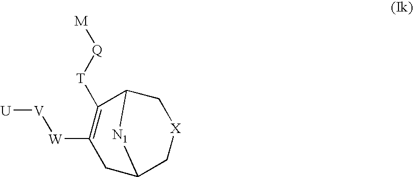

wherein:

—N₁— is a nitrogen atom bound to one group —Xₐ—ONO₂;

X represents —O—, —S—, —SO—, —SO₂—;

W is a six-membered non benzofused phenyl, or heteroaryl ring substituted by V in position 3 or 4;

V represents a bond, represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂₋; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;

A and B independently represent —O—, —S—, —SO—, —SO₂—;

U represents aryl, heteroaryl;

T represents —CONR¹—; —(CH₂)ₚOCO—; —(CH₂)ₚN(R¹)CO—; —(CH₂)ₚN(R¹) SO₂—; —CO₂—;

Q represents lower alkylene, lower alkenylene;

M represents hydrogen, cycloalkyl, aryl; heterocycyl; heteroaryl;

R¹ represents hydrogen, lower alkyl, lower alkenyl, lower alkynyl, cycloalkyl, aryl, cycloalkyl-lower alkyl;

p and t independently represent the integer 1,2,3 or 4;

r represents the integer 3,4,5 or 6;

s represents the integer 2,3,4 or 5;

u represents the integer 1,2 or 3;

v represents the integer 2,3 or 4.

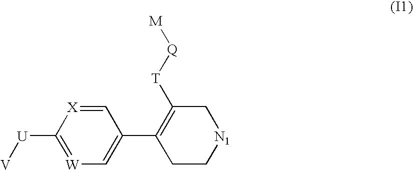

wherein:

—N₁— is a nitrogen atom bound to one group —Xₐ—ONO₂;

X and W represent independently a nitrogen atom or a —CH— group;

V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂₋; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;

A and B independently represent —O—, —S—, —SO—, —SO₂—;

U represents aryl, heteroaryl;

T represents —CONR¹—; —(CH₂)ₚOCO—; —(CH₂)N(R¹)CO—; —(CH₂)ₚN(R¹)SO₂—; —CO₂—; —(CH₂)ₚOCONR¹—; —(CH₂)ₚN(R^{1′})CONR¹—;

Q represents lower alkylene, lower alkenylene;

M represents hydrogen; cycloalkyl; aryl; heterocycyl; heteroaryl;

R¹ and R^{1′} represent independently hydrogen; lower alkyl; lower alkenyl; lower alkynyl; cycloalkyl; aryl; cycloalkyl-lower alkyl;

p and t independently represent the integer 1,2,3 or 4;

r represents the integer 3,4,5 or 6;

s represents the integer 2,3,4 or 5;

u represents the integer 1,2 or 3;

v represents the integer 2,3 or 4.

Xₐ is equal to —X_{b}—Yₐ— wherein X_{b} is —CO— or —COO—;

Yₐ is a bivalent radical having the following meaning:

a)

straight or branched C₁-C₂₀ alkylene, preferably C₁-C₁₀, being optionally substituted with one or more of the substituents selected from the group consisting of: halogen atoms, hydroxy, —ONO₂ or Tₐ, wherein Tₐ is —OC(O)(C₁-C₁₀ alkyl)-ONO₂ or —O(C₁-C₁₀alkyl)-ONO₂;

cycloalkylene with 5 to 7 carbon atoms into cycloalkylene ring, the ring being optionally substituted with side chains T_{b}, wherein T_{b} is straight or branched alkyl with from 1 to 10 carbon atoms, preferably CH₃;

b)

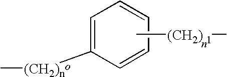

c)

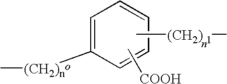

wherein n⁰ is an integer from 0 to 20, and n¹ is an integer from 1 to 20;

d)

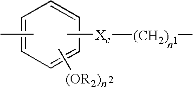

wherein:

n¹ is as defined above and n² is an integer from 0 to 2;

X_{c}═—OCO— or —COO— and R₂ is H or CH₃;

e)

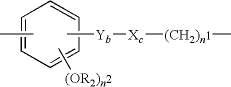

wherein:

n¹, n², R₂ and X_{c} are as defined above;

Y_{b} is —CH₂—CH₂— or —CH═CH—(CH₂)ₙ²—;

f)

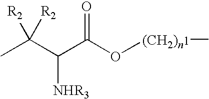

wherein:

n¹ and R₂ are as defined above, R₃ is H or —COCH₃;

with the proviso that when Yₐ is selected from the bivalent radicals mentioned under b)-f), the —ONO₂ group is linked to a —(CH₂)ₙ¹ group;

g)

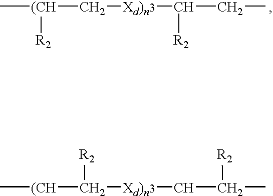

wherein X_{d} is —O— or —S—, n³ is an integer from 1 to 6, preferably from 1 to 4, R₂ is as defined above;

h)

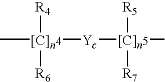

wherein:

n⁴ is an integer from 0 to 10;

n⁵ is an integer from 1 to 10;

R₄, R₅, R₆, R₇ are the same or different, and are H or straight or branched C₁-C₄ alkyl, preferably R₄, R₅, R₆, R₇ are H;

wherein the —ONO₂ group is linked to

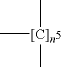

wherein n⁵ is as defined above;

Y_{c} is an heterocyclic saturated, unsaturated or aromatic 5 or 6 members ring, containing one or more heteroatoms selected from nitrogen, oxygen, sulfur, and is selected from the group consisting in:

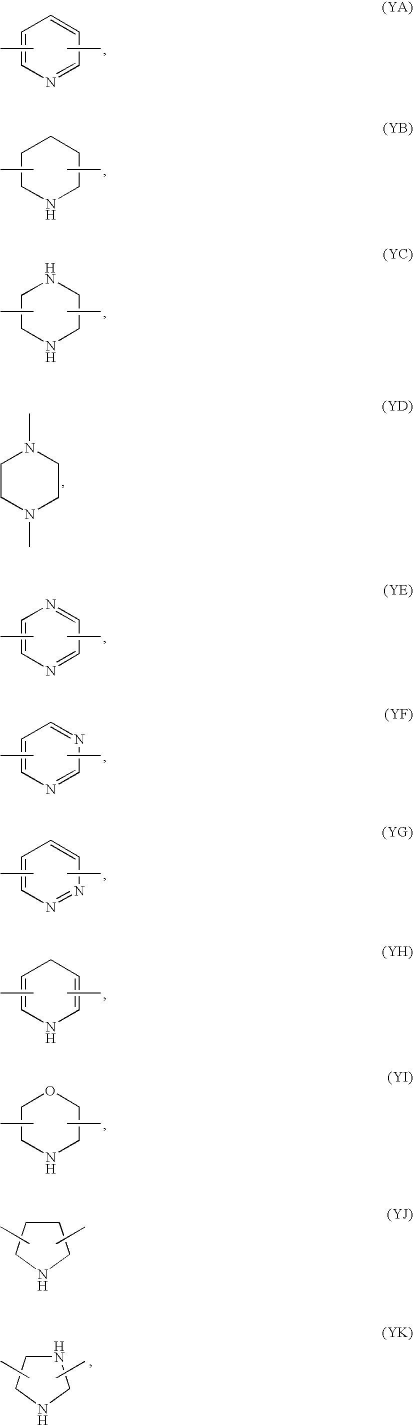

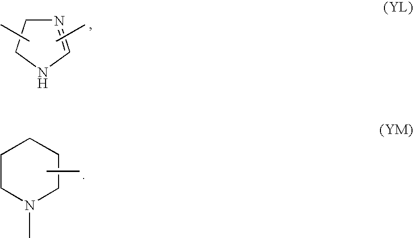

The term “C₁-C₂₀ alkylene” as used herein refers to branched or straight chain C₁-C₂₀ hydrocarbon, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight chain alkyl groups comprising one to ten carbon atoms, including methyl, ethyl, n-propyl, isopropyl, n-butyl, isobutyl, t-butyl, pentyl, hexyl, octyl and the like.

The term “cycloalkylene” as used herein refers to ring having from 5 to 7 carbon atoms including, but not limited to, cyclopentylene, cyclohexylene optionally substituted with side chains such as straight or branched (C₁-C₁₀)-alkyl, preferably CH₃.

The term “heterocyclic” as used herein refers to saturated, unsaturated or aromatic 5 or 6 members ring, containing one or more heteroatoms selected from nitrogen, oxygen, sulphur, such as for example pyridine, pyrazine, pyrimidine, pyrrolidine, morpholine, imidazole and the like.

Another aspect of the present invention provides the use of the compounds of formula (I) in combination with at least a compound used to treat cardiovascular disease selected from the group consisting of: aldosterone antagonists, angiotensin II receptor blockers, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin. Also is contemplated the combination with nitrosated compounds of the above reported compounds.

Suitable aldosterone antagonists, angiotensin II receptor blockers, ACE inhibitors, HMGCoA reductase inhibitors, beta-adrenergic blockers, alpha-adrenergic antagonists, calcium channel blockers, potassium activators, diuretics, vasodilators and antithrombotics are described in the literature such as The Merck Index (13^{th} edition).

Suitable nitrosated compounds are disclosed in WO 98/21193, WO 97/16405, WO 98/09948, WO 2004/105754, WO 2004/106300, WO 2004/110432, WO 2005/011646, WO 2005/053685, WO 2005/054218.

The administration of the compounds above reported can be carried out simultaneously or successively.

The present invention also provides pharmaceutical kits comprising one or more containers filled with one or more of the compounds and/or compositions of the present invention and one or more of the compounds used to treat cardiovascular diseases reported above.

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile, tetrahydrofuran with the corresponding organic or inorganic acids.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

Preferred compounds are those of formula (I) wherein Yₐ has the following meaning:

a)

straight or branched C₁-C₁₀ alkylene;

b)

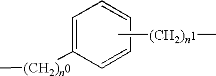

wherein n⁰ is 0 or 1, n¹ is 1;

with the proviso that the —ONO₂ group is linked to —(CH₂)ₙ¹ group;

g)

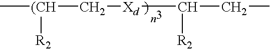

wherein X_{d} is —O— or —S—, n³ is 1 and R₂ is H;

The following are preferred compounds according to the present invention:

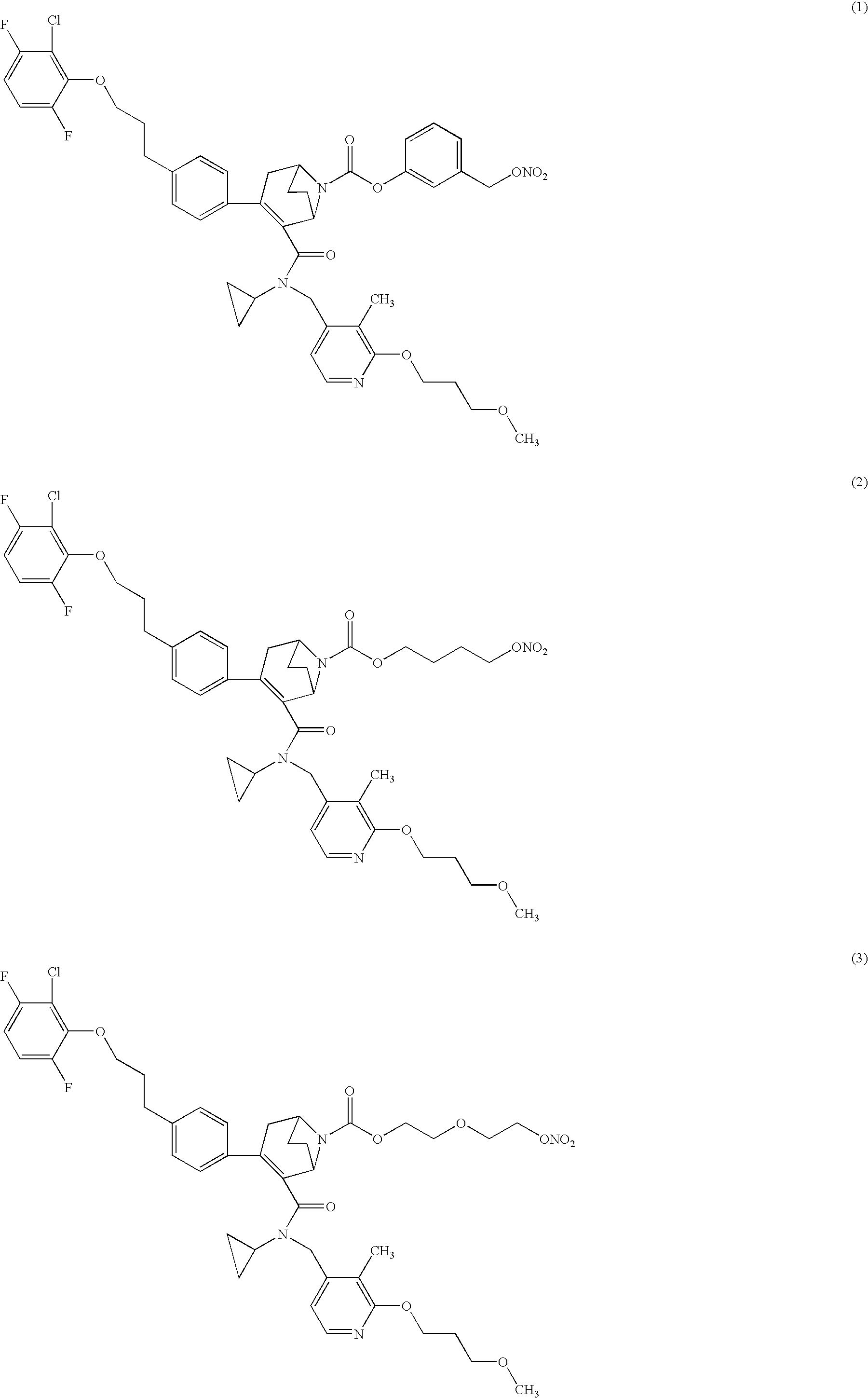

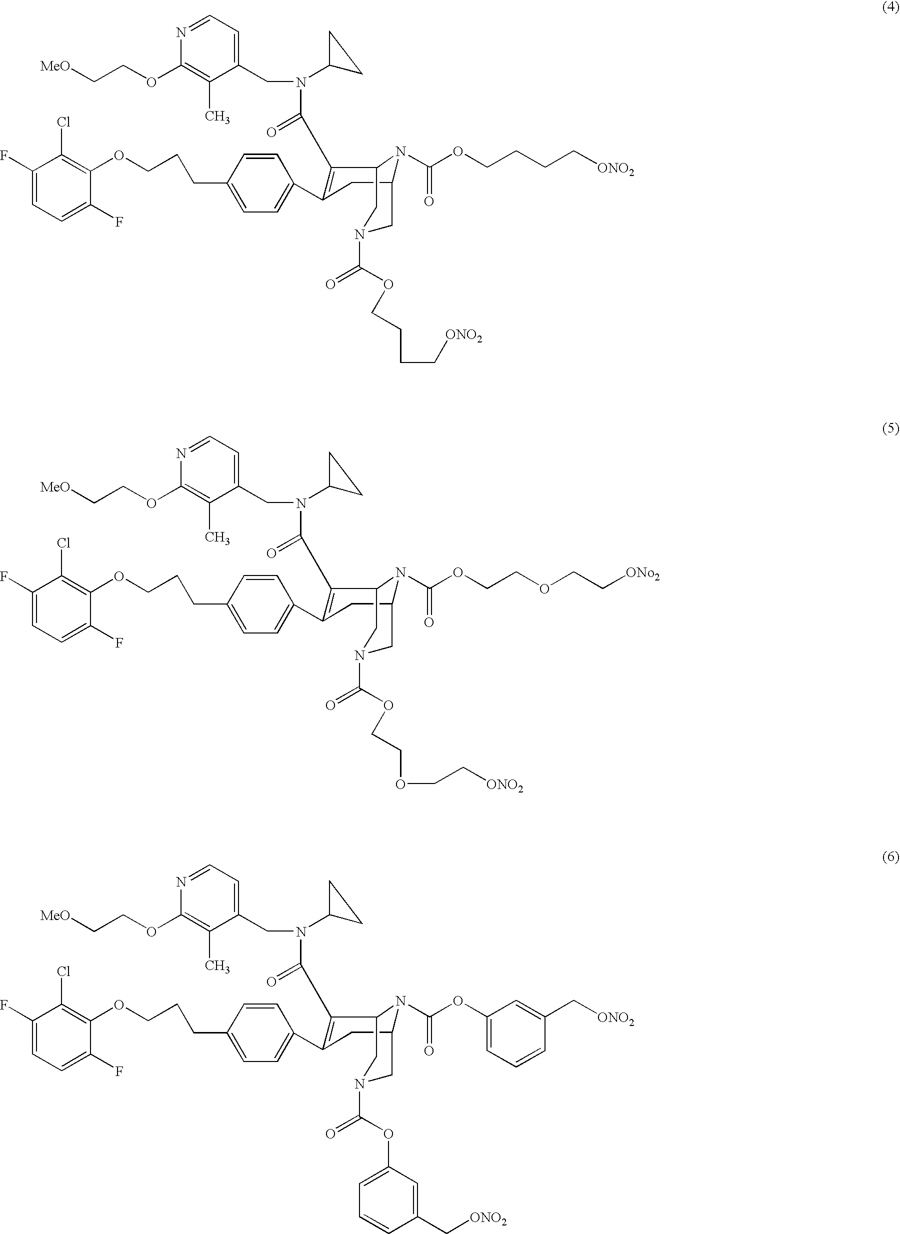

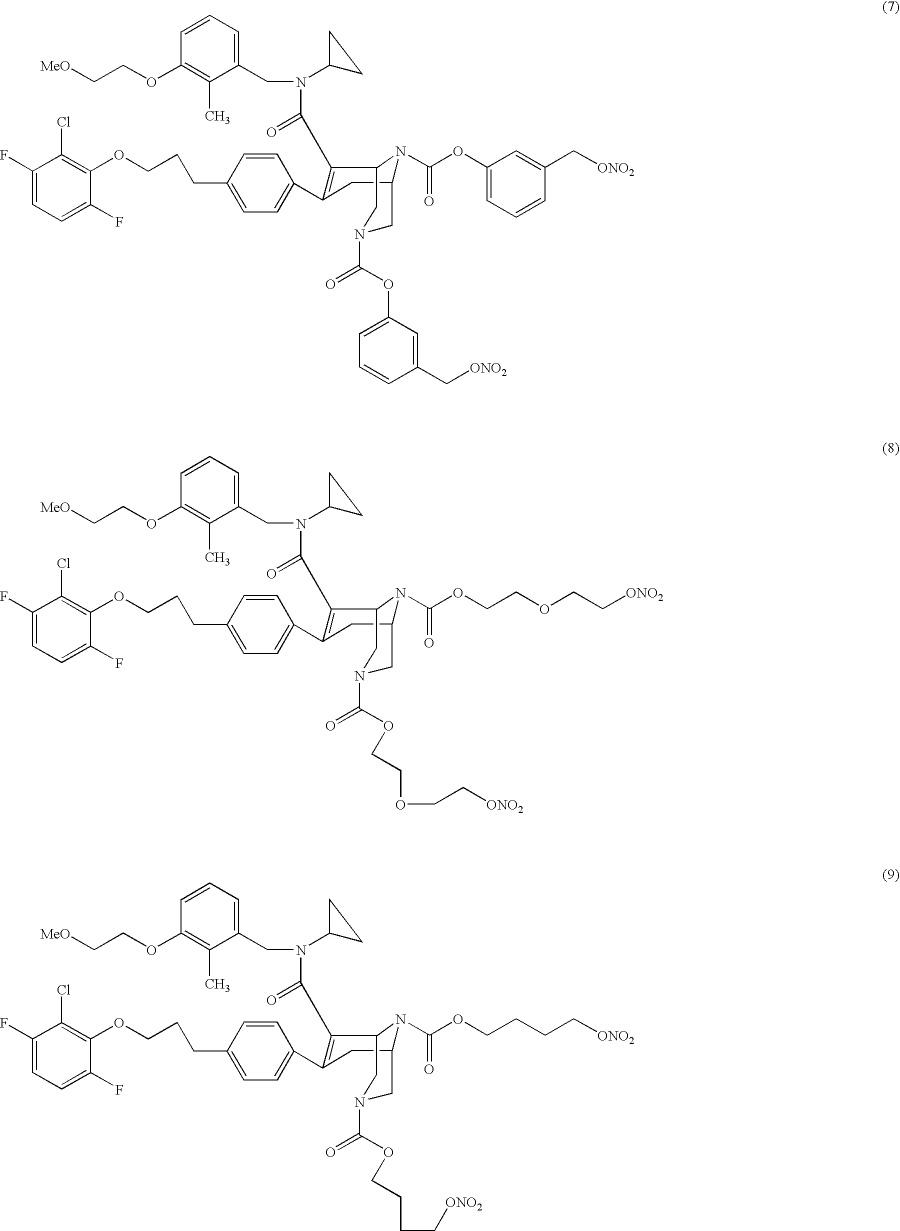

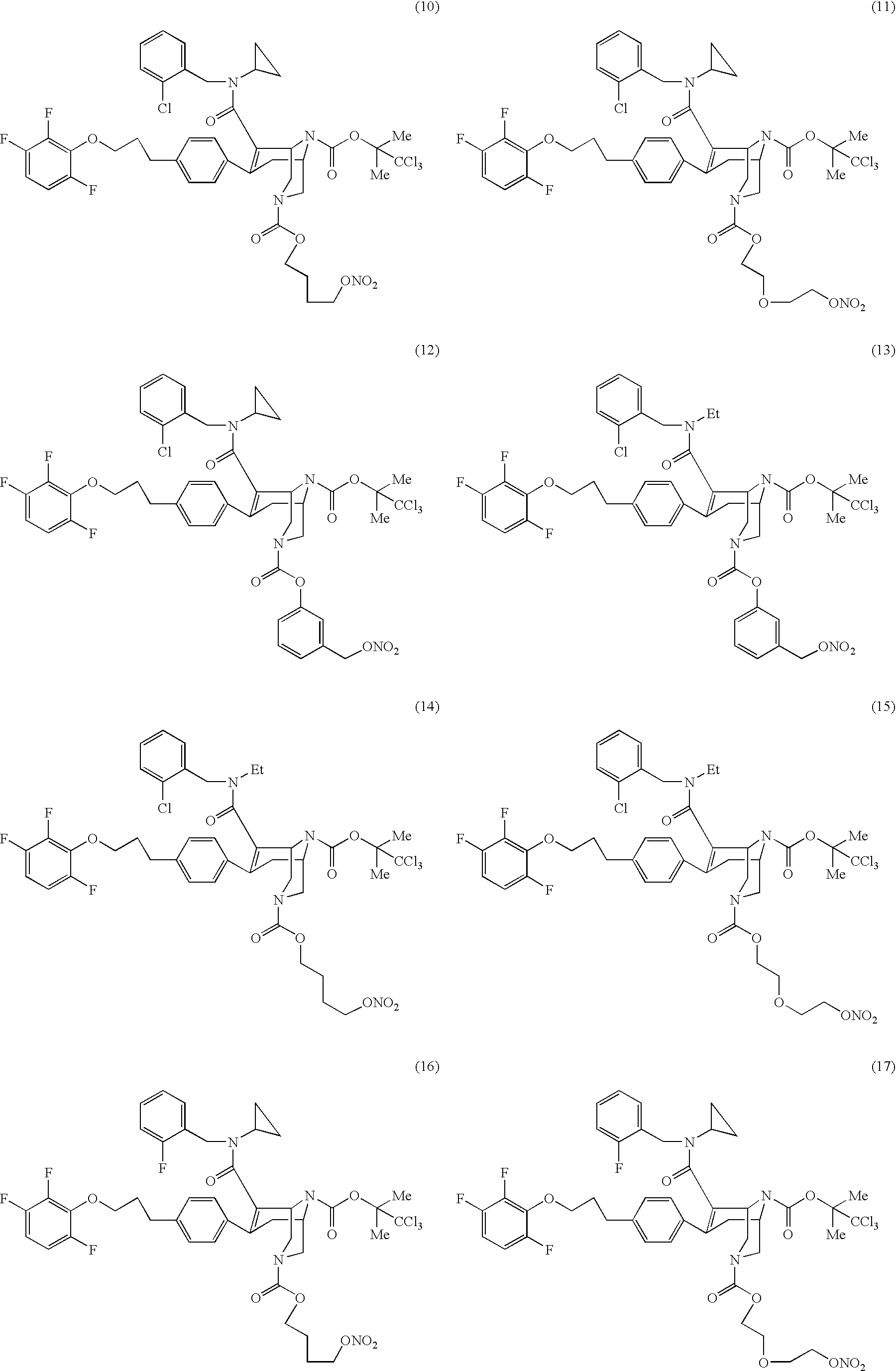

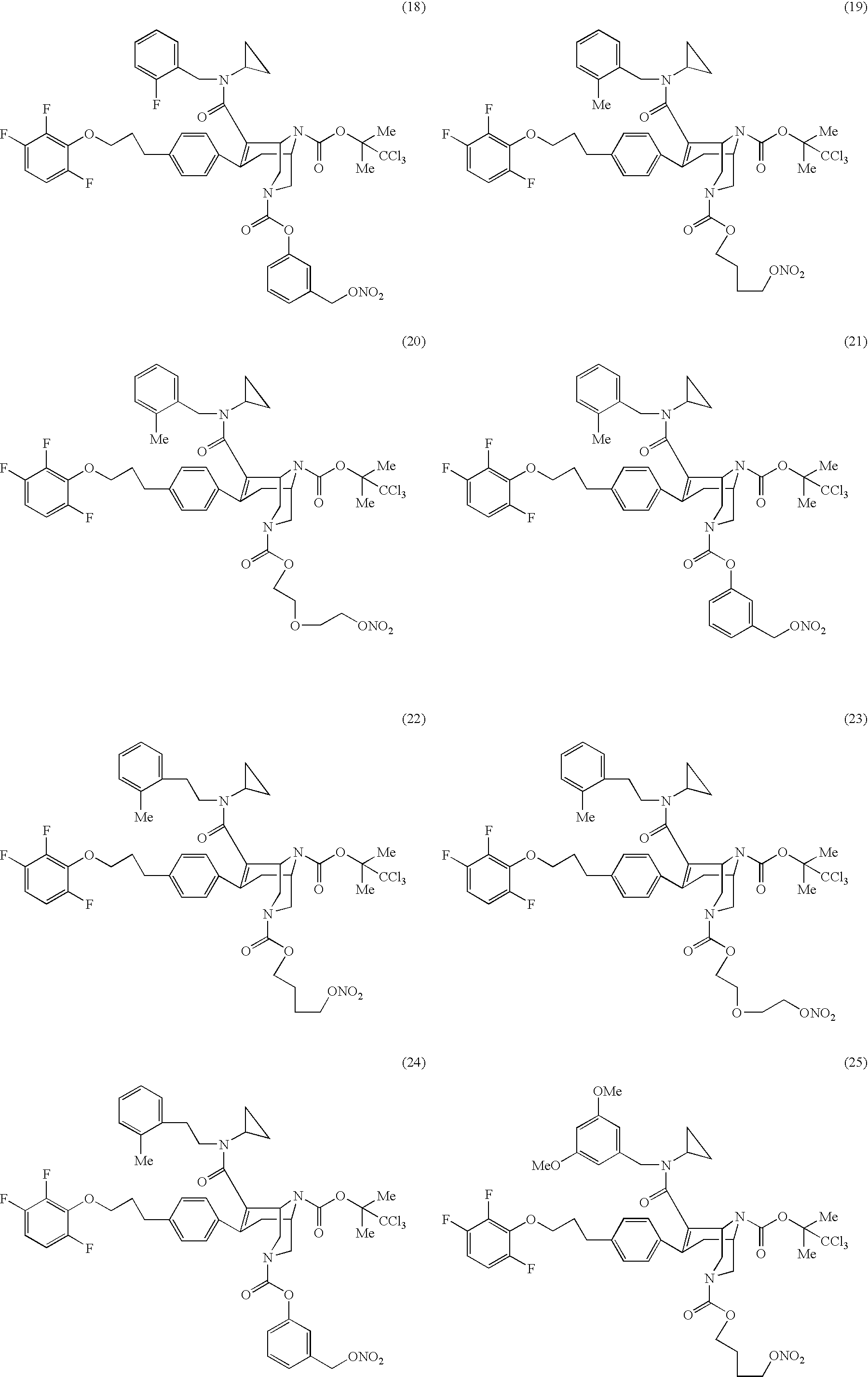

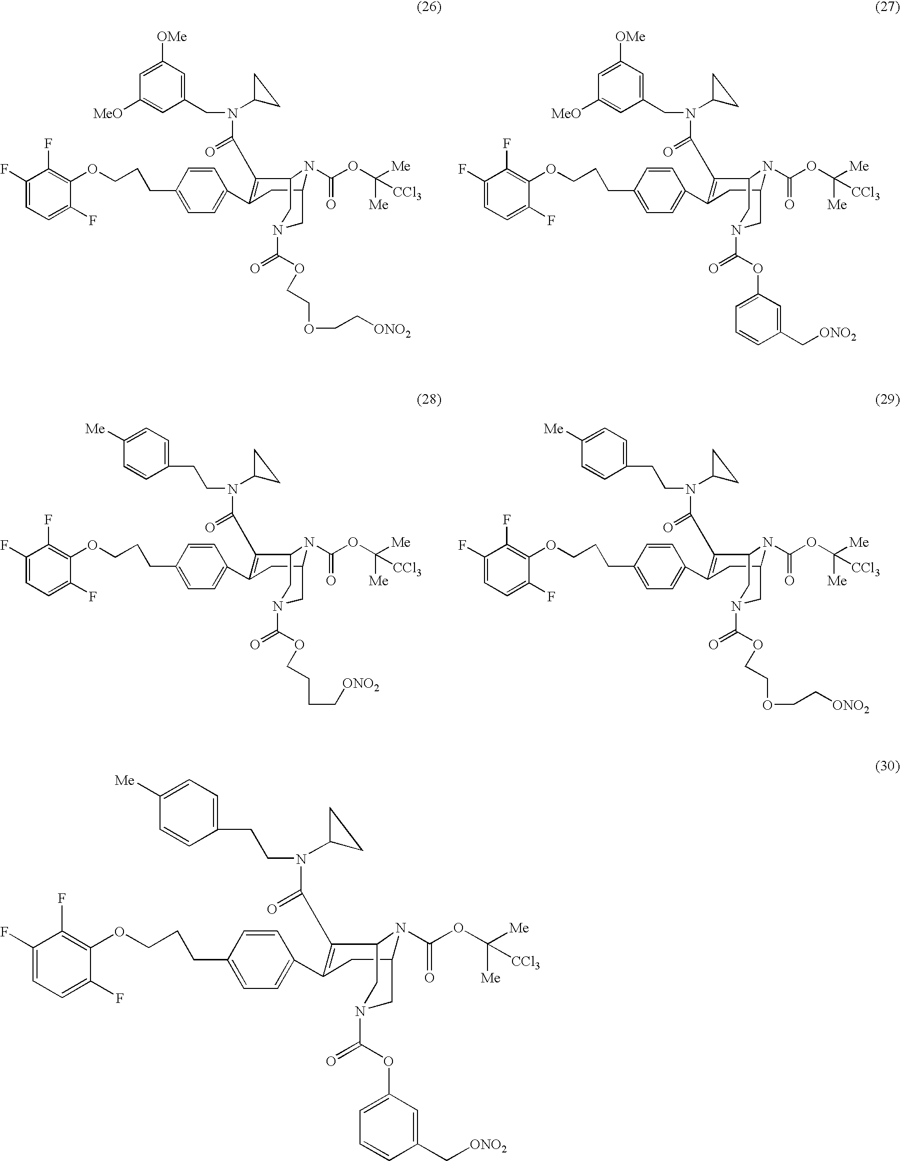

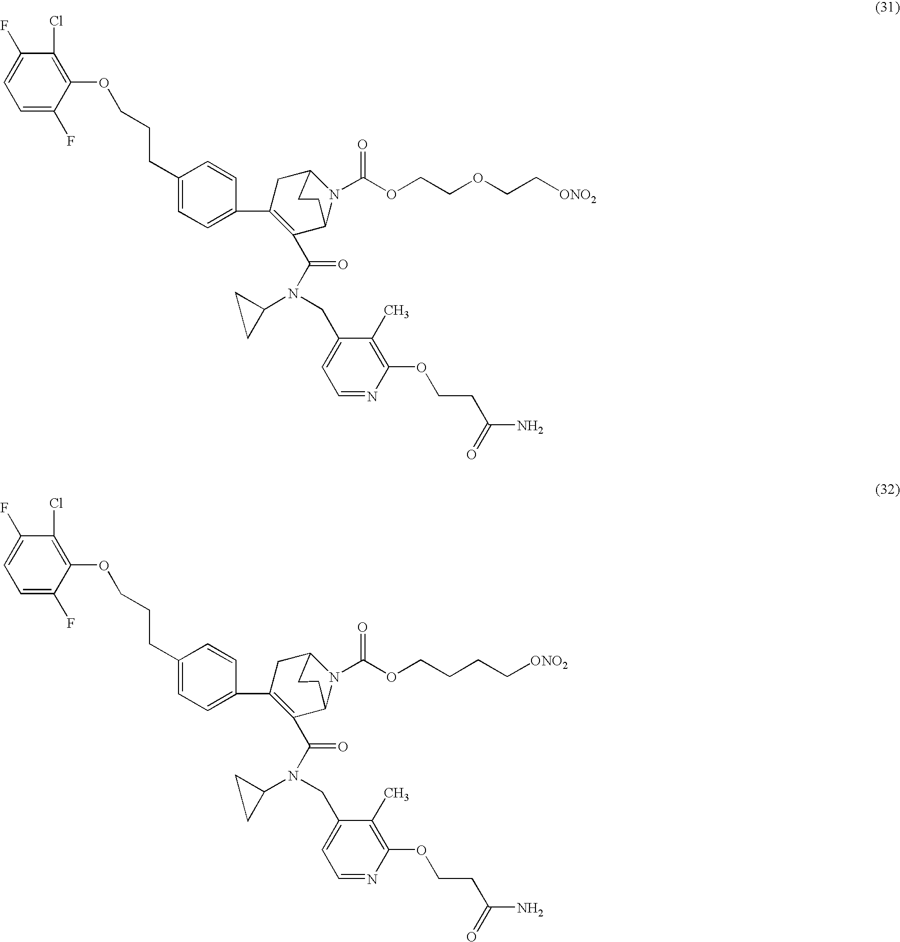

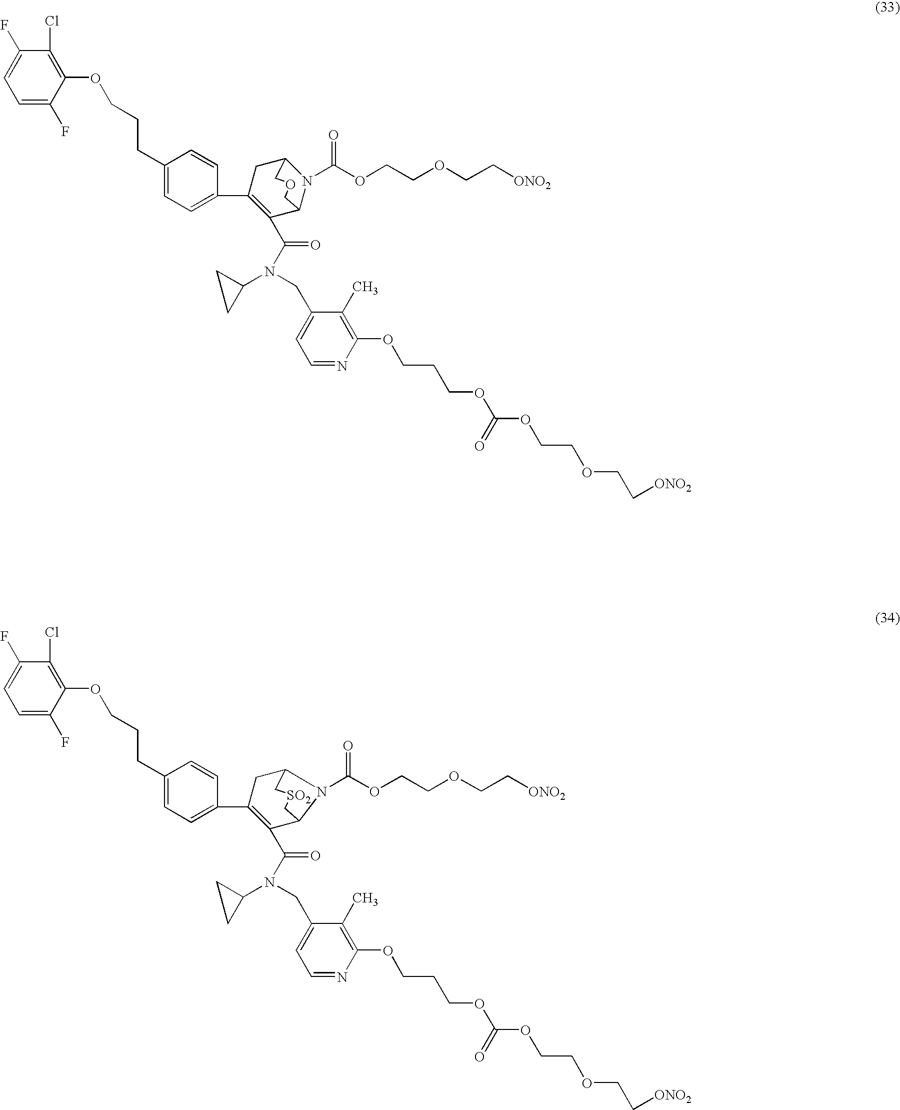

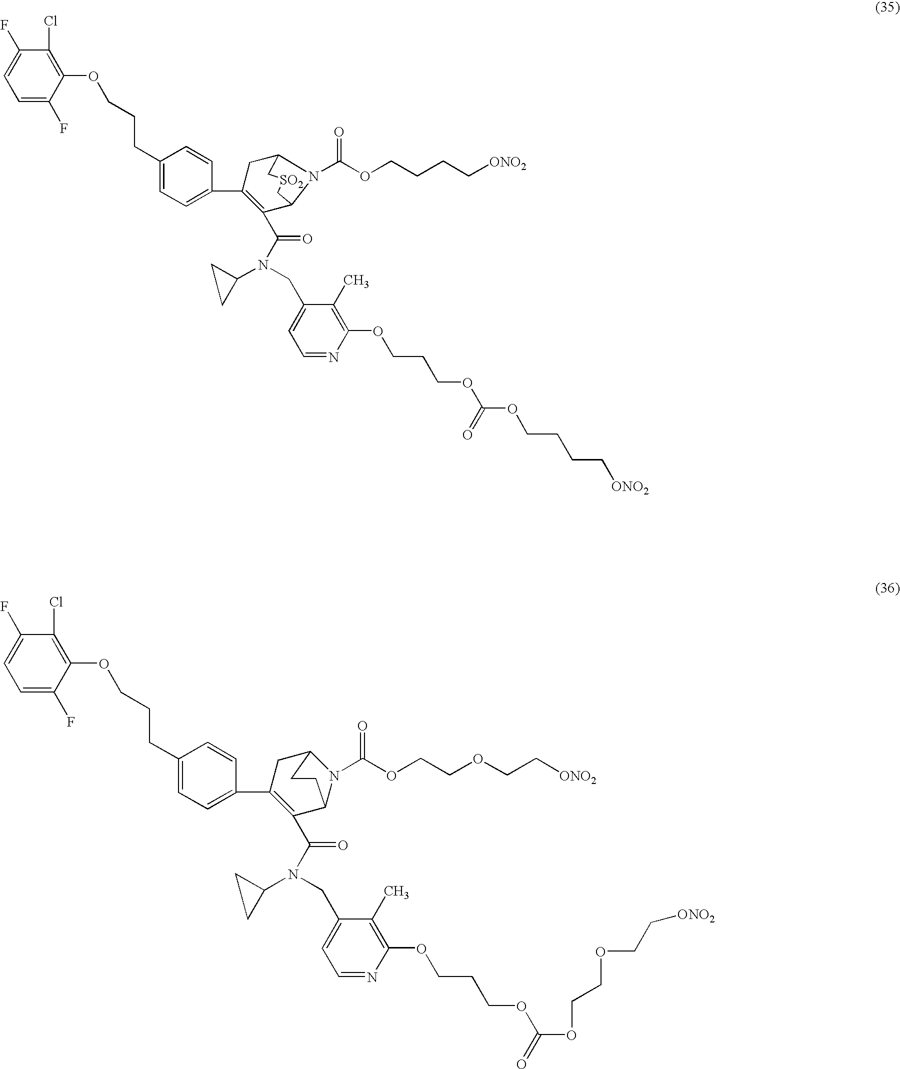

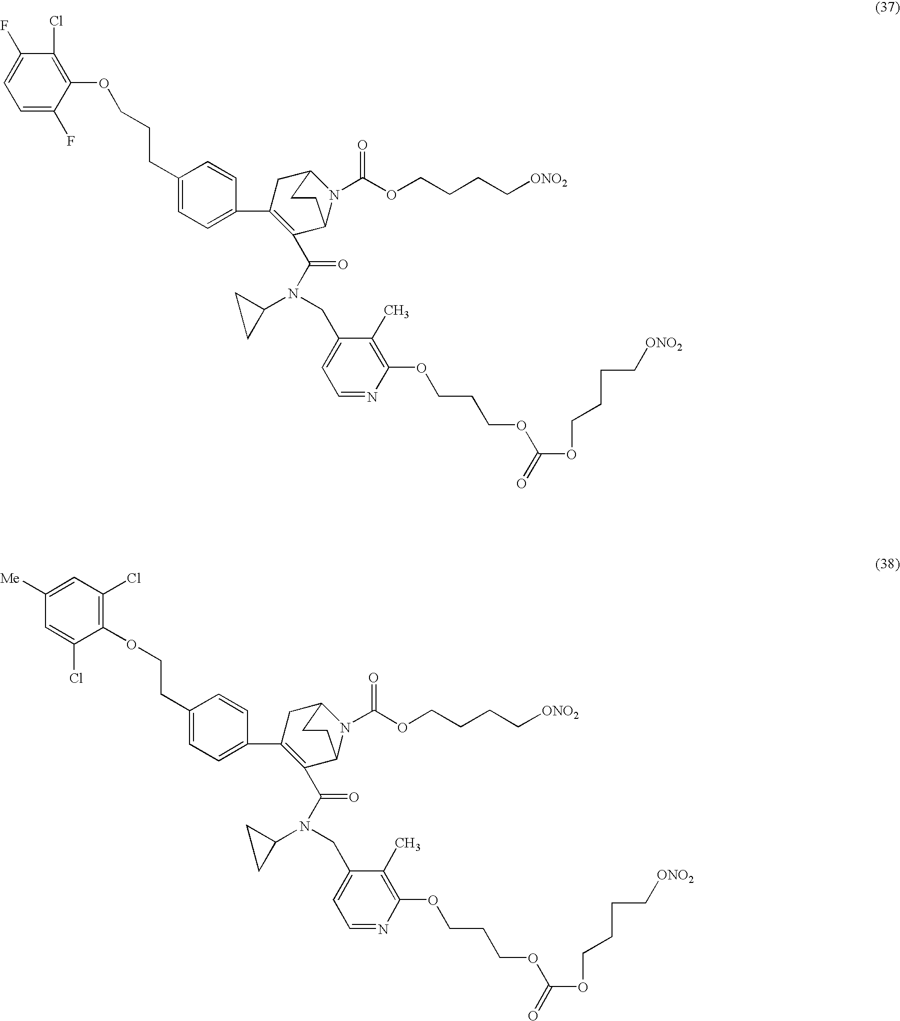

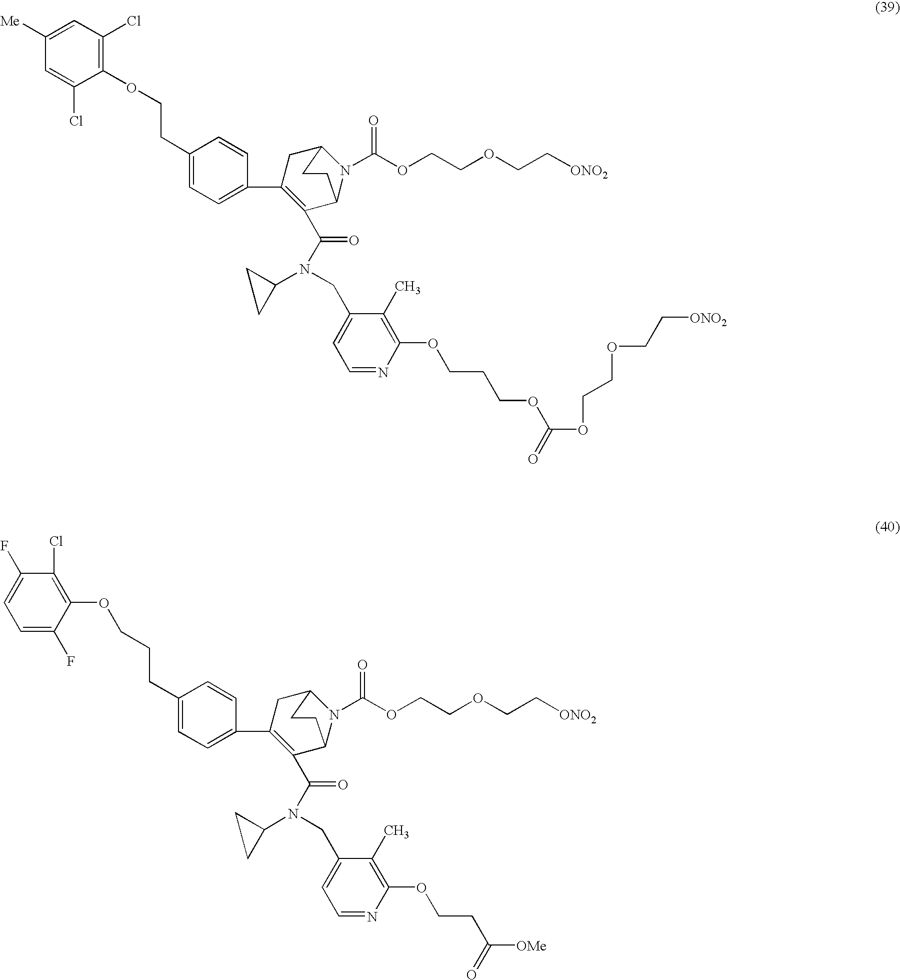

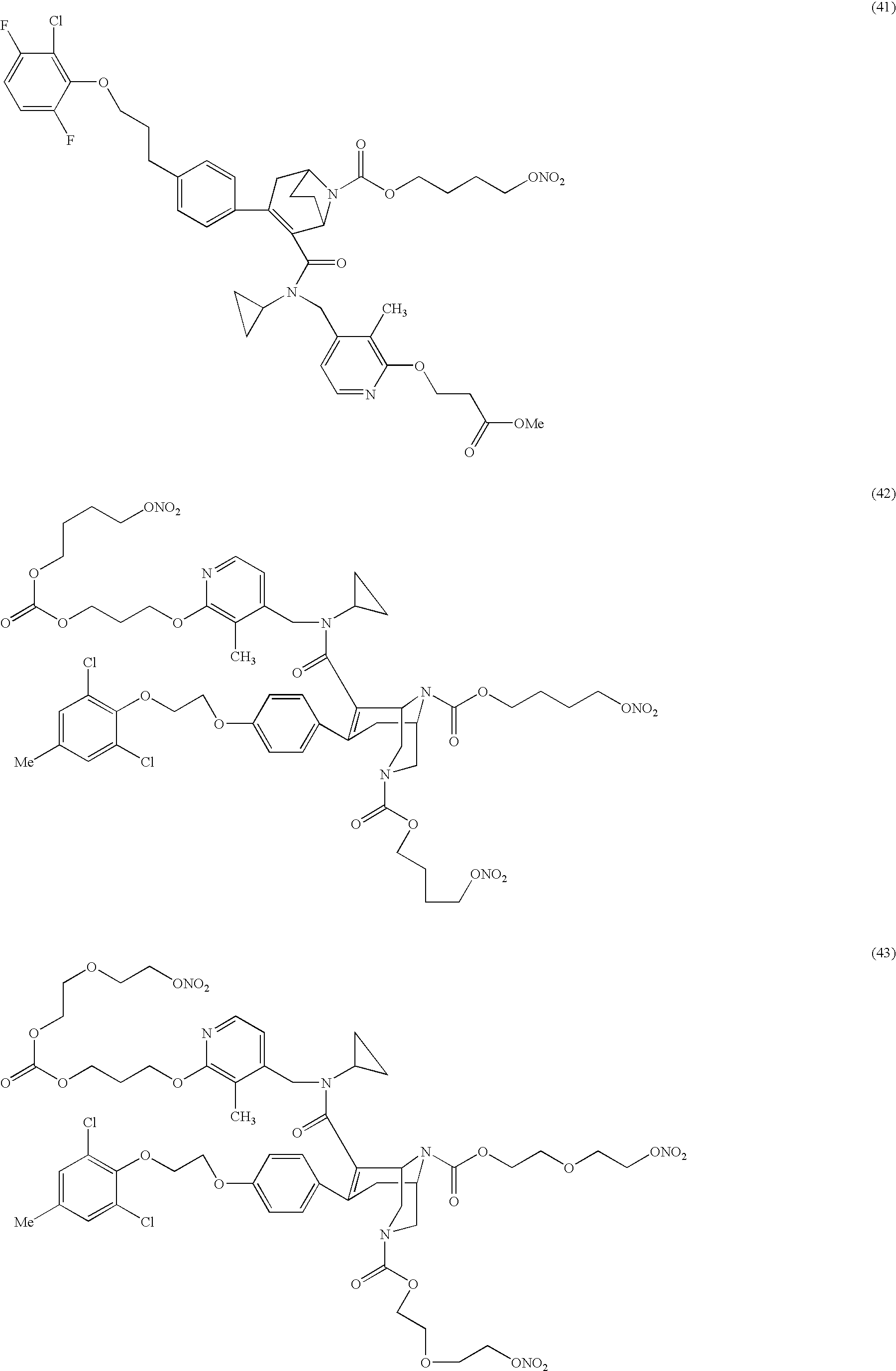

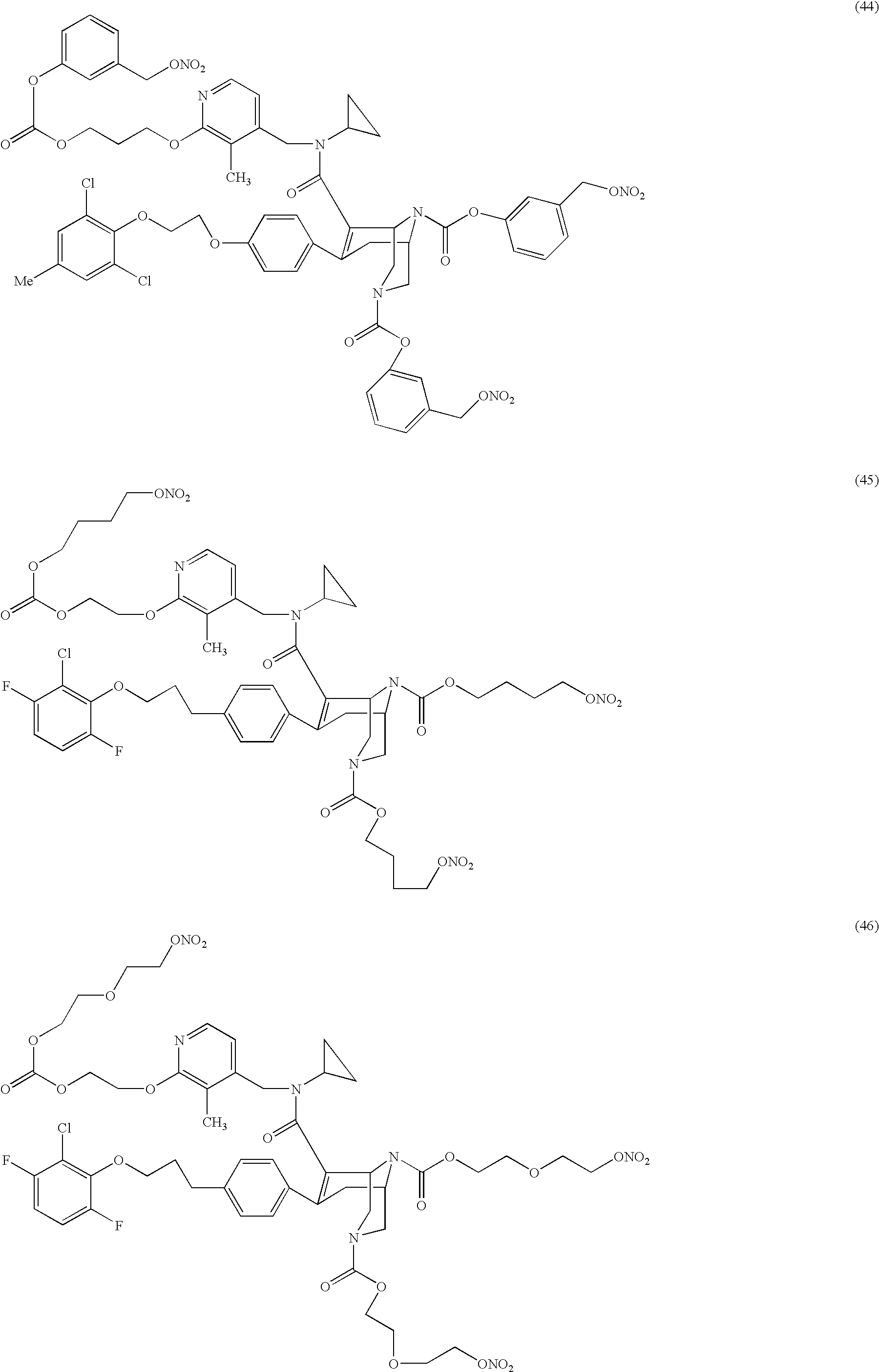

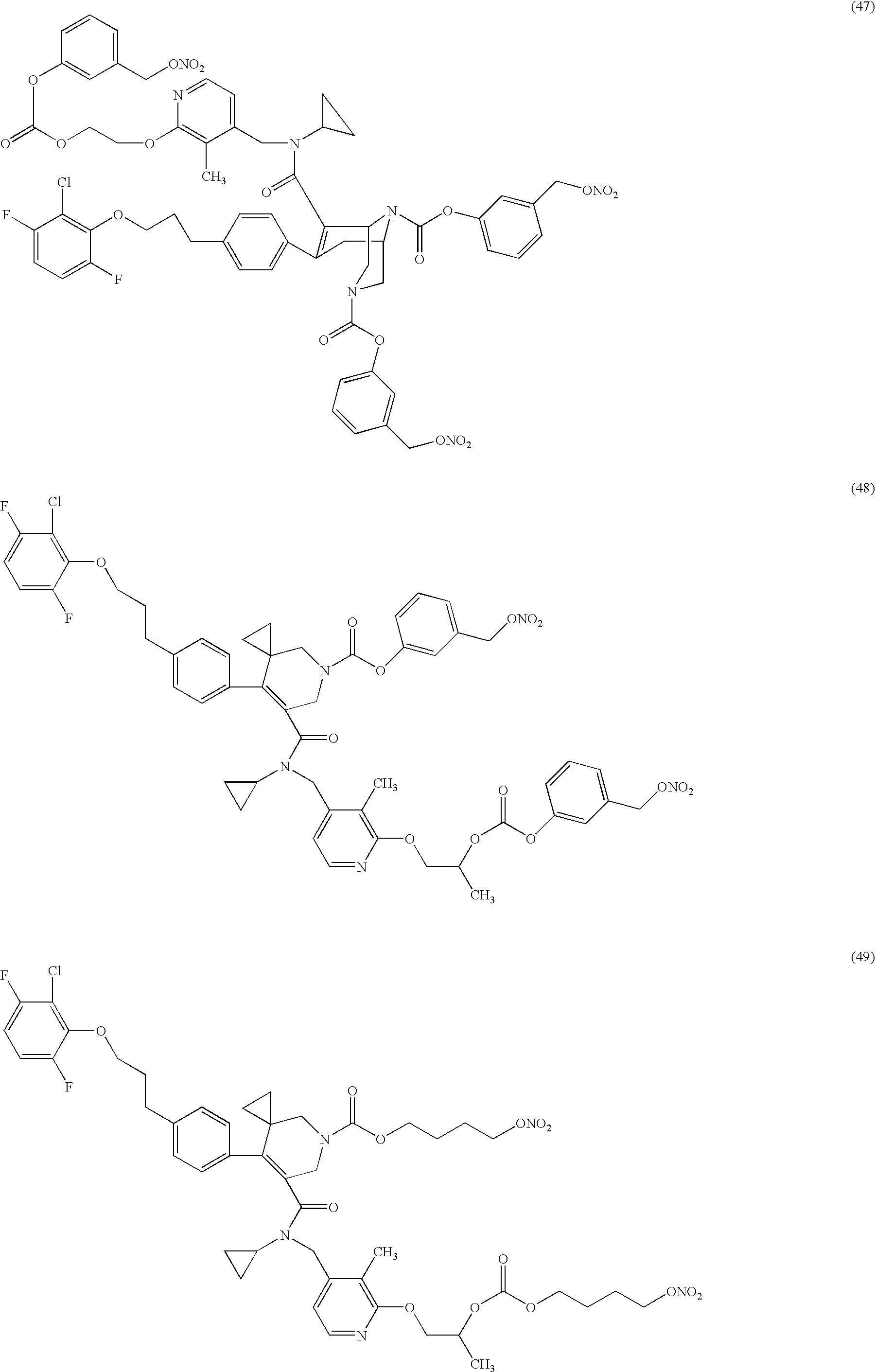

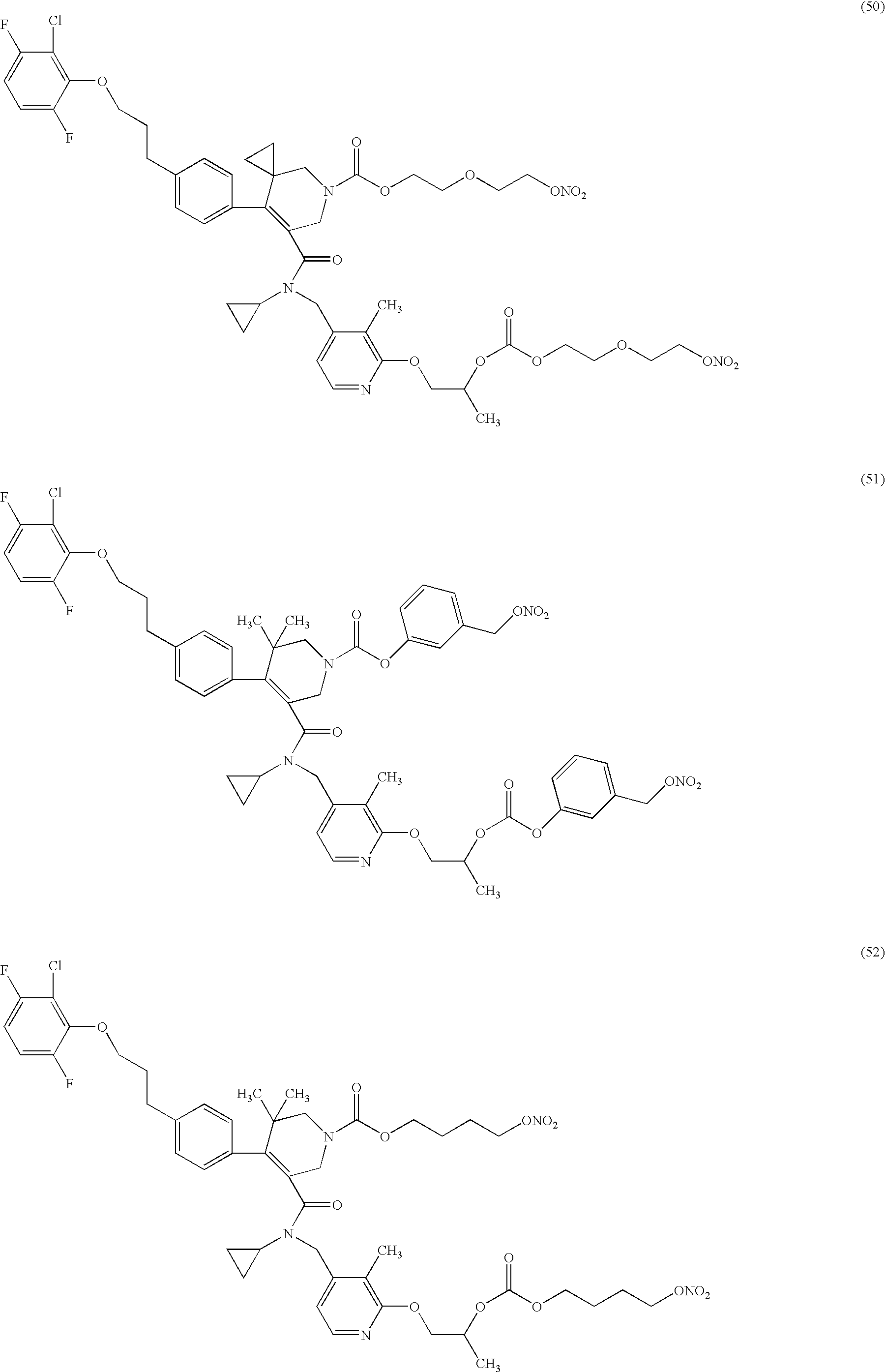

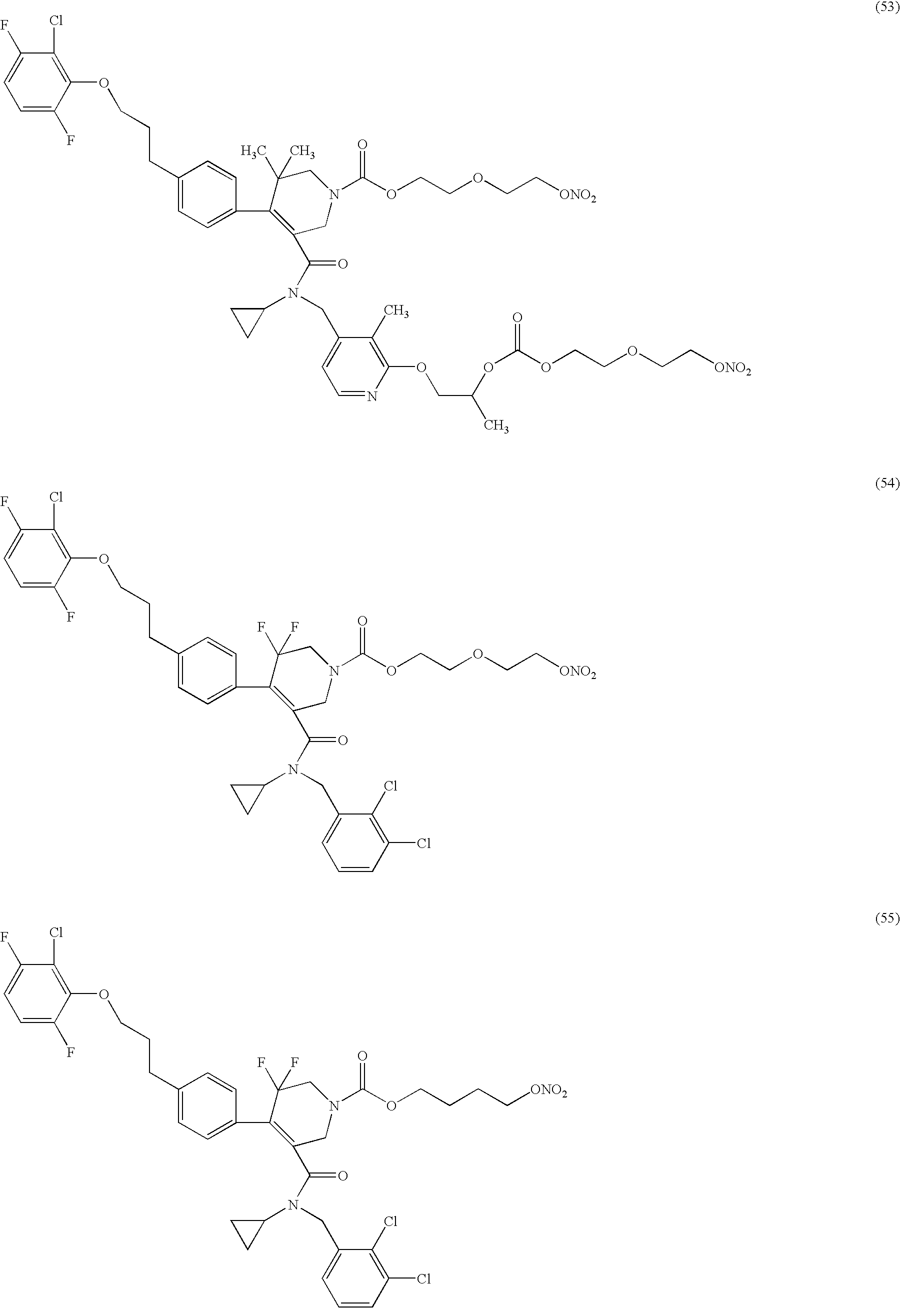

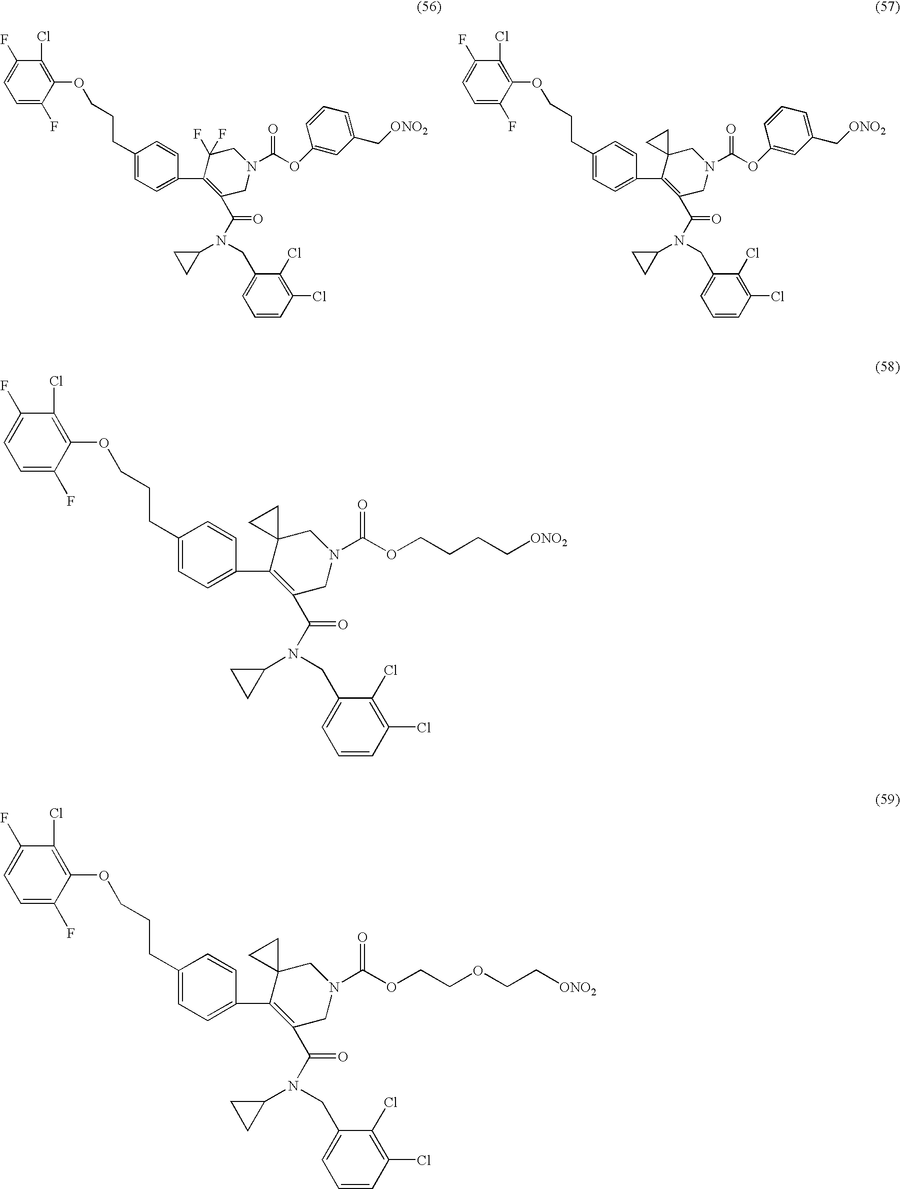

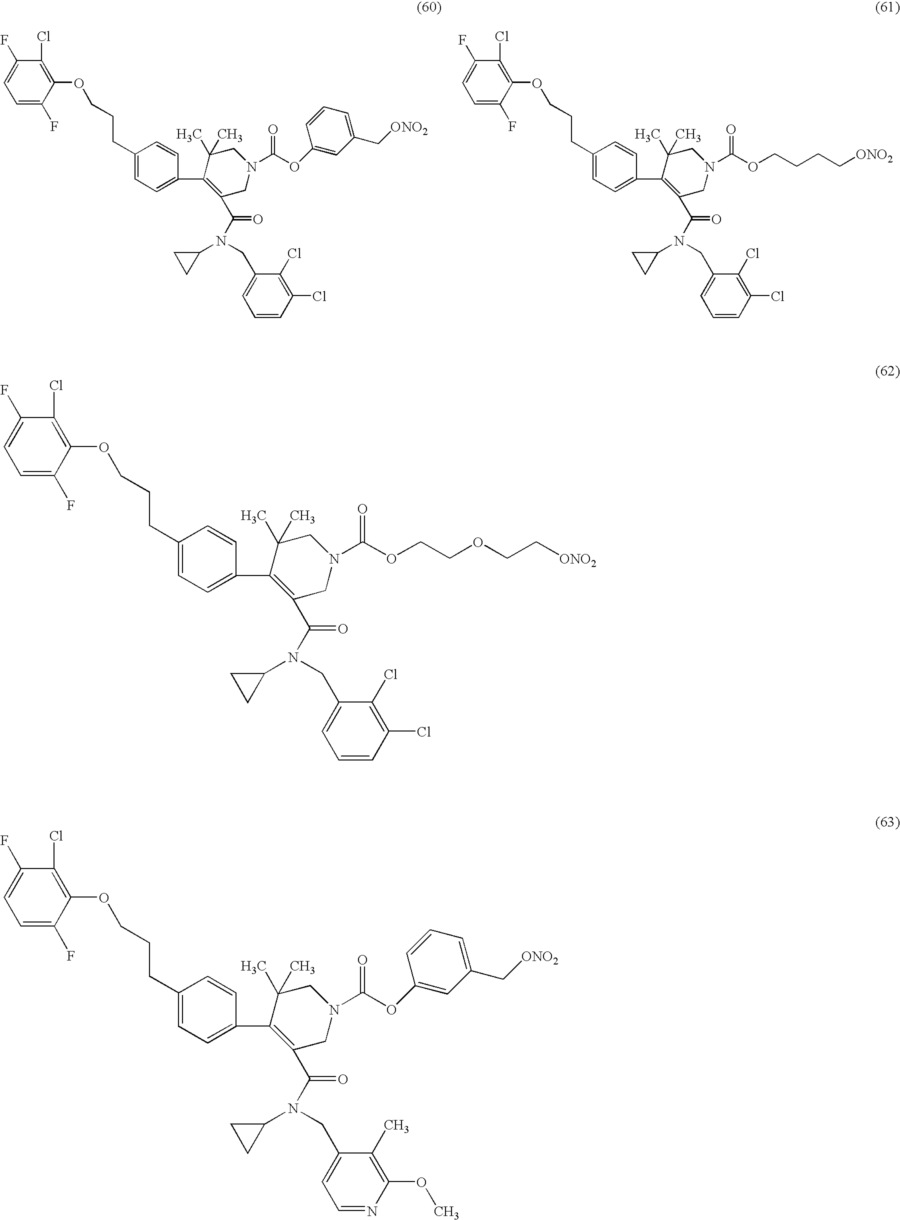

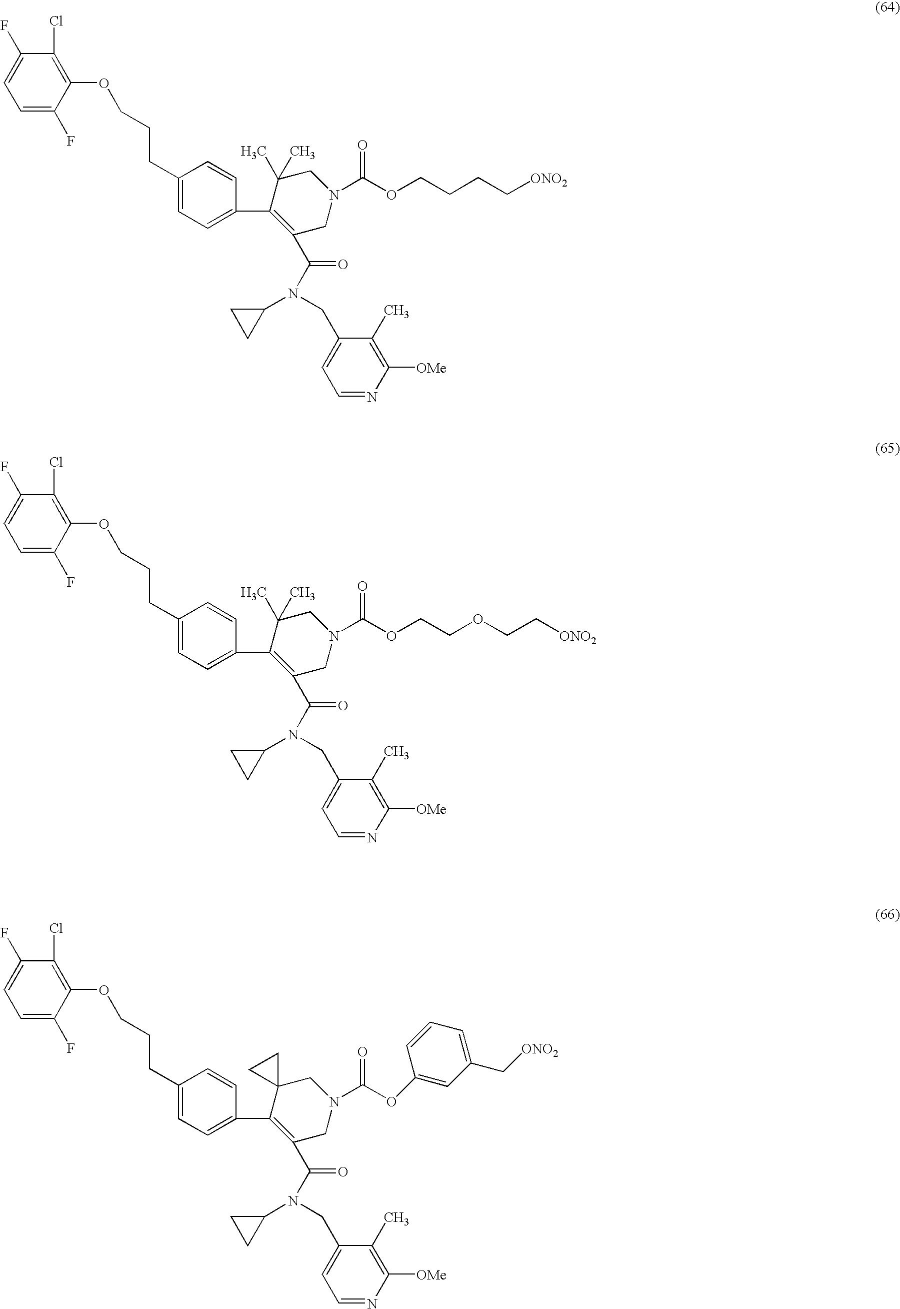

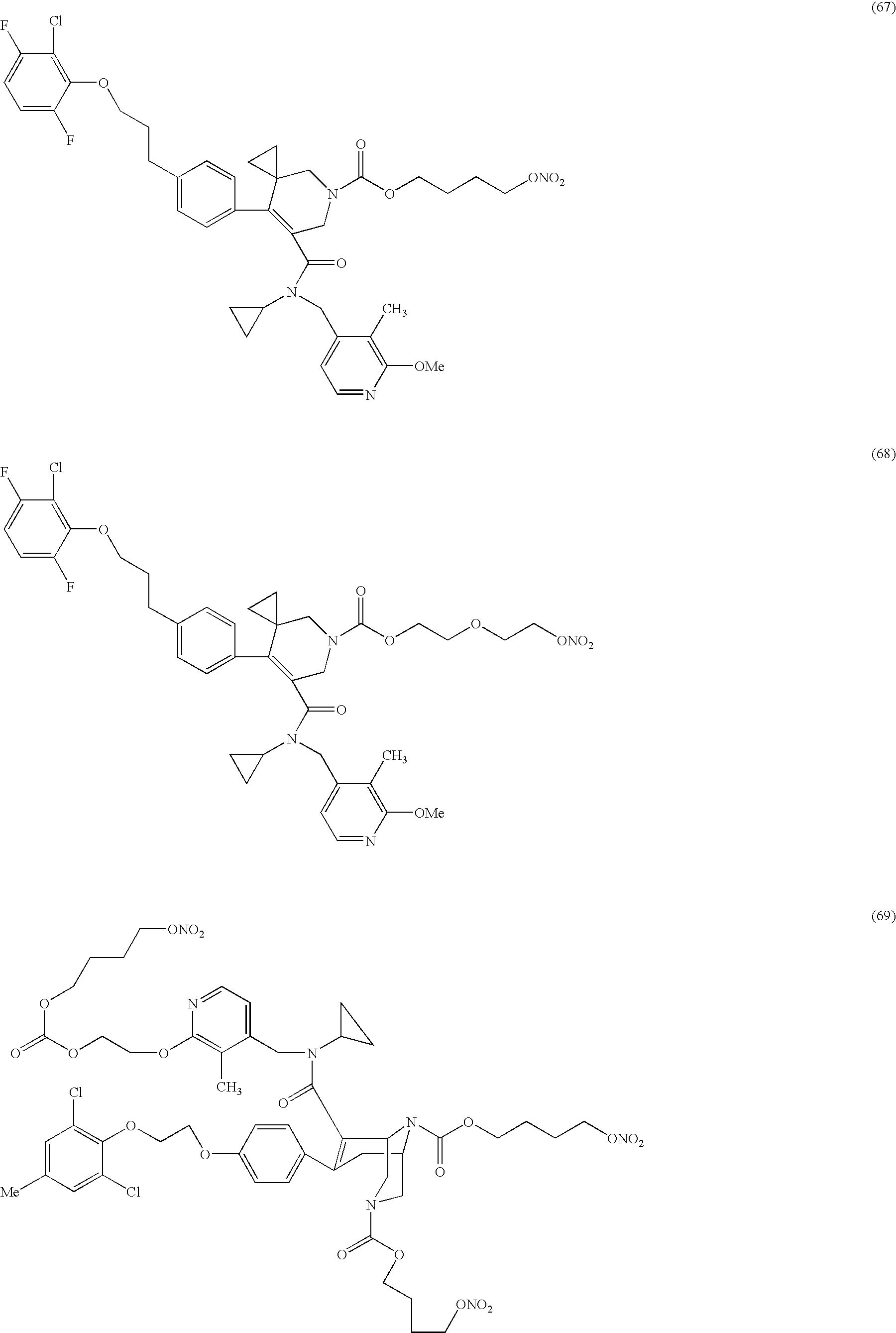

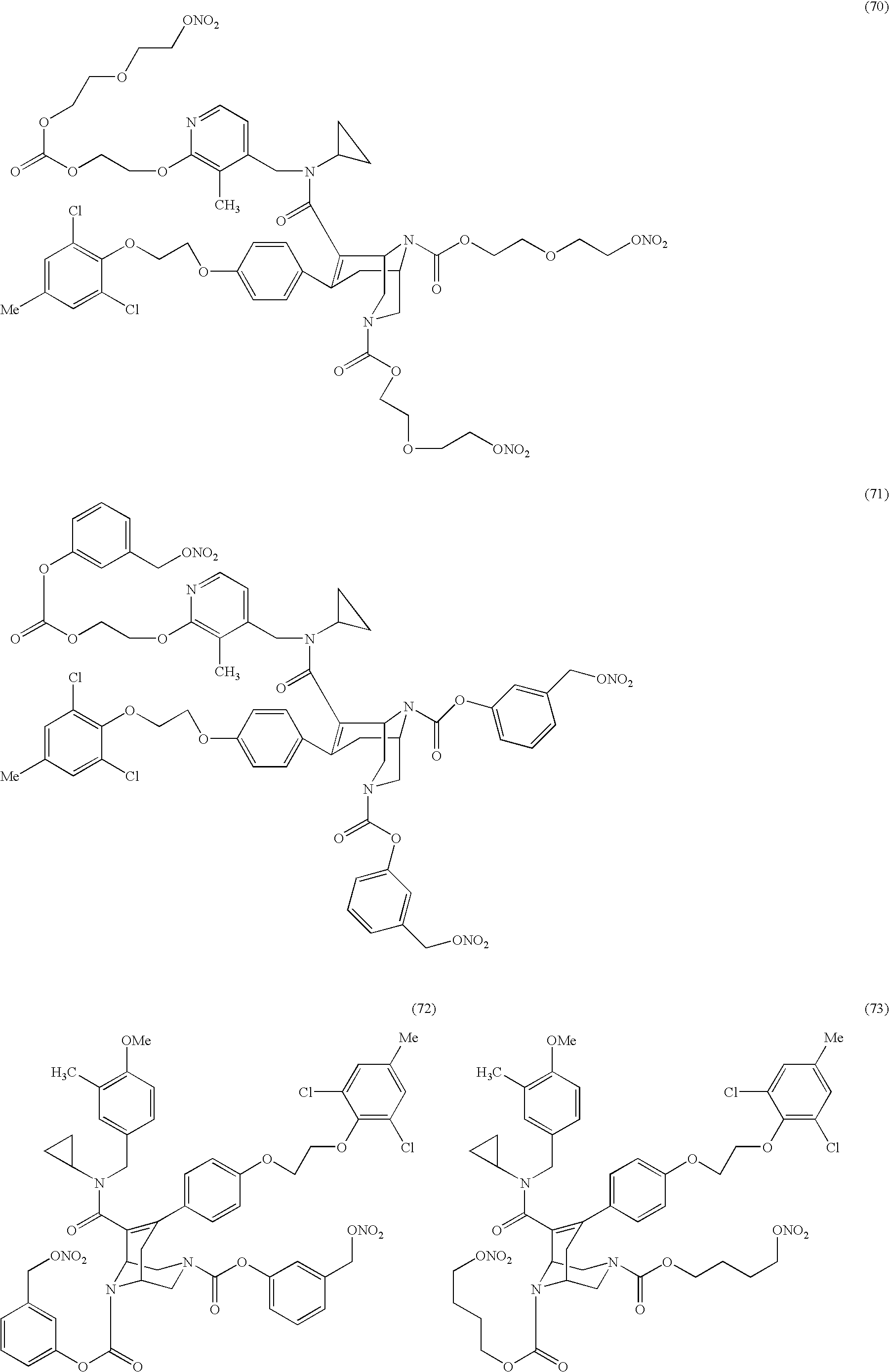

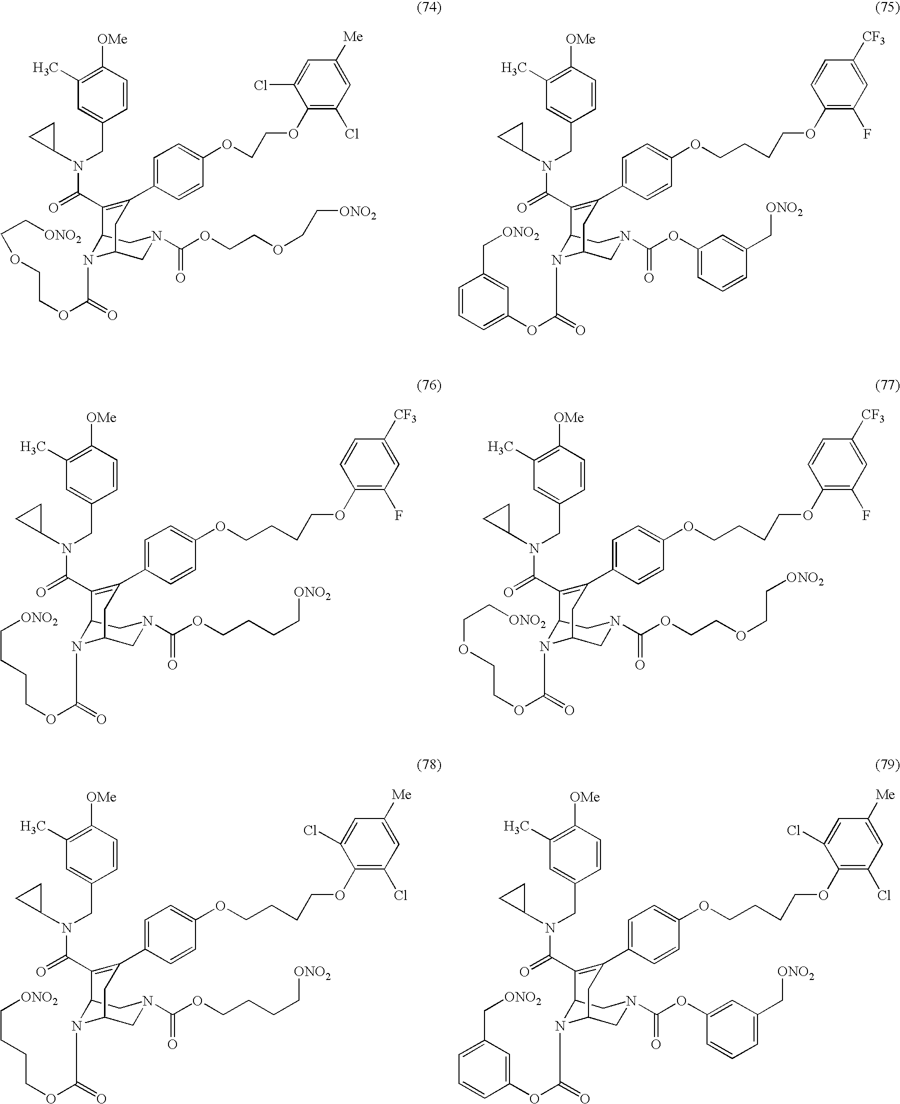

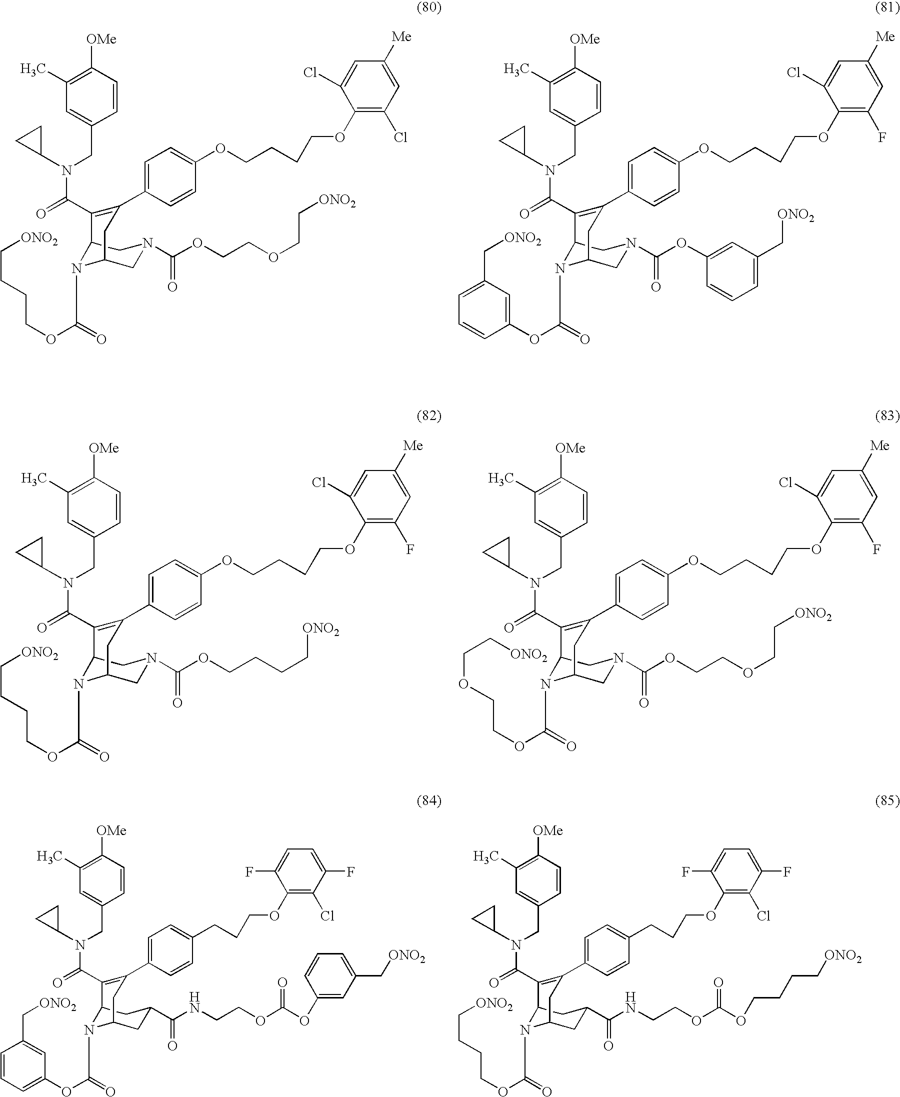

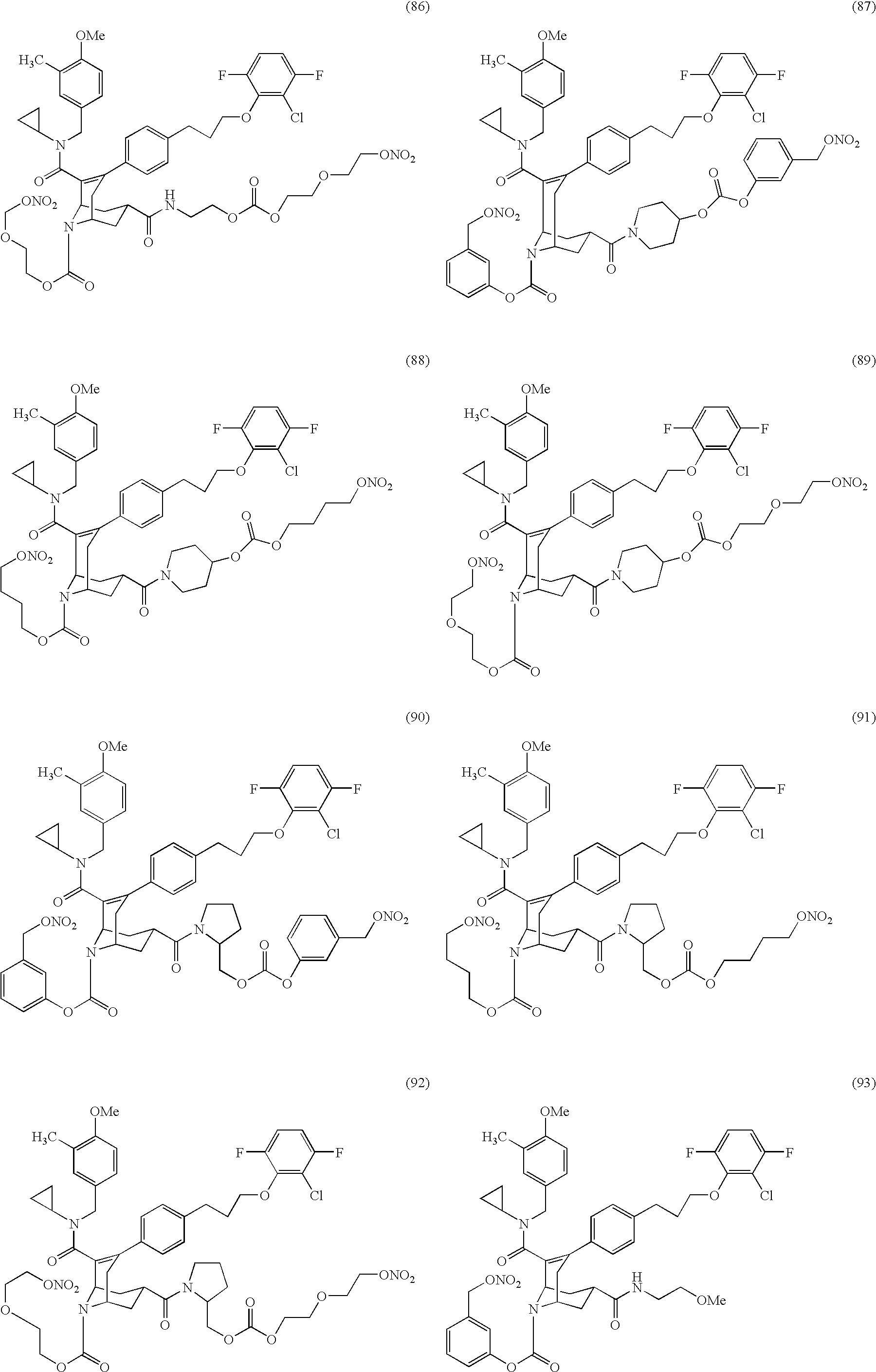

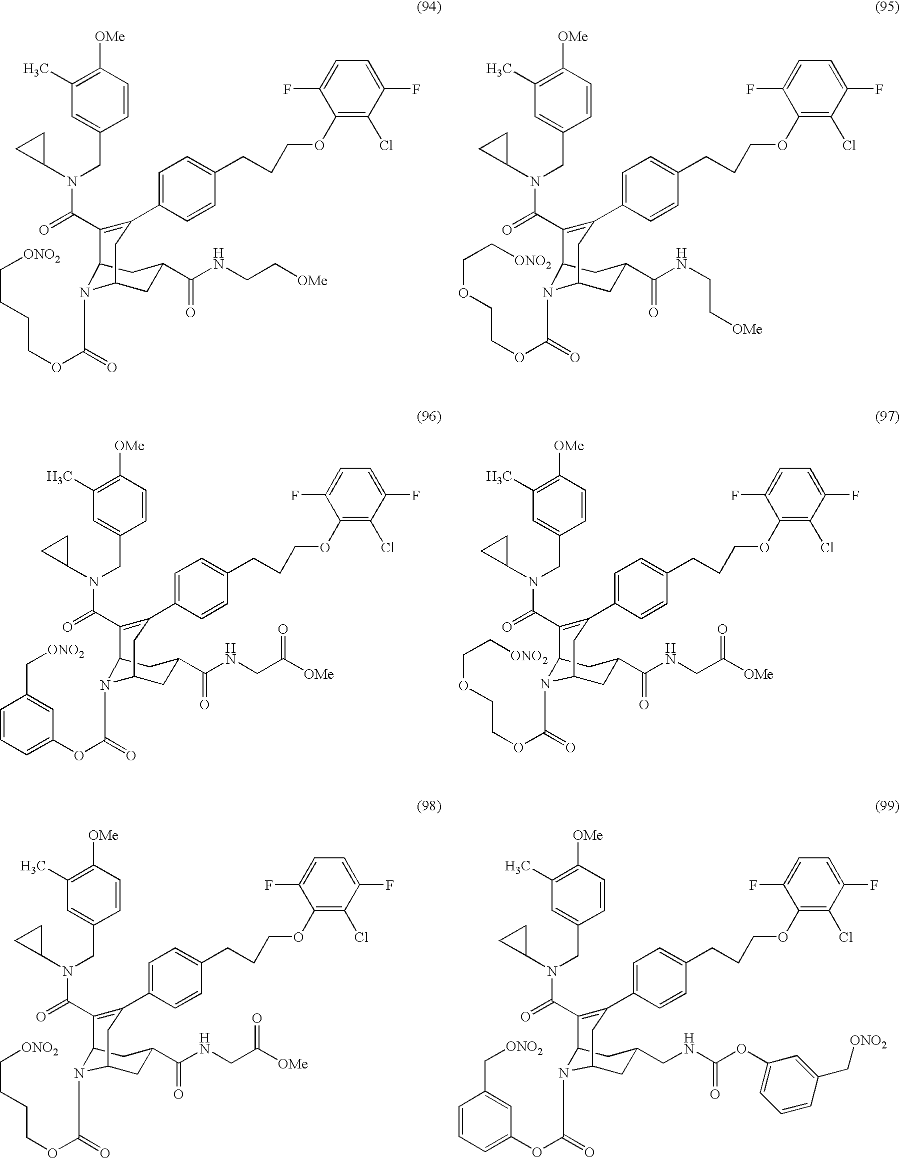

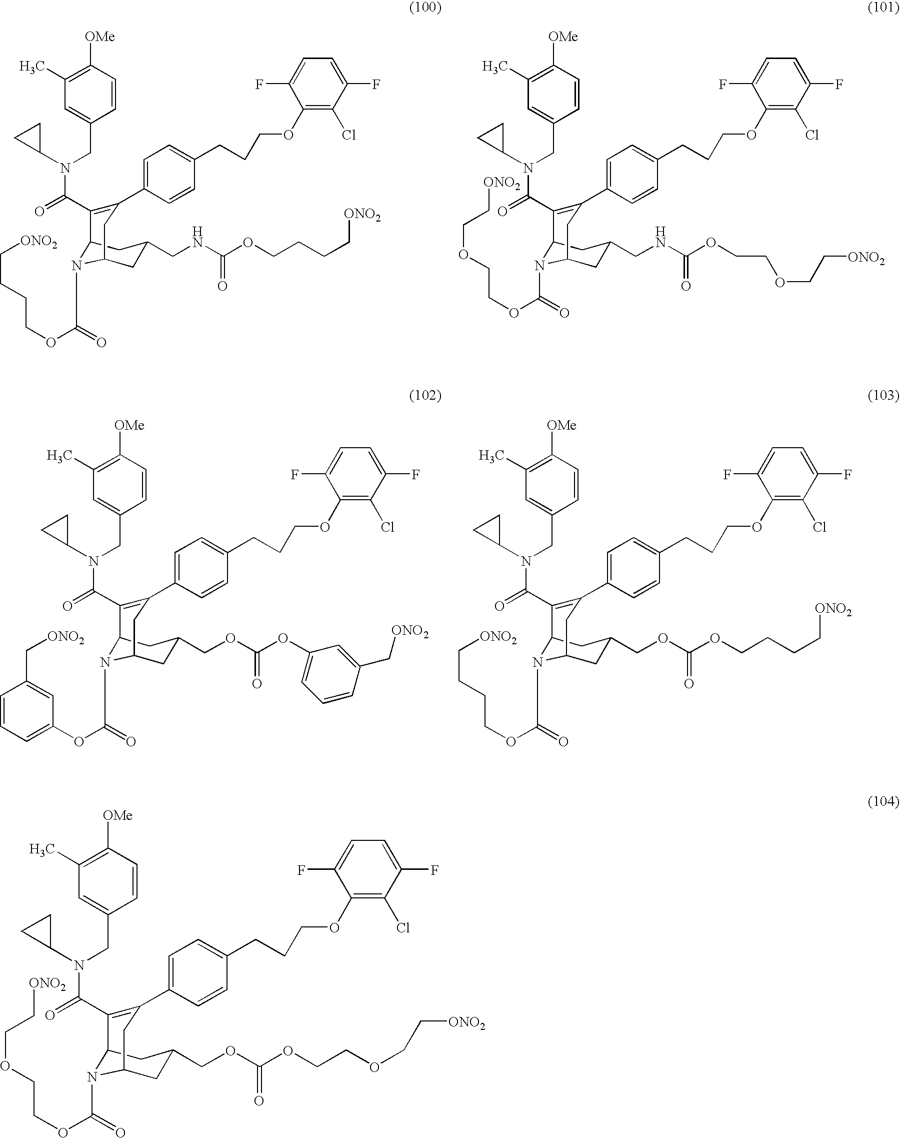

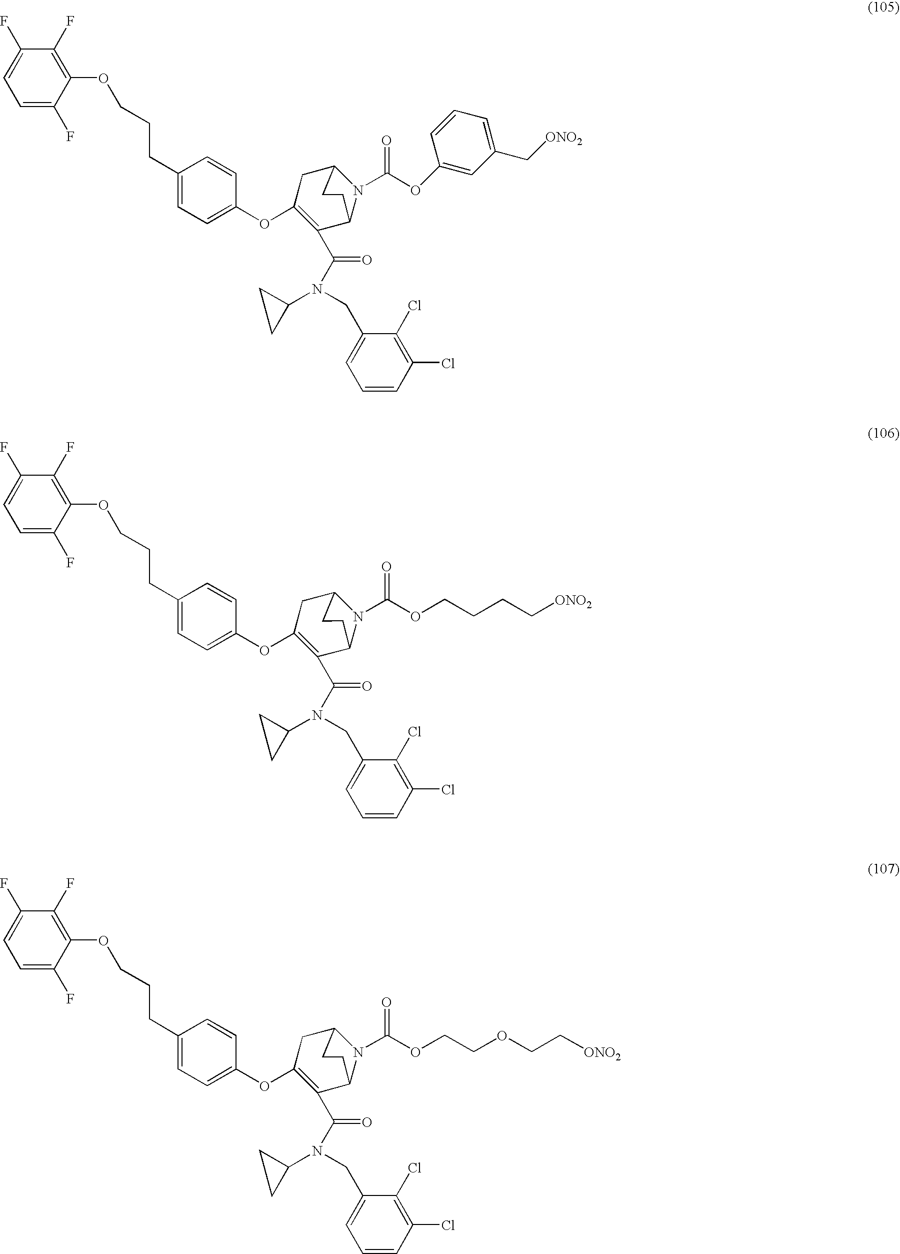

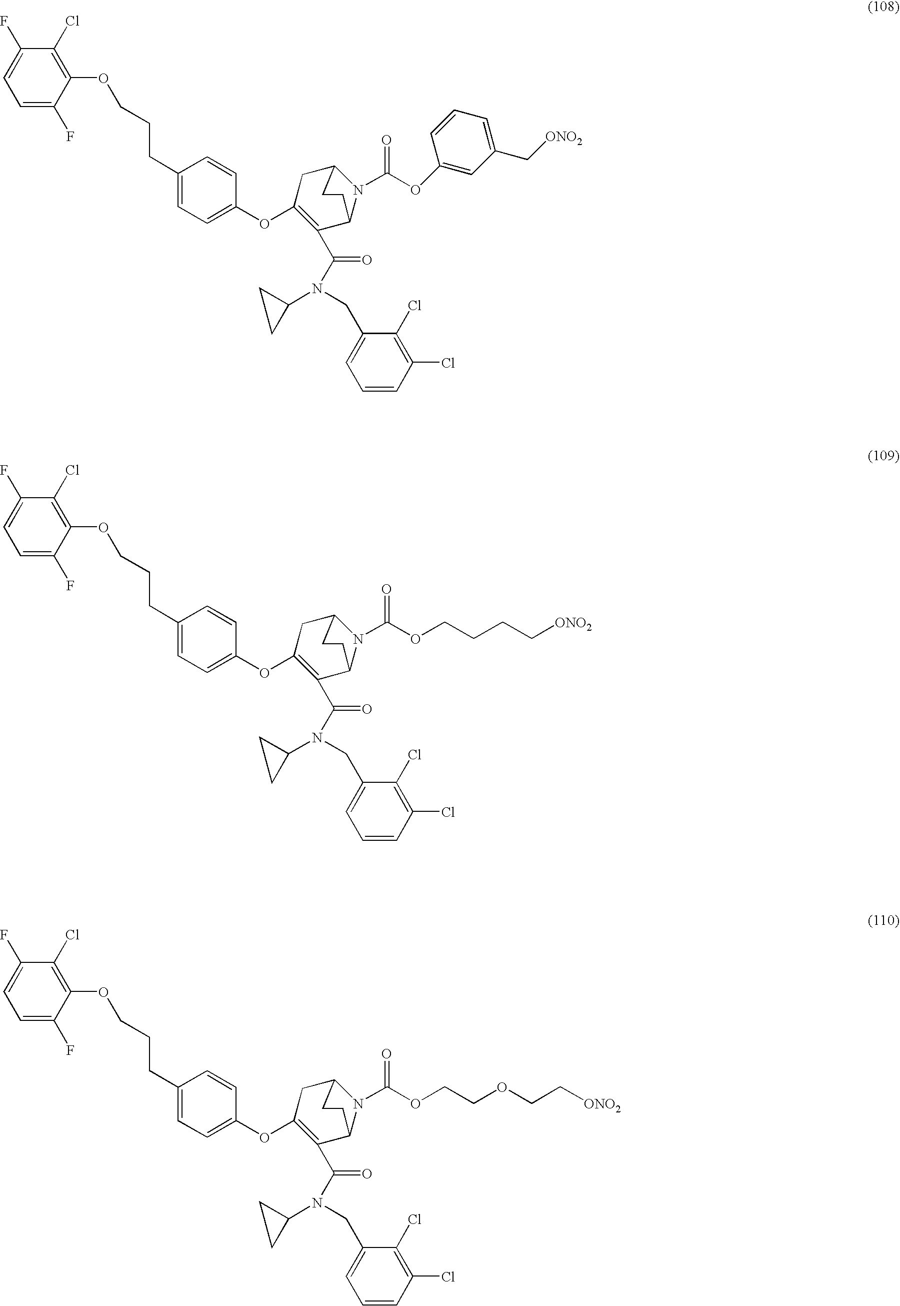

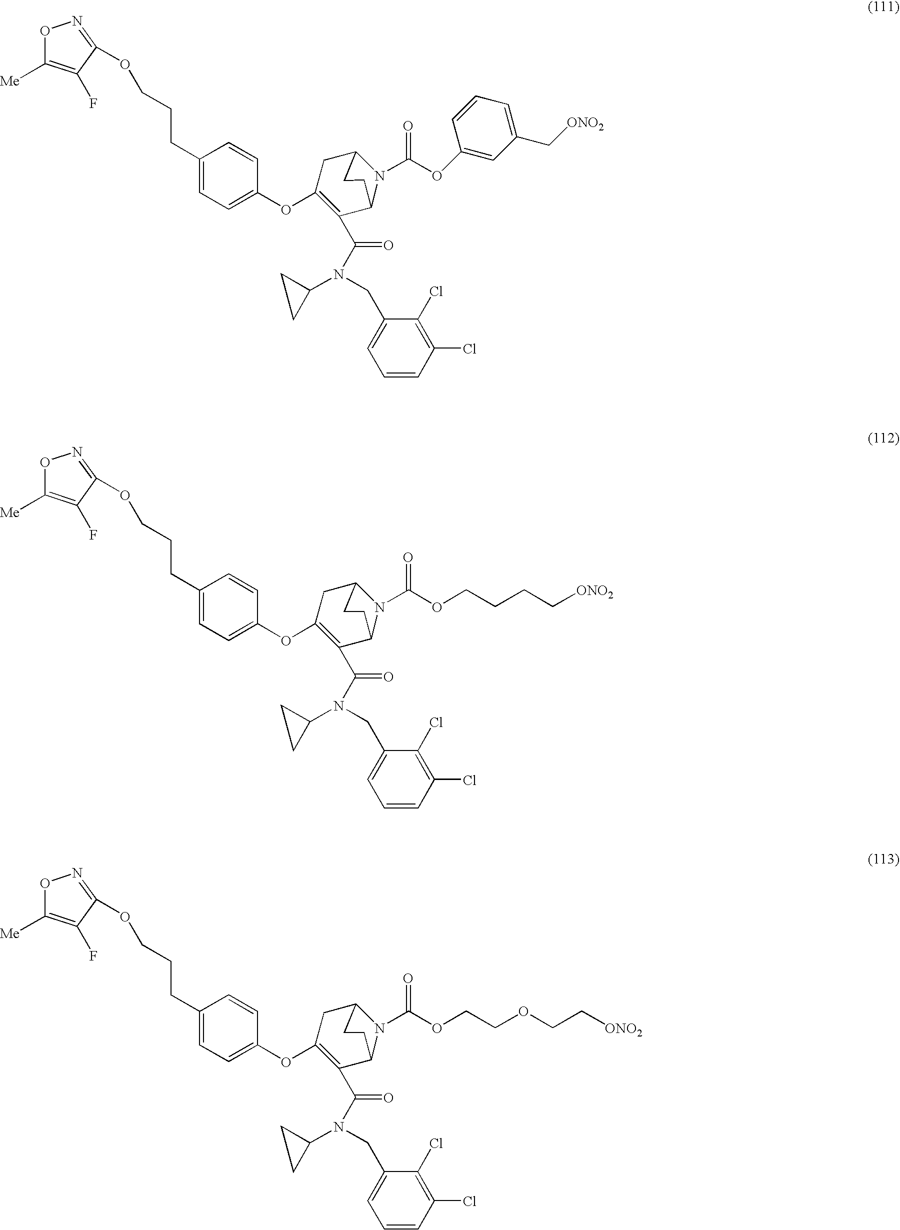

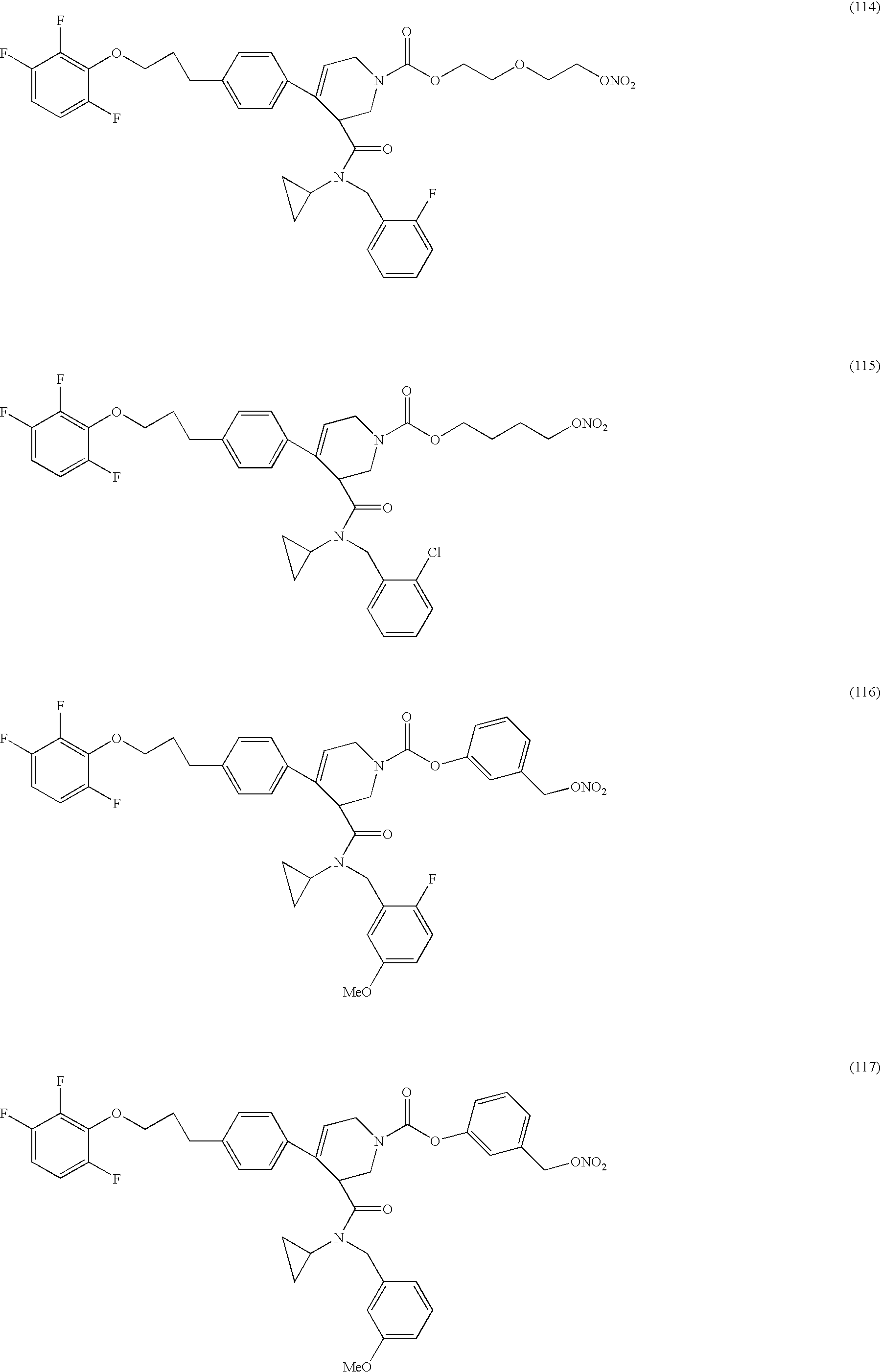

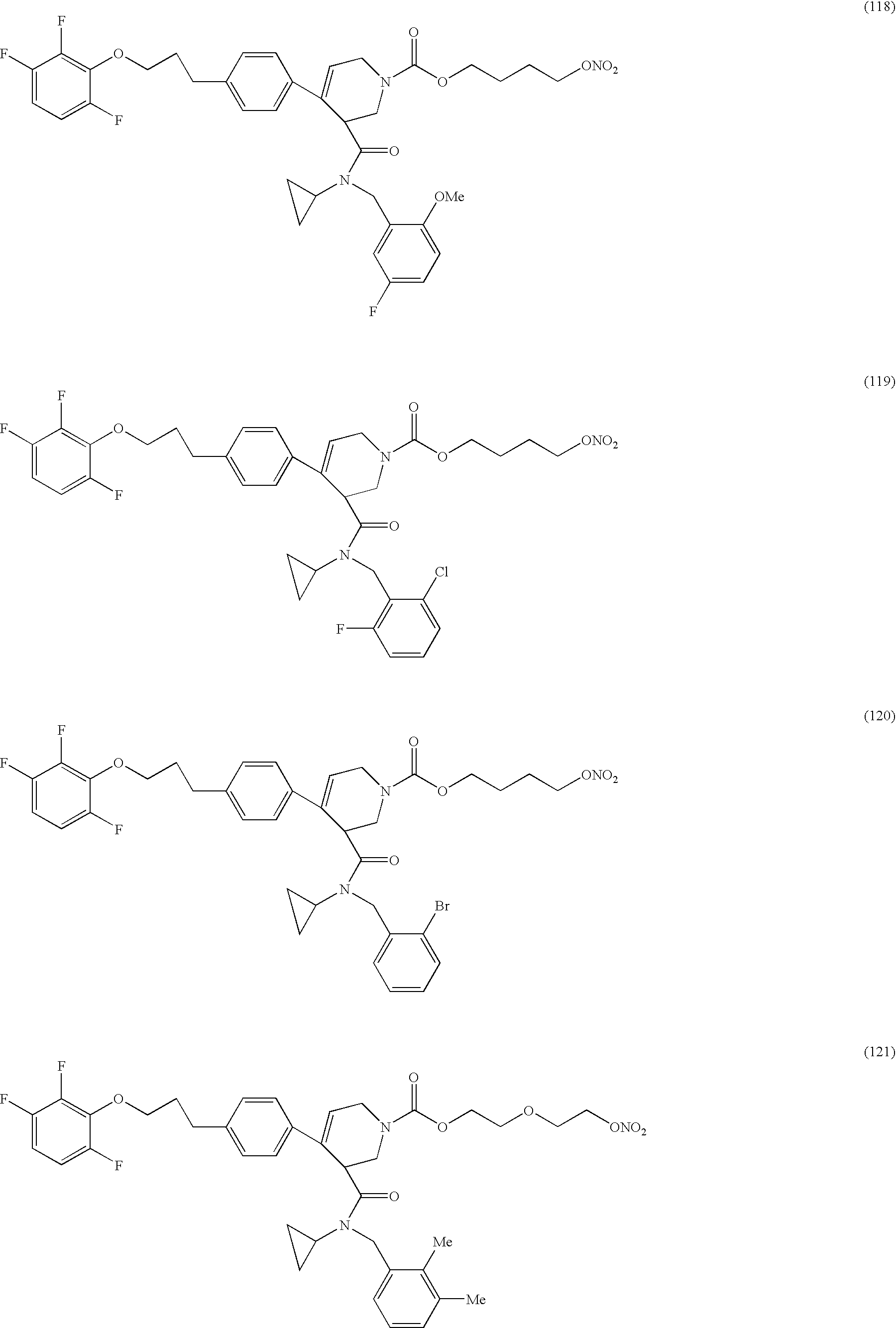

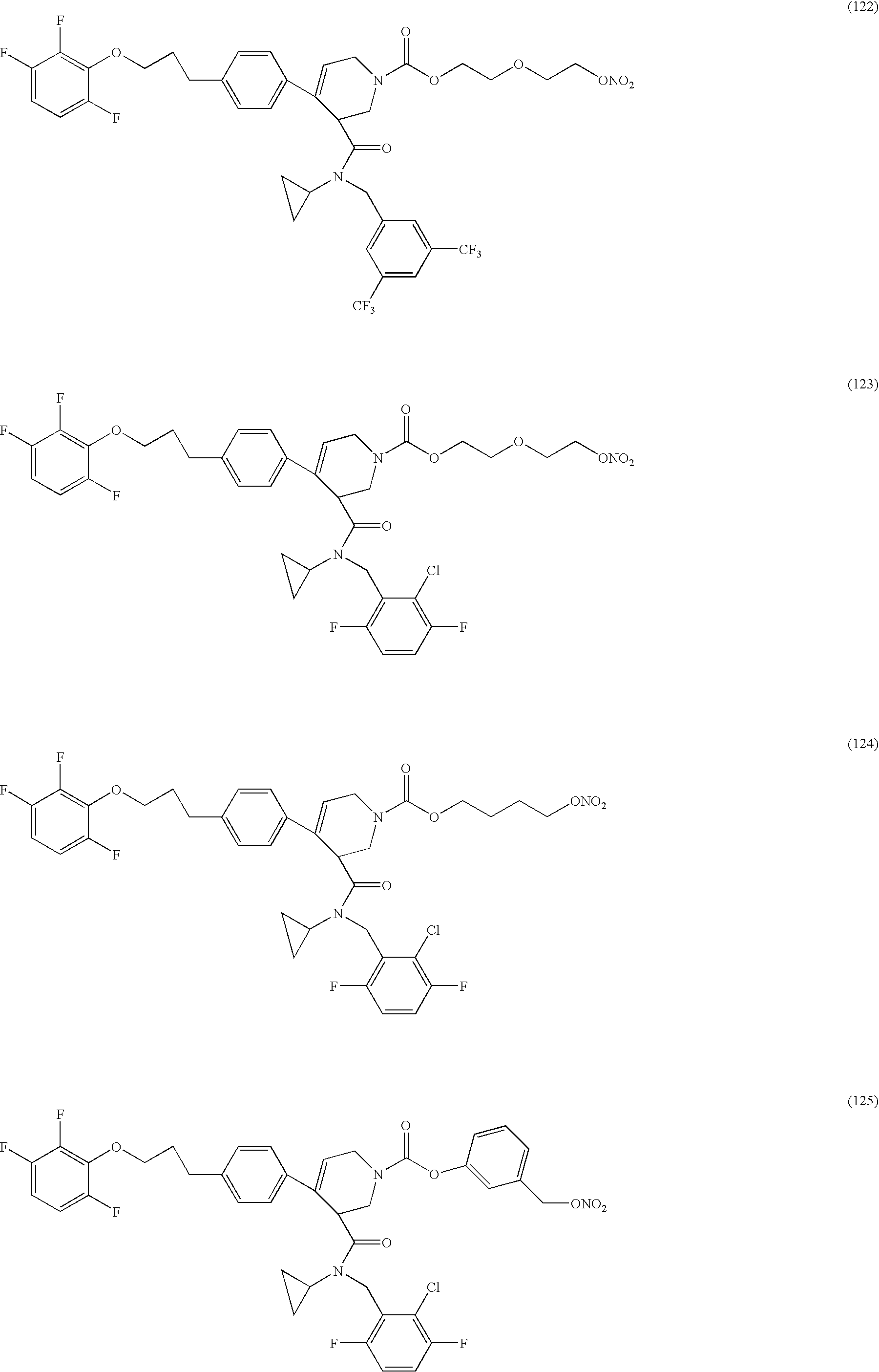

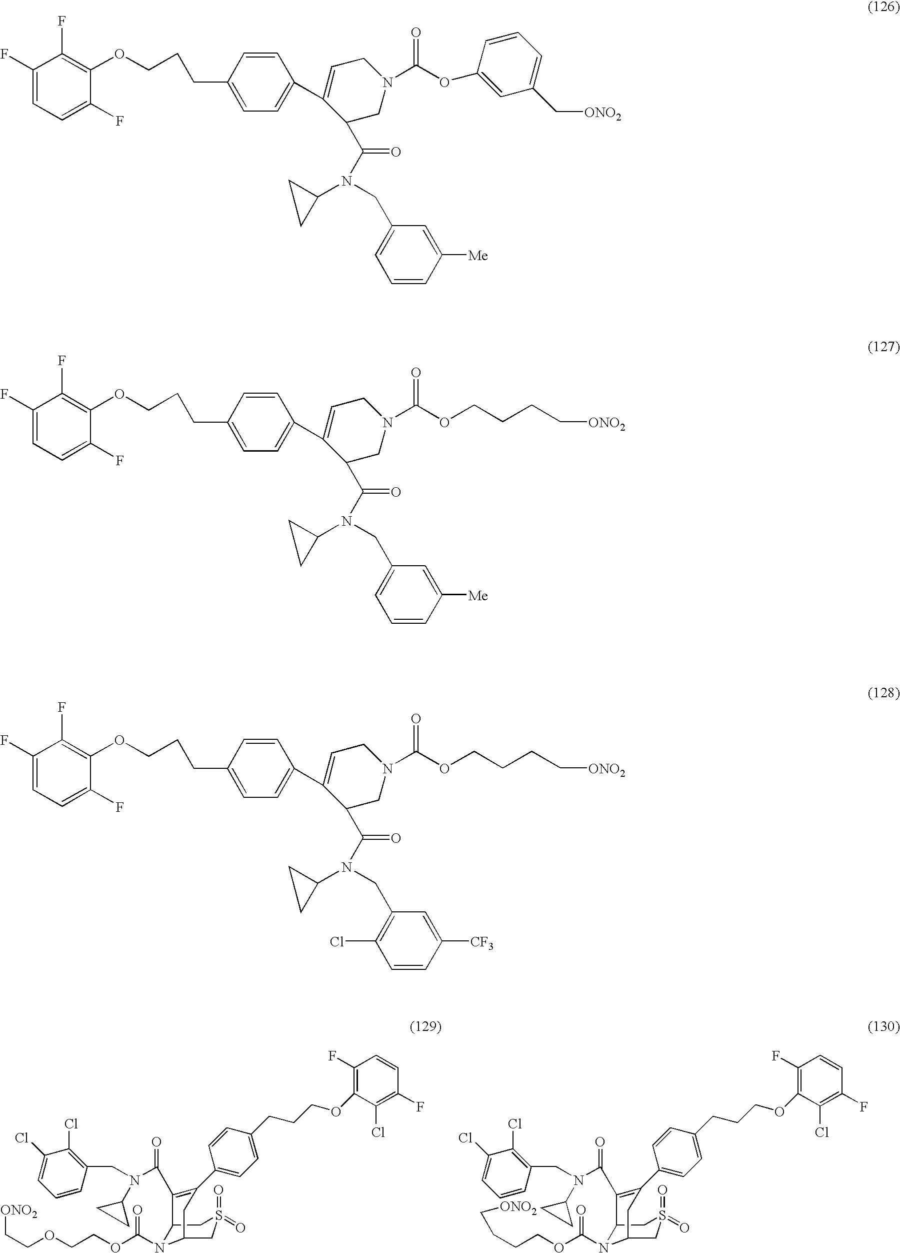

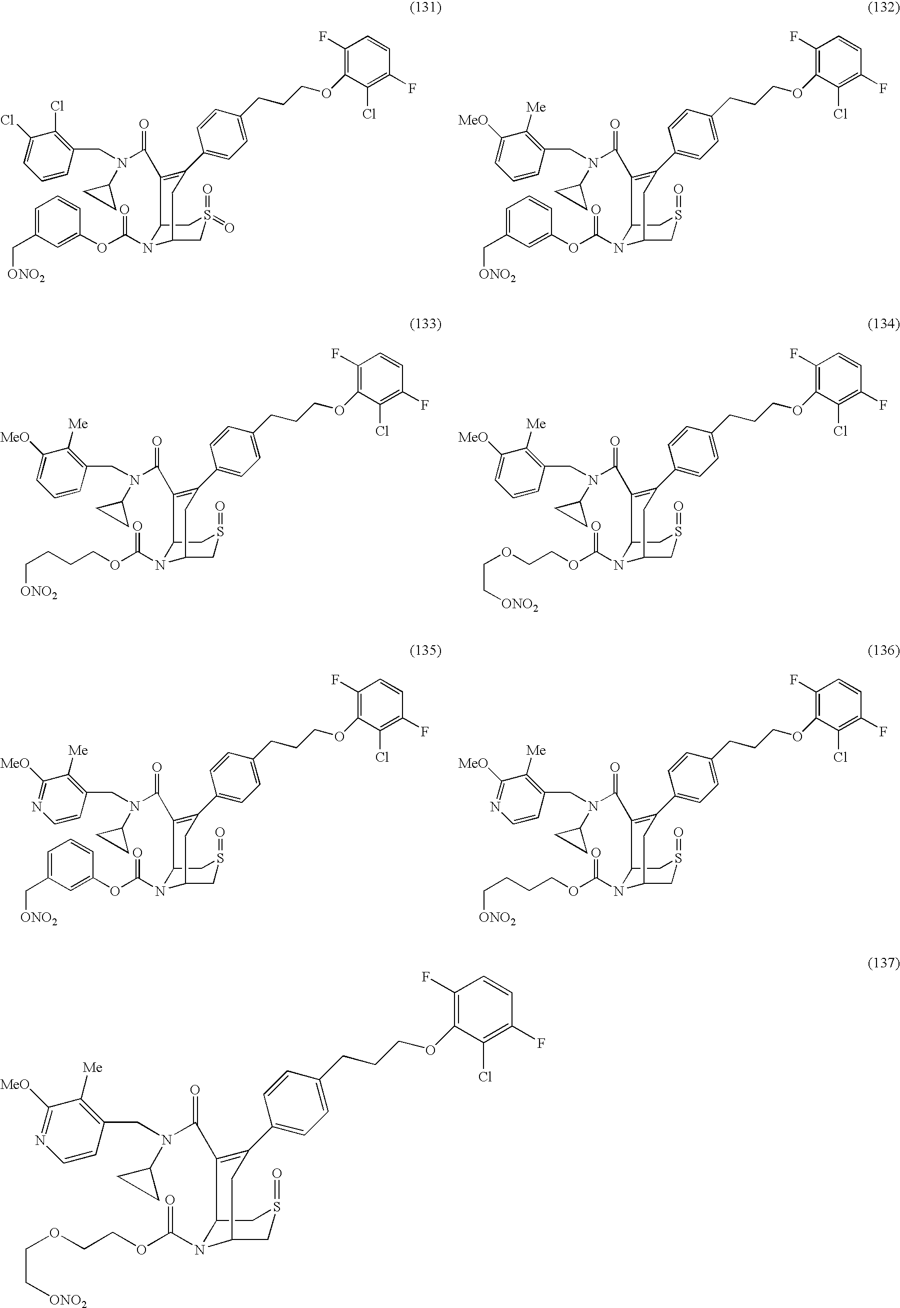

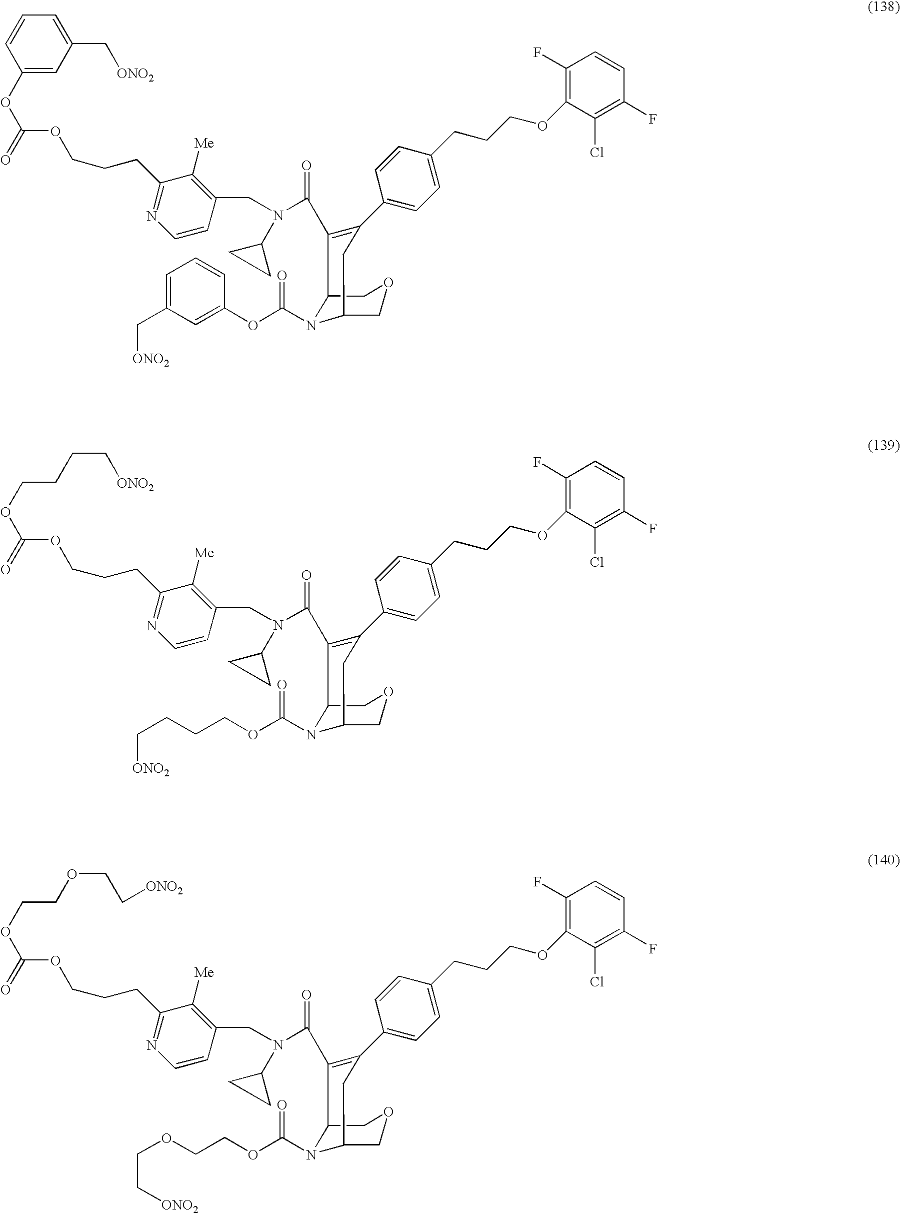

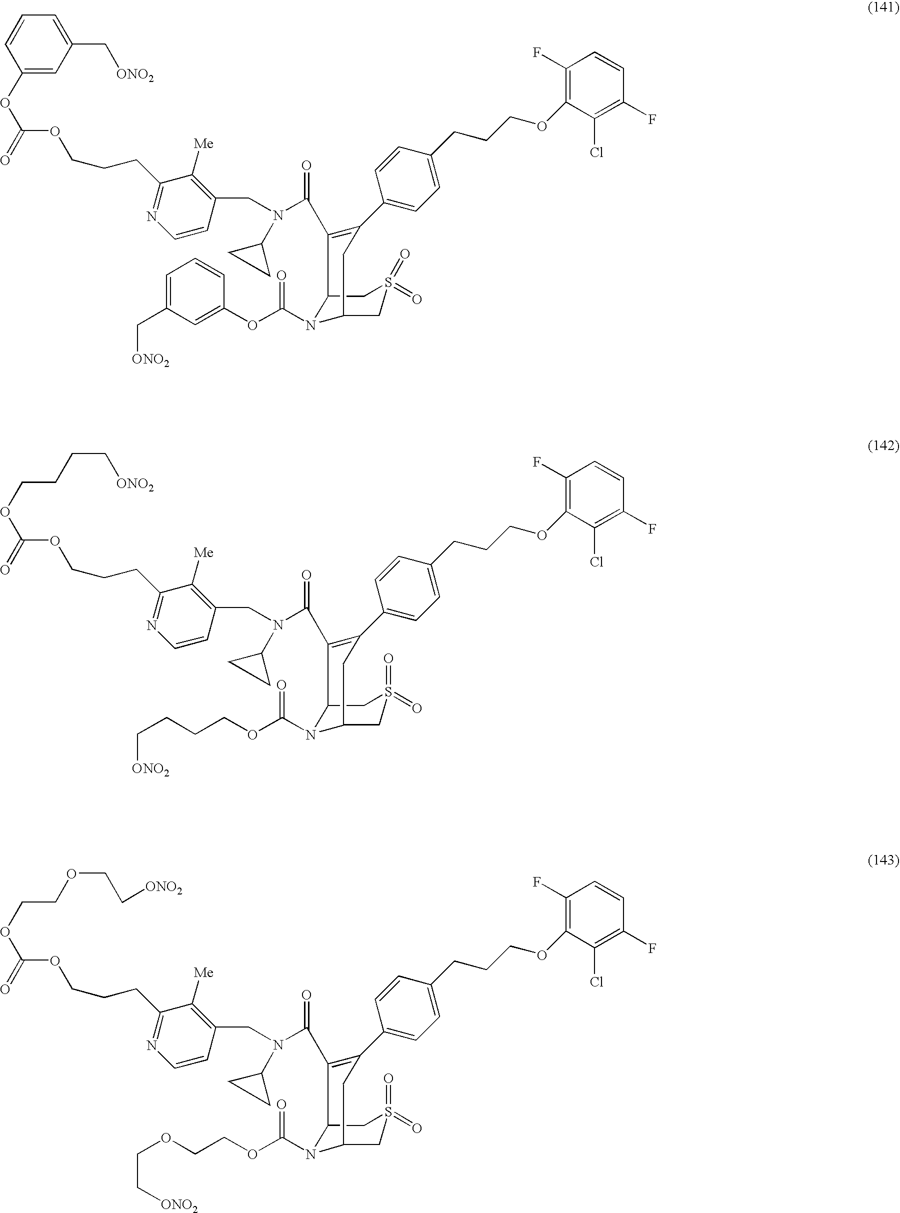

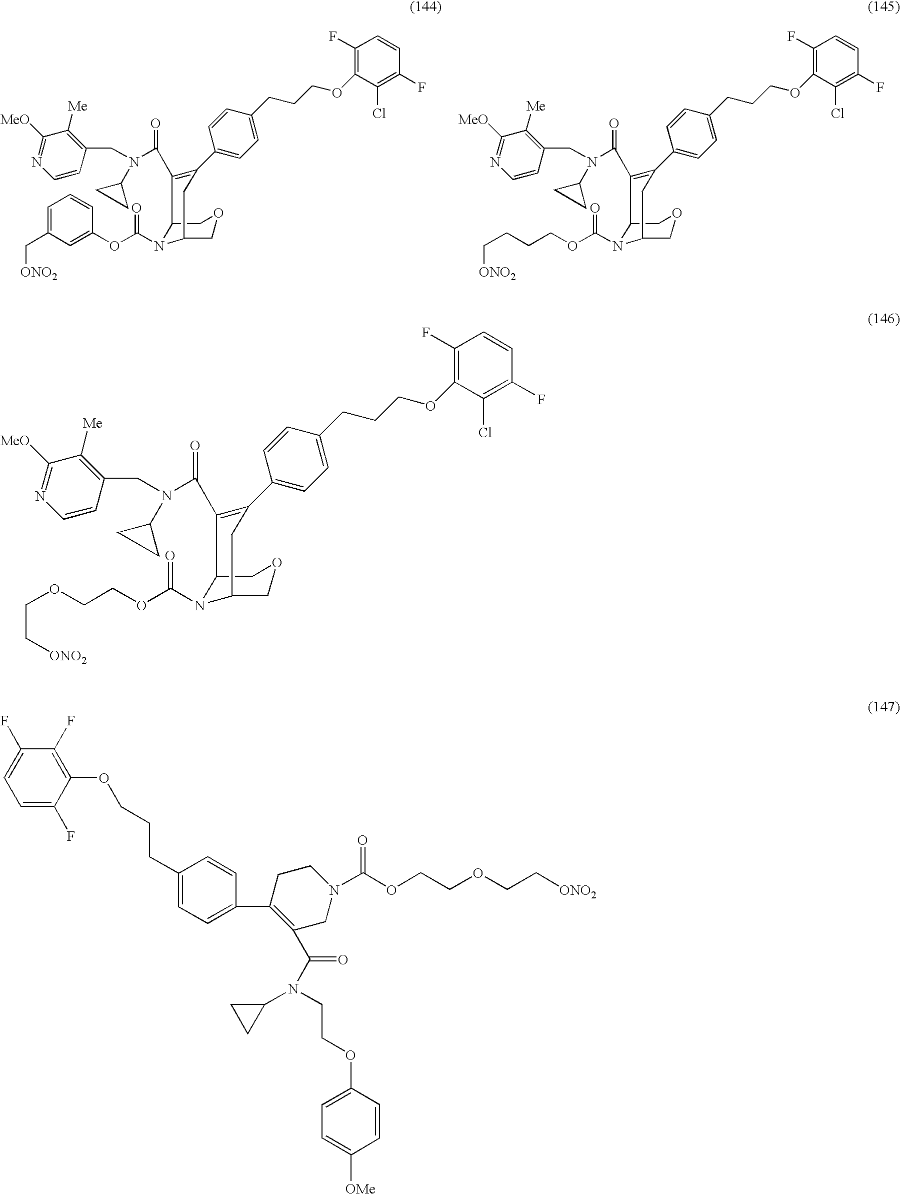

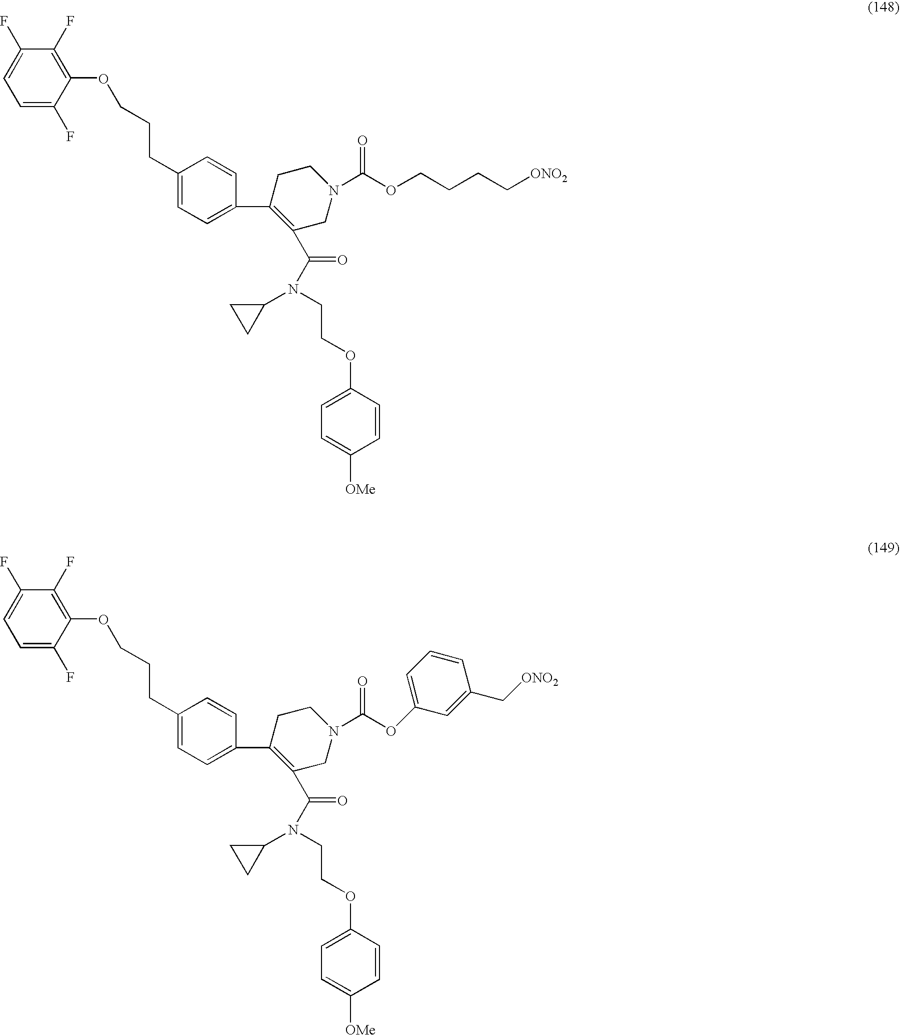

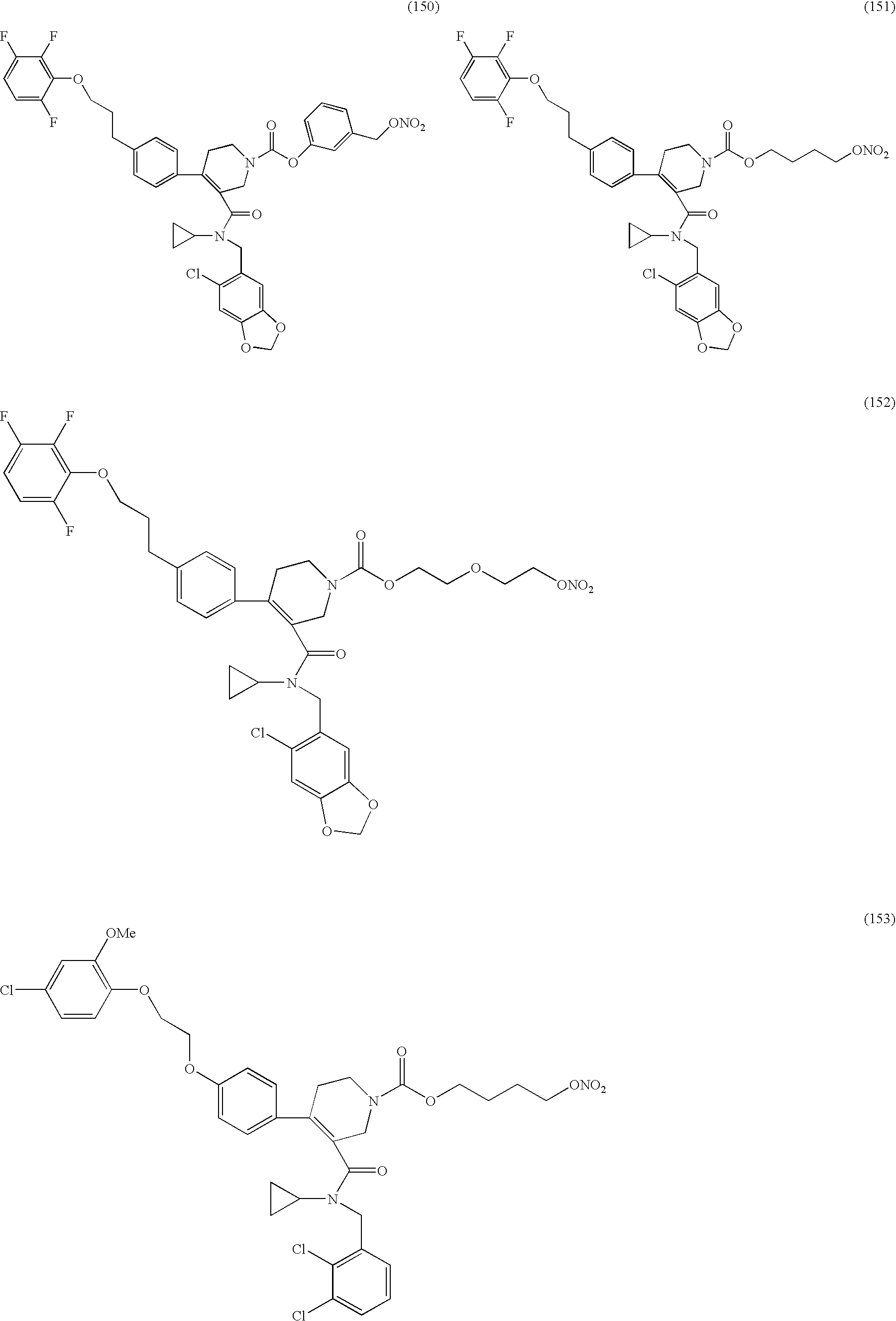

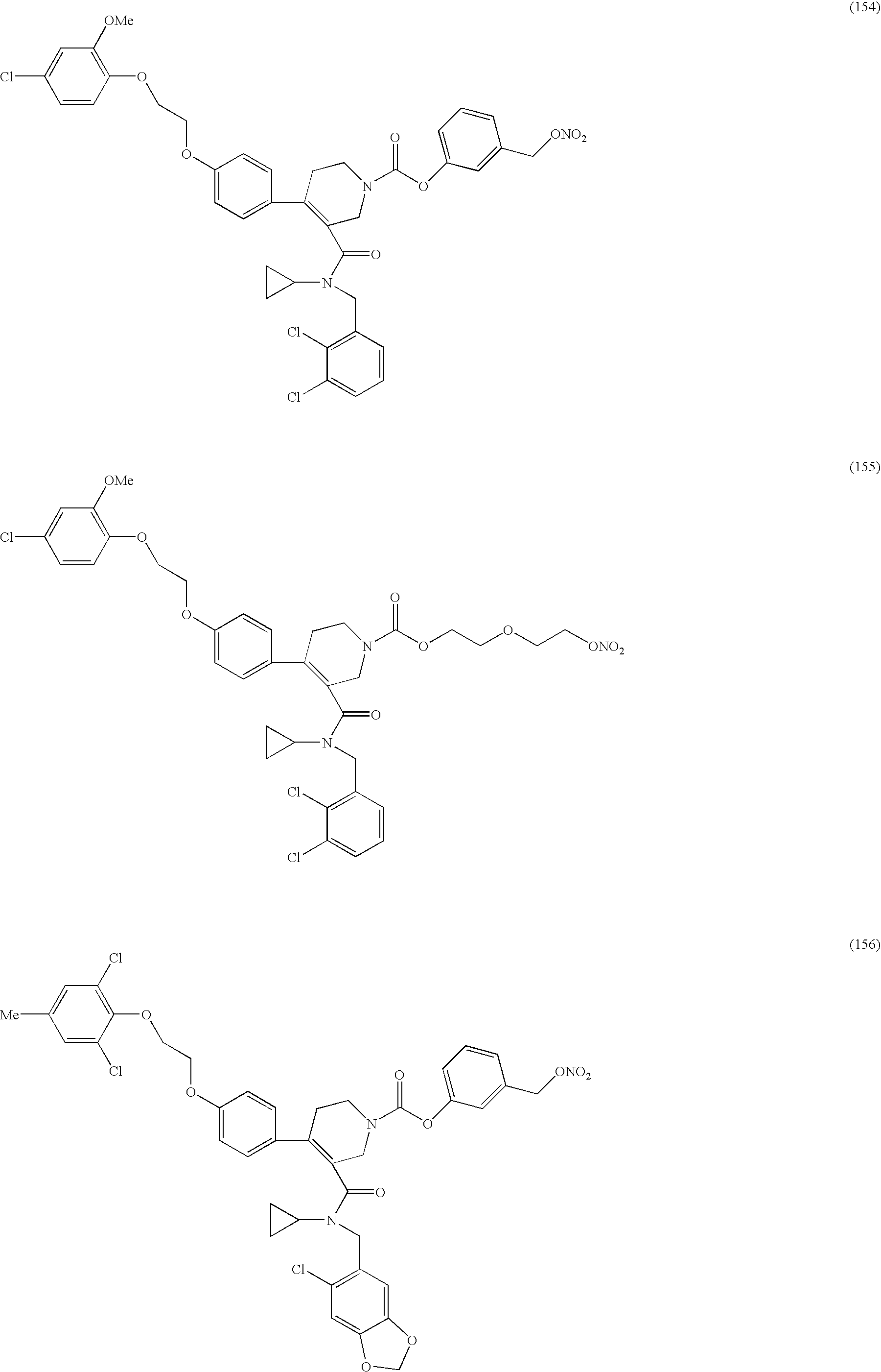

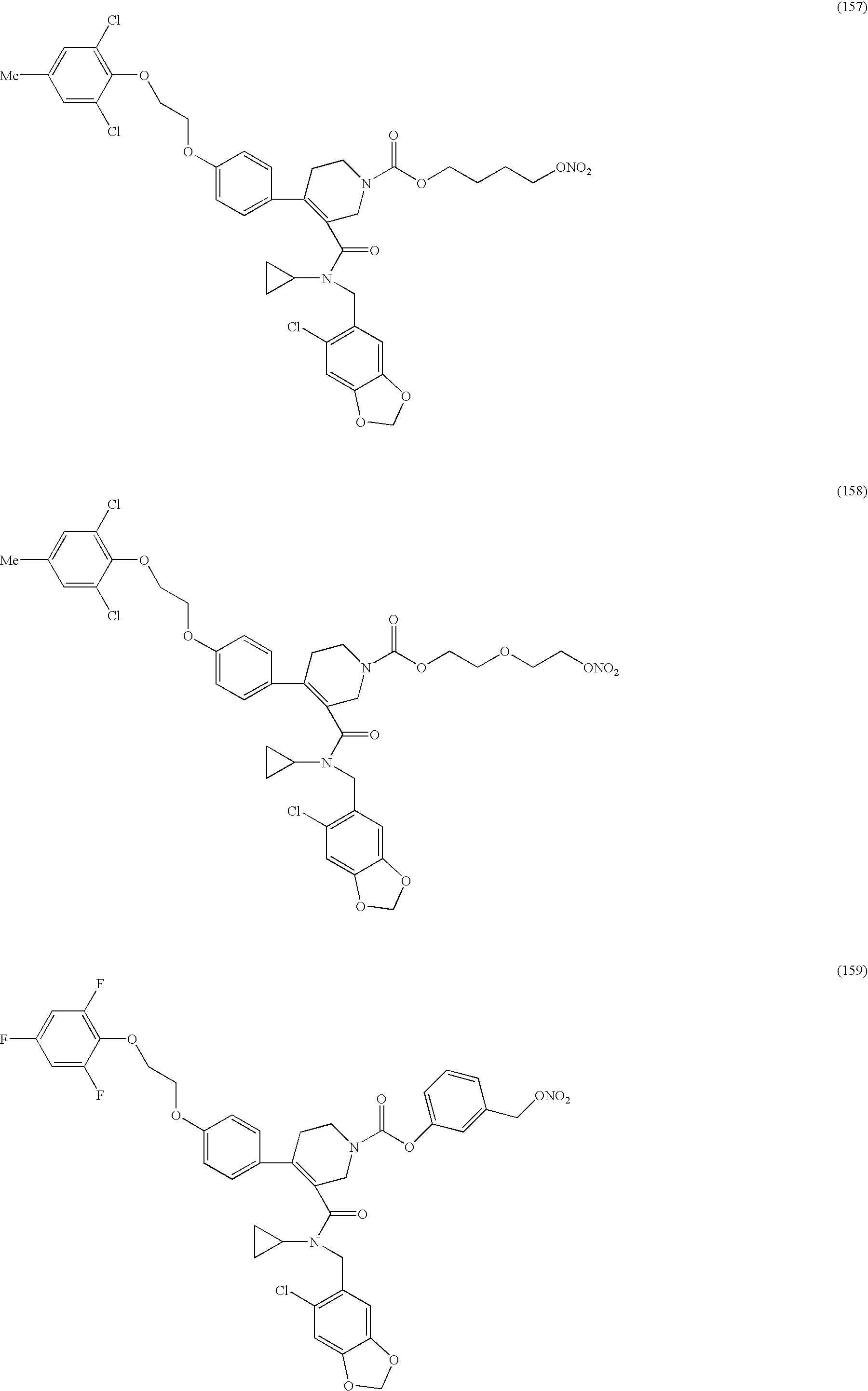

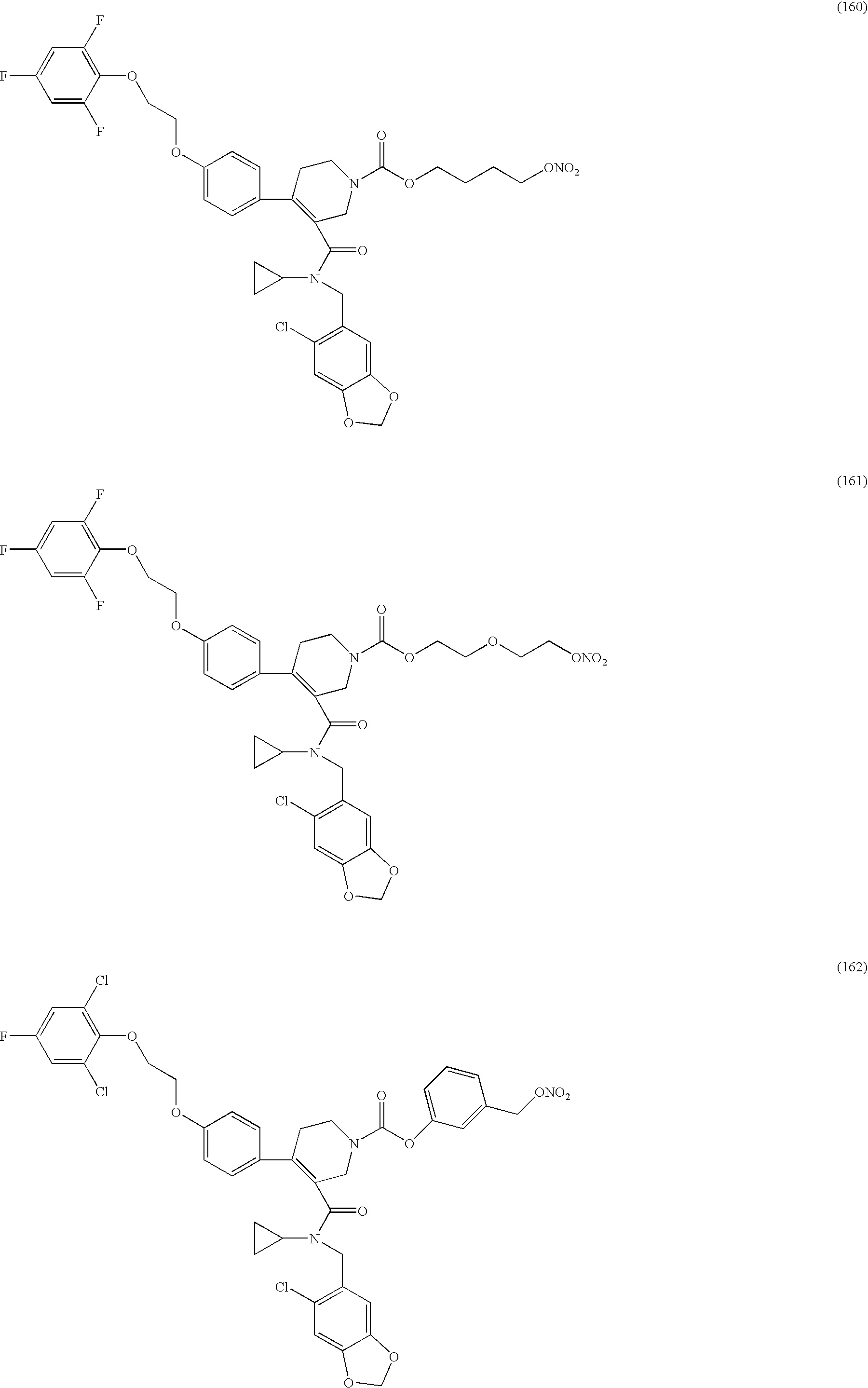

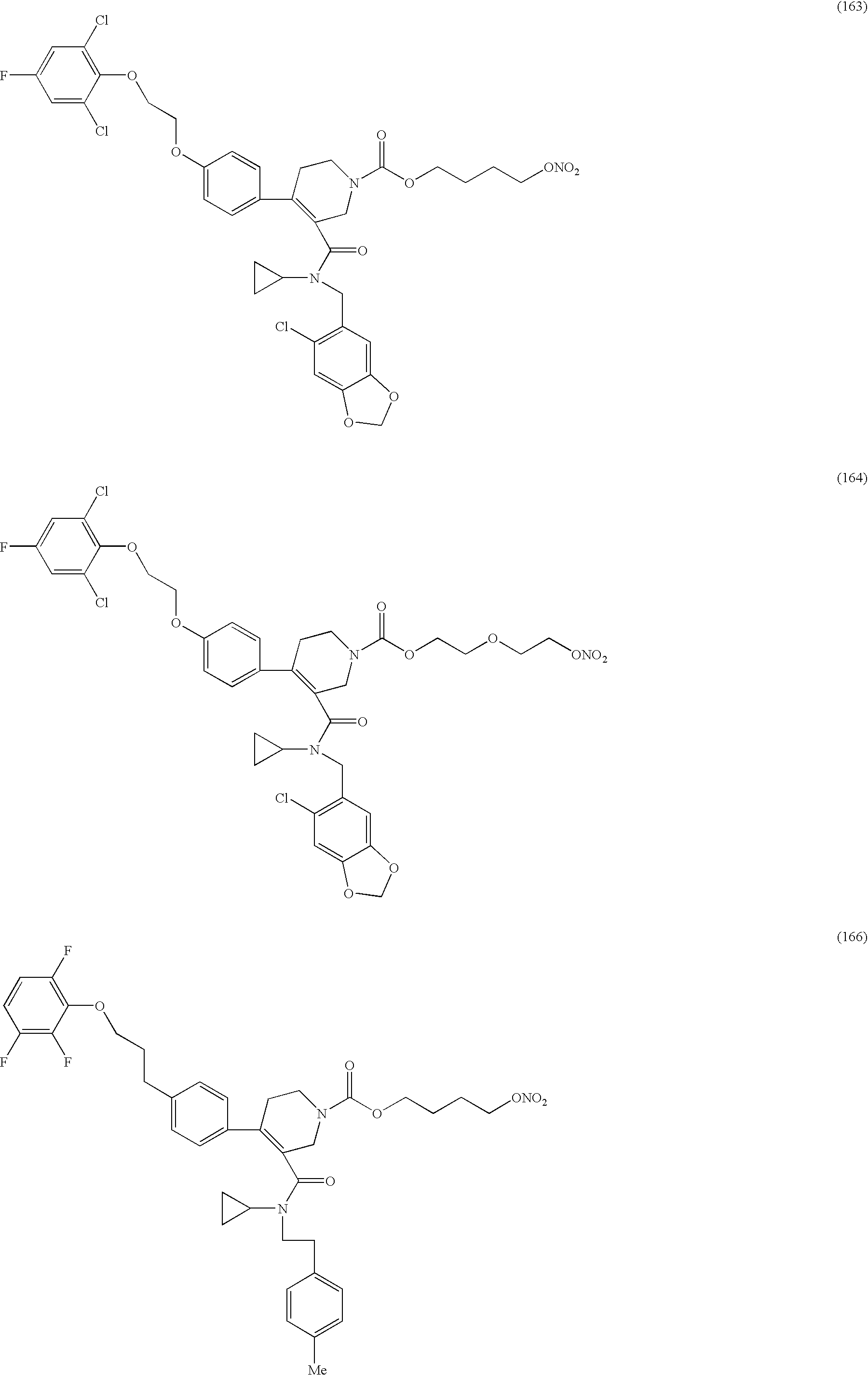

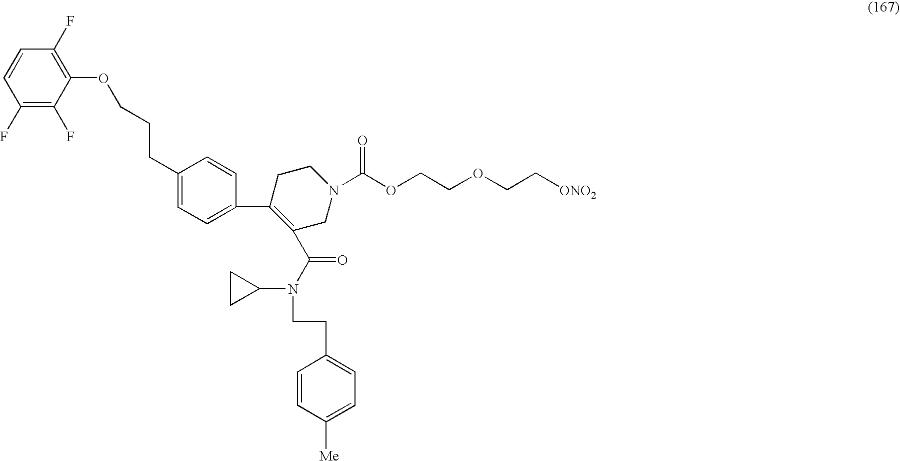

As mentioned above, object of the present invention are also pharmaceutical compositions containing at least a compound of the present invention of formula (I) together with non toxic adjuvants and/or carriers usually employed in the pharmaceutical field.

The daily dose of active ingredient that should be administered can be a single dose or it can be an effective amount divided into several smaller doses that are to be administered throughout the day. Usually, total daily dose may be in amounts preferably from 50 to 500 mg. The dosage regimen and administration frequency for treating the mentioned diseases with the compound of the invention and/or with the pharmaceutical compositions of the present invention will be selected in accordance with a variety of factors, including for example age, body weight, sex and medical condition of the patient as well as severity of the disease, route of administration, pharmacological considerations and eventual concomitant therapy with other drugs. In some instances, dosage levels below or above the aforesaid range and/or more frequent may be adequate, and this logically will be within the judgment of the physician and will depend on the disease state.

The compounds of the invention may be administered orally, parenterally, rectally or topically, by inhalation or aerosol, in formulations eventually containing conventional non-toxic pharmaceutically acceptable carriers, adjuvants and vehicles as desired. Topical administration may also involve the use of transdermal administration such as transdermal patches or iontophoresis devices. The term “parenteral” as used herein, includes subcutaneous injections, intravenous, intramuscular, intrasternal injection or infusion techniques.

Injectable preparations, for example sterile injectable aqueous or oleaginous suspensions may be formulated according to known art using suitable dispersing or wetting agents and suspending agents. The sterile injectable preparation may also be a sterile injectable solution or suspension in a non-toxic parenterally acceptable diluent or solvent. Among the acceptable vehicles and solvents are water, Ringer's solution and isotonic sodium chloride. In addition, sterile, fixed oils are conventionally employed as a solvent or suspending medium. For this purpose any bland fixed oil may be employed including synthetic mono or diglycerides, in addition fatty acids such as oleic acid find use in the preparation of injectables.

Suppositories for rectal administration of the drug can be prepared by mixing the active ingredient with a suitable non-irritating excipient, such as cocoa butter and polyethylene glycols.

Solid dosage forms for oral administration may include capsules, tablets, pills, powders, granules and gels. In such solid dosage forms, the active compound may be admixed with at least one inert diluent such as sucrose, lactose or starch. Such dosage forms may also comprise, as in normal practice, additional substances other than inert diluents, e.g. lubricating agents such as magnesium stearate. In the case of capsules, tablets and pills, the dosage forms may also comprise buffering agents. Tablets and pills can additionally be prepared with enteric coatings.

Liquid dosage forms for oral administration may include pharmaceutically acceptable emulsions, solutions, suspensions, syrups and elixirs containing inert diluents commonly used in the art, such as water. Such compositions may also comprise adjuvants, such as wetting agents, emulsifying and suspending agents, and sweetening, flavouring and the like.

The compounds of the present invention can be synthesized as follows.

Synthesis Procedure

1. The compound of general formula (I) as above defined wherein:

A¹ and j are as above defined; Xₐ is X_{b}Yₐ wherein X_{b} is —CO— and Yₐ is as above defined, can be obtained by a process comprising:

1a. reacting a compound of formula B¹ with a compound of formula (IIIa) in the molar ratio 1:1, 1:2 or 1:3 dependent on value of the integer j in the general formula (I):

B¹+HOOC—Yₐ—ONO₂ (IIIa)

wherein Yₐ is as above defined; B¹ has the same meaning as A¹ with N₁ equal to —NH— and, if in A¹ a group —O— is linked to —Xₐ—ONO₂, then in B¹ this group corresponds to —OH; in presence of a condensing agent like dicyclohexylcarbodiimide (DCC) or N,N′-carbonyldiimidazol (CDI) or other known condensing reagents such as HATU in solvent such as DMF, THF, chloroform at a temperature in the range from −5° C. to 50° C. in the presence or not of a base as for example DMAP.

The nitric acid ester compounds of formula (IIIa) can be obtained from the corresponding alcohols of formula HOOC—Yₐ—OH (IIIb), that are commercially available, by reaction with nitric acid and acetic anhydride in a temperature range from −50° C. to 0° C. or reacting the corresponding halogen derivatives of formula HOOC—Yₐ-Hal (IIIc) wherein Hal is an alogen atom preferable Cl, Br, I, that are commercially available, with AgNO₃ as already described in WO 2006/008196.

Compounds of formula B¹ wherein B¹ has formula (Ia) with N₁ equal to —NH— are known compounds and can be prepared as described in WO 2005/054244.

Compounds of formula B¹ wherein B¹ has formula (Ib) with N₁ equal to —NH— are known compounds and can be prepared as described in WO 2005/054243.

Compounds of formula B¹ wherein B′ has formula (Ic) with N₁ equal to —NH— are known compounds and can be prepared as described in WO 03/093267.

Compounds of formula B¹ wherein B¹ has formula (Id) with N₁ equal to —NH— are known compounds and can be prepared as described in WO 2005/040173.

Compounds of formula B¹ wherein B¹ has formula (Ie) with N₁ equal to —NH— are known compounds and can be prepared as described in WO 2005/040165.

Compounds of formula B¹ wherein B¹ has formula (If) with N₁ equal to —NH— are known compounds and can be prepared as described in WO 2005/040120.

Compounds of formula B¹ wherein B¹ has formula (Ig) with N₁ equal to —NH— are known compounds and can be prepared as described in WO 2004/096804.

Compounds of formula B¹ wherein B¹ has formula (Ih) with N₁ equal to —NH— are known compounds and can be prepared as described in WO 2004/096803.

Compounds of formula B¹ wherein B¹ has formula (Ii) with N₁ equal to —NH— are known compounds and can be prepared as described in WO 2004/096799.

Compounds of formula B¹ wherein B¹ has formula (Ij) with N₁ equal to —NH— are known compounds and can be prepared as described in WO 2004/096769.

Compounds of formula B¹ wherein B¹ has formula (Ik) with N₁ equal to —NH— are known compounds and can be prepared as described in WO 2004/096366.

Compounds of formula B¹ wherein B¹ has formula (Il) with N₁ equal to —NH— are known compounds and can be prepared as described in WO 2004/002957.

1b. reacting a compound of formula B¹ as above defined with a compound of formula (IIId) in the molar ratio 1:1, 1:2 or 1:3 dependent on value of the integer j in the general formula (I):

B¹+Act-CO—Yₐ—ONO₂ (IIId)

wherein Yₐ is as above defined; Act is an halogen atom or a carboxylic acid activating group used in peptide chemistry as:

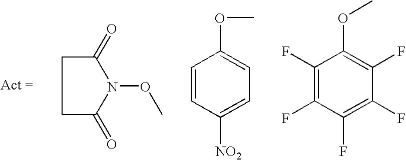

The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂.

The compounds of formula (IIId) can be obtained as described in WO 2006/008196.

1c. reacting a compound of formula A¹-(CO—Yₐ-Hal)ⱼ (IVa), wherein A¹, Yₐ and j are as above defined, with AgNO₃ as already described. Compounds (IVa) can be obtained by reacting a compound B¹ with a compound of formula (IIIc), as above defined, in the molar ratio 1:1, 1:2 or 1:3 dependent on value of the integer j in the general formula (I), with a condensing reagent such as DCC or CDI as above described.

1d. reacting a compound of formula A¹-(CO—Yₐ—OH)ⱼ (Va), wherein A¹, Yₐ and j are as above defined, with triflic anhydride/tetraalkylammonium nitrate salt in an aprotic polar/non-polar solvent such as DMF, THF or CH₂Cl₂ at temperatures range between −60° to 65° C. as already described. Compounds (Va) can be obtained by reacting a compound B¹ with a compound of formula (IIIb), as above defined, in the molar ratio 1:1, 1:2 or 1:3 dependent on value of the integer j in the general formula (I), with a condensing reagent as above described.

2. The compound of general formula (I) as above defined wherein:

A¹ and j are as above defined; Xₐ is —X_{b}—Yₐ— wherein X_{b} is —C(O)O— and Yₐ is as above defined, can be obtained by a process comprising:

2a. reacting a compound of formula B¹ with a compound of formula (VIa) in the molar ratio 1:1, 1:2 or 1:3 dependent on value of the integer j in the general formula (I):

B¹+Act-CO—O—Yₐ—ONO₂ (VIa)

wherein B¹, Act and Yₐ are as above defined.

The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂. The synthesis of compounds (VIa) is described in WO 2006/008196.

2b. reacting a compound of formula A¹-(COO—Yₐ-Hal)ⱼ (VIIa) wherein A¹, Yₐ, Hal and j are as above defined, with AgNO₃ as above described.

The compounds of formula (VIIa) can be obtained by reacting a compound B¹ with a compound of formula Act-CO—O—Yₐ-Hal (VIIb) in the molar ratio 1:1, 1:2 or 1:3 dependent on value of the integer j in the general formula (I).

The reaction is generally carried out in presence of an inorganic or organic base in an aprotic polar/non-polar solvent such as DMF, THF or CH₂Cl₂ at temperatures range between 0°-65° C. as above described.

Compounds (VIIb) are commercially available or can be synthesized as already described in WO 2006/008196.

1. A compound of general formula (I) or a pharmaceutically acceptable salt or stereoisomer thereof:
A¹-(Xₐ—ONO₂)ⱼ (I)
wherein:
j is an integer equal to 1, 2, or 3;
A¹ is selected from the group consisting of formula (Ia), (Ib), (Ic), (Id), (Ie), (If), (Ig), (Ih), (Ii), (Ij), (Ik) and (Il): 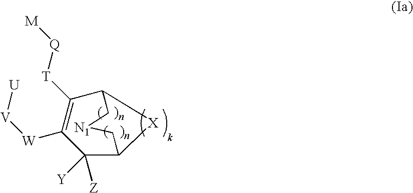
wherein:
—N₁— is a nitrogen atom bound to one group —Xₐ—ONO₂;
Y and Z represent independently from each other hydrogen (H); —F; -Me group; or Y and Z may together form a cyclopropyl ring; in case k represents the integer 1, Y and Z both represent hydrogen;
X represents —(CH₂)ₘN₁(L)(CH₂)ₘ—; —CH₂CH(K)CH₂—; —CH₂CH₂—; —CH₂OCH₂—; —CH₂SCH₂—; —CH₂SOCH₂—; —CH₂SO₂CH₂—; —CON(L)CO—; CON(L)CHR⁶—; —CHR⁶—N(L)CO—;
W represents a six-membered, non benzofused, phenyl or heteroaryl ring, substituted by V in position 3 or 4;
V represents a bond; —(CH₂)ᵣ—; -A-(CH₂)ₛ—; —CH₂-A-(CH₂)ₜ—; (CH₂)-A-; —(CH₂)₂-A-(CH₂)u-; -A-(CH₂)ᵥ—B—; —(CH₂)₃-A-CH₂—; A-(CH₂)₂—B—CH₂—; —CH₂-A-(CH₂)₂—B—; —(CH₂)3-A-(CH₂)₀—; —(CH₂)4-A-CH₂—; -A-(CH₂)₂—B—(CH₂)₂—; —CH₂-A-CH₂—CH₂—B—CH₂—; —CH₂-ACH₂—CH2-CH₂—B—; —CH₂—CH₂-A-CH₂—CH₂—B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂ CH₂—O—; —O—CH₂—C(CH₃)₂—O—; —O—C(CH₃)₂—CH₂—O—; —O—CH₂—CH(CH₃)₂O—; —O—CH(CH₃)—CH₂—O—; —O—CH₂—C(CH₂—CH₂)—O—; —O—C(CH₂—CH₂)—CH₂—O—;
A and B represent —O—; —S—; —SO—; —SO₂—;
U represents aryl; heteroaryl;
T represent —CONR¹; —(CH₂)ₚOCO—; —(CH₂)ₚN(R¹)CO—; —(CH₂)ₚN(R¹)SO₂—; —COO—;
Q represents lower alkylene; lower alkenylene;
M represents aryl-O(CH₂)vR⁵; heteroaryl-O(CH₂)vR⁵; aryl-O(CH₂)ᵥO(CH₂)wR⁵; heteroaryl-(CH₂)vO(CH₂)_{w}R⁵; aryl-OCH₂CH(R⁷)CH₂R⁵; heteroaryl-OCH₂CH(R⁷)CH₂R⁵; wherein heteroayl means preferably a lower alkyl substituted pyridyl;
L represent —R³; —COR³; —CO₂R³; —CONR²R³; —SO₂R³; —SO₂NR²R³; COCH(aryl)₂;
K represents hydrogen; —CH₂OR³; —CH₂NR²R³; —CH²NR²COR³; CH₂NR²SO₂R³; —CO₂R³; —CH₂OCONR²R³; —CONR²R³; —CH₂NR²COR²R³; CH₂SO2NR²R³; —CH₂SR³; —CH₂SOR³; —CH₂SO₂R³;
R₁ represents hydrogen, lower alkyl, lower alkenyl, lower alkynyl, cycloalkyl, aryl, cycloalkyl-lower alkyl;
R² and R^{2′} independently represent hydrogen, lower alkyl; lower alkenyl, cycloalkyl, cycloalkyl-lower alkyl;
R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di-or trisubstituted with hydroxy, —OCOR², —COOR², lower alkoxy, cyano, —CONR²R^{2′}, CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; COOR²; —CONH₂;
R⁵ represents —O—; —OH, lower alkoxy, —OCOR², —CO2R², NR²R^{2′}, OCONR²R^{2′}, NCONR²R^{2′}, cyano, —CONR²R^{2′}, SO₃H, —SONR²R^{2′}; —CO-morpholin-4-yl, —CO-((4-loweralkyl)piperazin-1-yl), —NH(NH)NH₂, NR⁴R^{4′} with the proviso that a carbon atom is attached at the most to one heteroatom in case this carbon atom is sp³-hybridized; when R⁵ is —O—, it is bound to one group —Xₐ—ONO₂;
R⁶ represents hydrogen, lower alkyl; lower alkoxy; whereby these groups may be unsubstituted or monosubstituted with hydroxy, —CONH₂, —COON, imidazolyl,—NH₂,—CN, —NH(NH)NH₂;
R⁷ represents —O—; —OH, OR², OCOR², OCOOR²; or R⁶ and R⁵ form together with the carbon atoms to which they are attached a 1,3-dioxolane ring which is substituted in position 2 with R² and R^{2′}; or R⁶ and R⁵ form together with the carbon atoms to which they are attached a 1,3-dioxolan-2-one ring; when R⁷ is —O—, it is bound to one group —Xₐ—ONO₂;
k represents the integer 0 or 1;
m and n represent the integer 0 or 1 with the proviso that in case m represents the integer 1, n is the integer 0; in case n represents the integer 1, m is the integer 0; in case k represents the integer 0, n represents the integer 0; in case X does not represent —CH₂)ₘ—N(L)-(CH₂)ₘ—, n represents the integer 0;
p, t and v independently represent the integer 1, 2, 3 or 4;
r represents the integer 1, 2, 3, 4, 5 or 6;
s represents the integer 1, 2, 3, 4 or 5;
u represents the integer 1, 2 or 3;
w represents the integer 1 or 2; 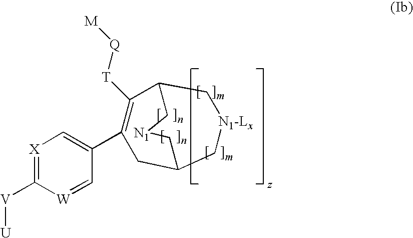
wherein
—N₁— is a nitrogen atom bound to one group —Xₐ—ONO₂;
X and W represent a nitrogen atom or a —CH— group;
V represents —(CH₂)r; -A-(CH₂)ₛ; —CH₂-A-(CH₂)t-; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂-; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;
A and B represent independently —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene or alkenylene;
M represents aryl-O(CH₂)vR⁷—; heteroaryl-O(CH₂)vR⁷; aryl-O(CH₂)vO(CH₂)wR⁷; heteroaryl-(CH₂)vO(CH₂)wR⁷; aryl-OCH₂CH(R⁶)CH₂R⁵—; heteroaryl-OCH₂CH(R⁶)CH₂R⁵—;
L when x is 1 represents —R³; —COR³; —COOR³; —CONR²R³; —SO2R³; —SO2NR²R³; COCH(Aryl)₂;
R¹ represents hydrogen; lower alkyl; lower alkenyl; or lower alkynyl; cycloalkyl; aryl; cycloalkyl-lower alkyl;
R² and R^{2′} independently represent hydrogen, lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di-or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; COOR²; —CONH₂;
R⁵ represents —O—; —OH; lower alkoxy; —OCOR²; —COOR²; —NR²R^{2′}; —OCONR²R^{2′}; —NCONR²R^{2′}; cyano; —CONR²R^{2′}; —SO₃H; —SONR²R^{2′}; —CO-morpholin-4-yl; —CO-(4-loweralkyl)piperazin-1-yl); —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;
R⁶ represents —O—; —OH; OR²; OCOR²; OCOOR²; or R⁶ and R⁵ form together with the carbon atoms to which they are attached a 1,3-dioxolane ring which is substituted in position 2 with R² and R^{2′}; or R⁶ and R⁵ form together with the carbon atoms to which they are attached a 1,3-dioxolan-2-one ring; when R⁶ is —O—, it is bound to one group —Xₐ—ONO₂;
R⁷ represents lower alkoxy;
m and n represent the integer 0 or 1, with the proviso that in case m represents the integer 1, n is the integer 0, and in case n represents the integer 1, m is the integer 0; p, t, and v represent the integer 1, 2, 3 or 4;
r represents the integer 3, 4, 5 or 6;
s represents the integer 2, 3, 4 or 5;
u represents the integer 1, 2 or 3;
w represents the integer 1 or 2;
x and z represent the integer 0 or 1; 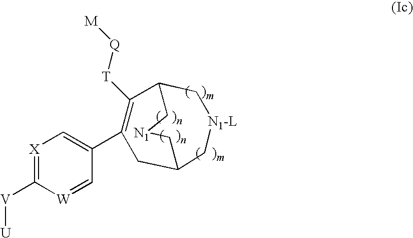
wherein:
—N1- is a nitrogen atom bound to one group —Xₐ—ONO₂;
X and W represent a nitrogen atom or a —CH— group;
V represents —(CH₂)r; -A-(CH₂)s; —CH₂-A-(CH₂)t-; —(CH₂)s-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)4-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;
A and B represent independently —O—; —S—; —SO—; —SO₂—;
U represents aryl; heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene; lower alkenylene;
M represents hydrogen; cycloalkyl; aryl; heterocyclyl; heteroaryl;
L represents —R³; —COR³; —COOR³; —CONR²R³; —SO2R³; —SO2NR²R³; COCH(Aryl)₂;
R¹ represents hydrogen; lower alkyl; lower alkenyl; lower alkynyl; cycloalkyl; aryl; cycloalkyl-lower alkyl;
R² and R^{2′} independently represent hydrogen, lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl;
heteroaryloxy-lower alkyl, whereby these groups may be unsubstituted, or mono, di-or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; —COOR²; —CONH₂;
m and n represent the integer 0 or 1, with the proviso that in case m represents the integer 1, n is the integer 0, and in case n represents the integer 1, m is the integer 0;
p and t represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4; 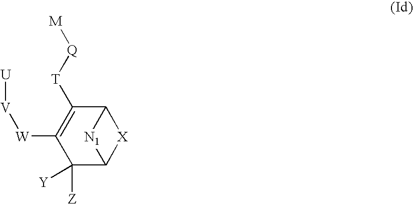
wherein:
—N₁— is a nitrogen atom bound to one group —Xₐ—ONO₂;
Y, Z represent independently hydrogen, F, or o methyl group; or Y and Z may together form a cyclopropyl ring;
X represents —CH₂CH(K)CH₂—, —CH₂CH₂—, —CH₂OCH₂—; —CH₂SCH₂—, —CH₂SOCH₂—, —CH₂SO2CH₂—, —CO—NL-CHR⁶—; —CHR⁶—NL-CO—;
W represents a six membered non benzofused phenyl, or heteroaryl ring substituted by V in position 3 or 4;
V represents a bond, represents —(CH₂)ᵣ; -A-(CH₂)ₛ; —CH₂-A-(CH₂)t-; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)4-A-CH₂—; -A-CH₂CH₂—B—CH₂—CH₂—; —CH₂-A-CH₂CH₂—, —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(OCH₃)₂—CH₂—O—; —O—CH₂—C(CH₃)₂—CH₂—O—; —O—CH₂—C(CH₃)₂—O—; —OC(CH₃)₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;
A and B independently represent —O—, —S—, —SO—, —SO₂—;
U represents aryl, heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene, lower alkenylene; M represents aryl-O(CH₂)vR⁵, heteroaryl-O(CH₂)ᵥR⁵, aryl-OCH₂CH₂O(CH₂)_{w}R⁵; heteroaryl-OCH₂CH₂O(CH₂)wR⁵;
L represents —R³; —COR³; —COOR³; —CONR₂R³; —SO₂R³; —SO₂NR₂R³; COCH(Aryl)₂;
K represents hydrogen; —CH₂OR³; —CH₂NR₂R³; —CH₂NR₂COR³; —CH₂NR₂SO₂R³; —CO₂R³; —CH₂OCONR₂R³; —CONR₂R³; —CH₂NR₂COR₂R³; —CH₂SO₂NR₂R³; —CH₂SR³; —CH₂SOR³; —CH₂SO₂R³;
R¹ represents hydrogen, lower alkyl, lower alkenyl, lower alkynyl, cycloalkyl, aryl, cycloalkyl-lower alkyl;
R₂ and R₂′ independently represent hydrogen, lower alkyl, lower alkenyl, cycloalkyl, cycloalkyl-lower alkyl;
R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di-or trisubstituted with hydroxy, —OCOR₂, —COOR₂, lower alkoxy, cyano, —CONR₂R_{2′}, —CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; —COOR₂; —CONH₂;
R⁵ represents —O—; —OH, —OCOR₂, —CO₂R₂, —NR₂R²′, —OCONR₂R₂′, NCONR₂R₂′, cyano, —CONR₂R²′, SO3H, —SONR₂R₂′; —CO-morpholin-4-yl, —CO-((4-loweralkyl)piperazin-1-yl), —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;
R⁶ represents hydrogen, lower alkyl; lower alkoxy; whereby these groups may be unsubstituted or monosubstituted with hydroxy, —CONH₂, —COOH, imidazolyl,—NH₂,—CN, —NH(NH)NH₂;
p and t independently represent the integer 1, 2, 3 or 4;
r represents the integer 1, 2, 3, 4, 5 or 6;
s represents the integer 1, 2, 3, 4 or 5;
u represents the integer 1, 2 or 3;
v represents the integer 2, 3 or 4; w represents the integer 1 or 2; 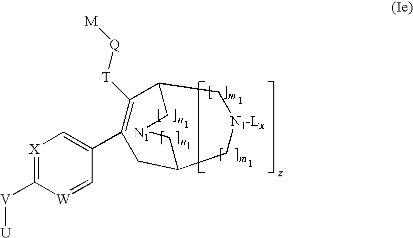
wherein:
—N1- is a nitrogen atom bound to one group —Xₐ—ONO₂;
X and W represent a nitrogen atom or a —CH— group;
V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;
A and B represent independently —O—; —S—; —SO—; —SO₂—;
U represents aryl; heteroaryl;
T represents —CONR¹—; —(CH₂)ₚOCO—; (CH₂)ₚN(R¹)CO—; —(CH₂)ₚN(R¹)SO₂—; —CO₂—;
Q represents lower alkylene or alkenylene;
M represents aryl-O(CH₂)ᵥR⁵—; heteroaryl-O(CH₂)vR⁵; aryl-O(CH₂)₂O(CH₂)_{w}R⁵; heteroaryl-(CH₂)₂O(CH₂)_{w}R⁵;
L represents —R³; —COR³; —COOR³; —CONR₂R³; —SO₂R³; —SO₂NR₂R³; COCH(Aryl)₂;
R¹ represents hydrogen; lower alkyl; lower alkenyl; or lower alkynyl; cycloalkyl; aryl; cycloalkyl-lower alkyl; R₂ and R₂′ independently represent hydrogen, lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di-or trisubstituted with hydroxy; —OCOR₂, —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; —COOR₂; —CONH₂;
R⁵ represents —O—; —OH; —OCOR²; —COOR²; —NR²R^{2′}; —OCONR²R^{2′}; —NCONR²R^{2′}; cyano; —CONR²R^{2′}; —SO₃H; —SONR²R^{2′}; —CO-morpholin-4-yl; —CO-((4-loweralkyl)piperazin-1-yl); —NH(NH)NH₂, —NR⁴R^{4′} with the proviso that a carbon atom is attached at the most to one heteroatom in case this carbon atom is sp³-hybridized; when R⁵ is —O—, it is bound to one group —Xₐ—ONO₂;
m1 and n1 represent the integer 0 or 1, with the proviso that in case m₁ represents the integer 1,
n₁ is the integer 0, and in case n₁ represents the integer 1, m₁ is the integer 0;
p, t, represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4;
w represents the integer 1 or 2;
x represents the integer o or 1;
z represents the integer 0 or 1; if z is the integer 0, n₁ is the integer 1; 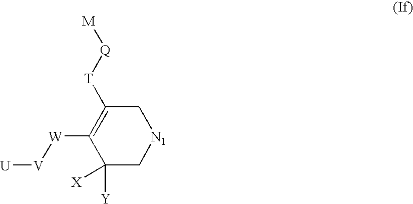
wherein:
—N1- is a nitrogen atom bound to one group —Xₐ—ONO₂;
X and Y independently represent hydrogen, —F, or a methyl group; X and Y do not represent both hydrogen at the same time or X and Y may together form a cyclopropyl ring;
W represents a phenyl or heteroaryl ring, the heteroaryl ring being a six-membered and non-fused ring, the phenyl ring and the heteroaryl ring are substituted with V in position 3 or 4;
V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂₋B—; —CH₂—CH₂-A-CH₂CH₂B—; —O—CH₂—CH(OCH₃)—CH₂—O—; —O—CH₂₋CH(CH₃)—CH₂—O—;—O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;
U represents aryl, heteroaryl;
A and B independently represent —O—, —S—, —SO—, —SO₂—;
T represents —CONR¹—; —(CH₂)ₚOCO—; —(CH₂)ₚN(R¹)CO—; —(CH₂)ₚN(R¹)SO₂—; —CO₂—; —(CH₂)ₚOCO—NR¹; —(CH₂)ₚN(R₂)CO—NR¹—
R¹ and R₂ independently represent hydrogen, lower alkyl, lower alkenyl, lower alkynyl, cycloalkyl, aryl-lower alkyl; heteroaryl-lower alkyl; cycloalkyl-lower alkyl;
Q represents lower alkylene, lower alkenylene;
M represents hydrogen; cycloalkyl; aryl; heterocyclyl or heteroaryl;
p and t independently represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4; 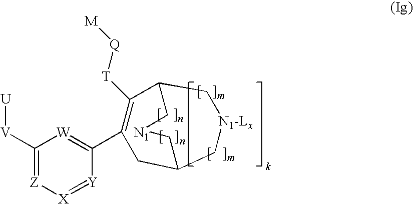
wherein:
—N₁— is a nitrogen atom bound to one group —Xₐ—ONO₂;
Z, Y, X and W represent independently a nitrogen atom, or a —CH— group; at least two of the Z, Y, X and W represent a —CH— group;
V represents a bond; —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂CH₂—CH₂—B—; —CH₂—CH₂-A-CH₂CH₂B—;
A and B represent independently —O—; —S—; —SO—; —SO₂—; U represents aryl; heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene or alkenylene;
M represents hydrogen; cycloalkyl; aryl; heteroaryl; heterocyclyl;
L when k is 1 represents —R³; —COR³; —COOR³; —CONR²R³; —SO₂R³; —SO₂NR²R³; COCH(Aryl)₂;
R¹ represents hydrogen; lower alkyl; lower alkenyl; or lower alkynyl; cycloalkyl; aryl; cycloalkyl-lower alkyl;
R² and R^{2′} independently represent hydrogen, lower alkyl; lower alkenyl; cycloalkyl; cycloalkyl-lower alkyl;
R³ represents hydrogen; lower alkyl; lower alkenyl; cycloalkyl; aryl; heteroaryl; heterocyclyl; cycloalkyl-lower alkyl; aryl-lower alkyl; heteroaryl-lower alkyl; heterocyclyl-lower alkyl; aryloxy-lower alkyl; heteroaryloxy-lower alkyl, whereby these groups may be unsubstituted, or mono, di-or trisubstituted with hydroxy; —OCOR²; —COOR²; lower alkoxy; cyano; —CONR²R^{2′}; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; —COOR²; —CONH₂;
k represents the integer 0 or 1;
m and n represent the integer 0 or 1, with the proviso that in case m represents the integer 1, n is the integer 0, and in case n represents the integer 1, m is the integer 0;
p and t represent the integer 1, 2, 3 or 4;
r represents the integer 1, 2, 3, 4, 5 or 6;
s represents the integer 1, 2, 3, 4 or 5;
u represents the integer 1, 2 or 3;
v represents the integer 2, 3 or 4; 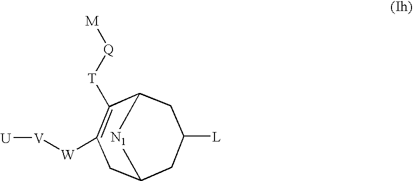
wherein:
—N1- is a nitrogen atom bound to one group —Xₐ—ONO₂;
W is a six-membered non benzofused phenyl, or heteroaryl ring substituted by V in position 3 or 4;
V represents a bond, represents —(CH₂)ᵣ; -A-(CH₂)ₛ; —CH₂-A-(CH₂)ₜ-; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;
A and B independently represent —O—, —S—, —SO—, —SO₂—;
U represents aryl, heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene, lower alkenylene;
M represents hydrogen; cycloalkyl; aryl, hetrocyclyl; heteroaryl; aryl-O(CH₂)ᵥR⁵, heteroaryl-O(CH₂)ᵥR⁵, aryl-OCH₂CH₂O(CH₂)_{w}R⁵; heteroaryl-OCH₂CH₂O(CH₂)_{w}R⁵;
L represents hydrogen; —CH₂OR³; —CH₂NR²R³;—CH₂—NR²COR³; —CH₂NR²SO₂R³; —COOR³; —CH₂OCONR²R³; —CONR²R³; —CH₂NR²CONR²R³; —CH₂SO₂NR²R³; —CH₂SR³; —CH₂SOR³; —CH₂SO₂R³;
R¹ represents hydrogen; lower alkyl; lower alkenyl; lower alkynyl; cycloalkyl; aryl; cycloalkyl-lower alkyl; R² and R^{2′} represent independently hydrogen; lower alkyl; lower alkenyl; cycloalkyl; cycloalkyl-lower alkyl;
R³ represents hydrogen, lower alkyl; lower alkenyl, cycloalkyl, aryl, heteroaryl, heterocyclyl, cycloalkyl-lower alkyl, aryl-lower alkyl, heteroaryl-lower alkyl; heterocyclyl-lower alkyl; aryloxy-lower alkyl, heteroaryloxy-lower alkyl, whereby these groups may be unsubstituted, or mono, di-or trisubstituted with hydroxy, —OCOR², —COOR², lower alkoxy, cyano, —CONR²R^{2′}, —CO-morpholin-4-yl, —CO-((4-loweralkyl)piperazin-1-yl), —NH(NH)NH₂, —NR⁴R^{4′} or lower alkyl with the proviso that a carbon atom is attached at the most to one heteroatom in case this carbon atom is sp³-hybridized;
R⁴ and R^{4′} independently represent hydrogen; lower alkyl; cycloalkyl; cycloalkyl-lower alkyl; hydroxy-lower alkyl; —COOR²; —CONH₂;
R⁵ represents —O—; —OH, —OCOR², —CO₂R², —NR²R^{2′}, —OCONR²R^{2′}, —NCONR²R^{2′}, cyano, —CONR²R^{2′}, SO₃H, —SONR²R^{2′}; —CO-morpholin-4-yl, —CO-((4-loweralkyl)piperazin-1-yl), —NH(NH)NH₂, —NR⁴R^{4′}with the proviso that a carbon atom is attached at the most to one heteroatom in case this carbon atom is sp³-hybridized; when R⁵ is —O—, it is bound to one group —Xₐ—ONO₂;
p and t independently represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4;
w represents the integer 1 or 2; 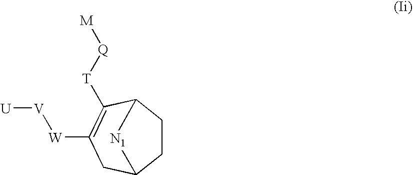
wherein:
—N₁— is a nitrogen atom bound to one group —Xₐ—ONO₂;
W is a six-membered non benzofused phenyl, or heteroaryl ring substituted by V in position 3 or 4; V represents a bond, represents —(CH₂)ᵣ; -A-(CH₂)ₛ; —CH₂-A-(CH₂)ₜ—; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF3)-CH₂—O—; —O—CH₂₋C(CH₃)₂—CH₂—O—; —O—CH₂—C(CH₃)₂—O—; —O—C(CH₃)₂—CH₂—O—; —O—CH₂—CH(CH₃)—O—; —O—(CH₃)CH₂—O—; —O—CH₂—C(CH₂CH₂)—O—; —O—C(CH₂CH₂)—CH₂—O—;
A and B independently represent —O—, —S—, —SO—, —SO₂—;
U represents aryl, heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene, lower alkenylene;
M represents hydrogen; cycloalkyl; aryl; heterocyclyl; heteroaryl;
R¹ represents hydrogen, lower alkyl, lower alkenyl, lower alkynyl, cycloalkyl, aryl, cycloalkyl-lower alkyl;
p and t independently represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4; 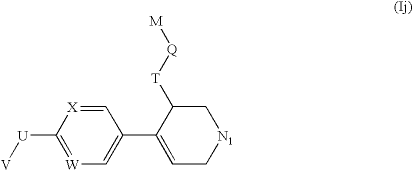
wherein:
—N₁— is a nitrogen atom bound to one group —Xₐ—ONO₂;
X and W represent a nitrogen atom or a —CH— group;
V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—;
A and B represent independently —O—; —S—; —S(O)—; —S(O)₂—;
U represents aryl; heteroaryl;
T represents —CONR¹—; —(CH₂)ₚOCO—; —(CH₂)ₚN(R¹)CO—; —(CH₂)ₚN(R¹)SO₂—; —CO₂—; —(CH₂)ₚOCONR¹—; —(CH₂)ₚN(R^{1′})CONR¹—;
Q represents lower alkylene; lower alkenylene;
M represents hydrogen, cycloalkyl; aryl; heterocyclyl; heteroaryl; aryl-O(CH₂)ᵥR²—; heteroaryl-O(CH₂)ᵥR²; aryl-O(CH₂)₂O(CH₂)_{w}R²; heteroaryl-(CH₂)₂O(CH₂)_{w}R²; aryl-OCH₂CH(R⁵)CH₂R²; heteroaryl-OCH₂CH(R⁵)CH₂R²;
R¹ and R^{1′} independently represent hydrogen; lower alkyl; lower alkenyl; or lower alkynyl; cycloalkyl; aryl; cycloalkyl-lower alkyl;
R² represents —O—; —OH; lower alkoxy; —OCOR³; —COOR³; —NR³R^{3′}; —OCONR³R^{3′}; —NCONR³R^{3′}; cyano; —CONR³R³; —SO₃H; —CO-morpholin-4-yl; —CO-((4-loweralkyl)piperazin-1-yl); —NH(NH)NH₂, —NR⁴R^{4′} or lower alkyl with the proviso that a carbon atom is attached at the most to one heteroatom in case this carbon atom is sp³-hybridized; when R₂ is —O—, it is bound to one group —Xₐ—ONO₂;
R³ and R^{3′} represent independently hydrogen; lower alkyl; lower alkenyl; cycloalkyl; cycloalkyl-lower alkyl;
R⁴ and R^{4′} independently represent hydrogen; lower alkyl; cycloalkyl; cycloalkyl-lower alkyl; hydroxy-lower alkyl; COOR₂; —CONH₂;
R⁵ represents —O—; —OH; —OR²; —OCOR²; —OCOOR²; or R⁵ and R² form together with the carbon atoms to which they are attached a 1, 3 dioxalane ring which is substituted in position 2 with R³ and R^{3′}; or R⁵ and R² form together with the carbon atoms to which they are attached a 1, 3 dioxola-2-one ring; when R⁵ is —O—, it is bound to one group —Xₐ—ONO₂;
p, t, represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4;
w represents the integer 1 or 2; 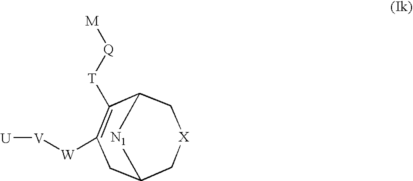
wherein:
—N₁— is a nitrogen atom bound to one group —Xₐ—ONO₂;
X represents —O—, —S—, —SO—, —SO₂—;
W is a six-membered non benzofused phenyl, or heteroaryl ring substituted by V in position 3 or 4;
V represents a bond, represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—CH₂—B—; —CH₂—CH₂-A-CH₂CH₂B—; —O—CH₂—CH(OCH₃)—CH₂—O—; —O—CH₂—CH(CH₃)—CH₂—O—; —O—CH₂—CH(CF₃)—CH₂—O—; —O—CH₂—C(CH3)₂—CH₂—O—; —O—CH₂—C(CH₃)₂—O—; —O—C(CH₃)₂—CH₂—O—; —O—CH₂—CH(CH₃)—O—; —O—CH(CH₃)CH₂—O—; —O—CH₂—C(CH₂CH₂)—O—; —O—C(CH₂CH₂)—CH₂—O—;
A and B independently represent —O—, —S—, —SO—, —SO₂—;
U represents aryl, heteroaryl;
T represents —CONR¹—; —(CH₂)ₚOCO—; —(CH₂)ₚN(R¹)CO—; —(CH₂)ₚN(R¹)SO₂—; —CO₂—;
Q represents lower alkylene, lower alkenylene;
M represents hydrogen, cycloalkyl, aryl; heterocycyl; heteroaryl;
R¹ represents hydrogen, lower alkyl, lower alkenyl, lower alkynyl, cycloalkyl, aryl, cycloalkyl-lower alkyl;
p and t independently represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4; 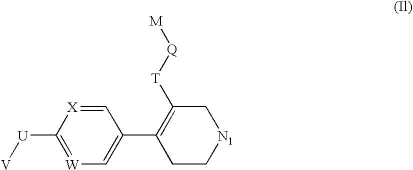
wherein:
—N₁— is a nitrogen atom bound to one group —Xₐ—ONO₂;
X and W represent independently a nitrogen atom or a —CH— group;
V represents —(CH₂)ᵣ; -A-(CH₂)ₛ; —CH₂-A-(CH₂)ₜ—; —(CH₂)ₛ-A-; —(CH₂)₂-A-(CH₂)ᵤ—; -A-(CH₂)ᵥ—B—; —(CH₂)₃-A-CH₂—; -A-CH₂CH₂—B—CH₂—; —CH₂-A-CH₂CH₂—B—; —(CH₂)₃-A-CH₂—CH₂—; —(CH₂)₄-A-CH₂—; -A-CH₂CH₂—B—CH₂—CH₂—; —CH₂-A-CH₂CH₂—B—CH₂—; —CH₂-A-CH₂—CH₂—B—; —CH₂—CH₂-A-CH₂CH₂B—;
A and B independently represent —O—, —S—, —SO—, —SO₂—;
U represents aryl, heteroaryl;
T represents —CONR¹—; —(CH₂)ₚOCO—; —(CH₂)ₚN(R¹)CO—; —(CH₂)ₚN(R¹)SO₂—; —CO₂—; —(CH₂)ₚOCONR¹—; —(CH₂)ₚN(R¹′)CONR¹—;
Q represents lower alkylene, lower alkenylene;
M represents hydrogen; cycloalkyl; aryl; heterocycyl; heteroaryl;
R¹ and R¹′ represent independently hydrogen; lower alkyl; lower alkenyl; lower alkynyl; cycloalkyl; aryl; cycloalkyl-lower alkyl;
p and t independently represent the integer 1, 2, 3 or 4;
r represents the integer 3, 4, 5 or 6;
s represents the integer 2, 3, 4 or 5;
u represents the integer 1, 2 or 3;
v represents the integer 2, 3 or 4;
Xₐ is equal to —X_{b}—Yₐ— wherein X_{b} is —CO— or —COO—;
Yₐ is a bivalent radical having the following meaning: a)
straight or branched C1-C₂O alkylene, preferably C1-C10, being optionally substituted with one or more of the substituents selected from the group consisting of: halogen atoms, hydroxy, —ONO₂ or Tₐ, wherein Tₐ is —OC(O)(C₁-C₁₀ alkyl)-ONO₂ or —O(C₁-C₁₀ alkyl)-ONO₂;
-cycloalkylene with 5 to 7 carbon atoms into cycloalkylene ring, the ring being optionally substituted with side chains T_{b}, wherein T_{b} is straight or branched alkyl with from 1 to 10 carbon atoms, preferably CH₃;
b) 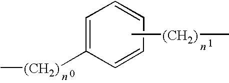
c) 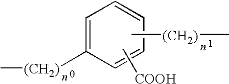
wherein n⁰ is an integer from 0 to 20, and n¹ is an integer from 1 to 20;
d) 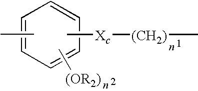
wherein:
n¹ is as defined above and n² is an integer from 0 to 2;
X_{c}══—OCO— or —COO— and R₂ is H or CH₃;
e) 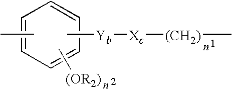
wherein:
n¹, n²,R₂ and X_{c} are as defined above;
Y_{b} is —CH₂—CH₂— or —CH═CH—(CH₂)n²-;
f) 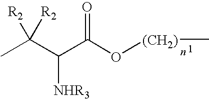
wherein:
n¹ and R₂ are as defined above, R₃ is H or —COCH₃;
with the proviso that when Ya is selected from the bivalent radicals mentioned under b)-f), the —ONO₂ group is linked to a —(CH₂)n¹ group;
g) 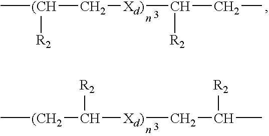
wherein X_{d} is —O— or —S—, n³ is an integer from 1 to 6, preferably from 1 to 4, R₂ is as defined above;
h) 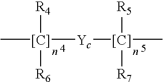
wherein:
n⁴ is an integer from 0 to 10;
n⁵ is an integer from 1 to 10;
R₄, R₅, R₆, R₇ are the same or different, and are H or straight or branched C₁-C₄ alkyl, preferably R₄, R₅, R₆, R₇are H;
wherein the —ONO₂ group is linked to 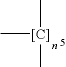
wherein n⁵ is as defined above;
Y_{c} is an heterocyclic saturated, unsaturated or aromatic 5 or 6 members ring, containing one or more heteroatoms selected from nitrogen, oxygen, sulfur, and is selected from the group consisting in:

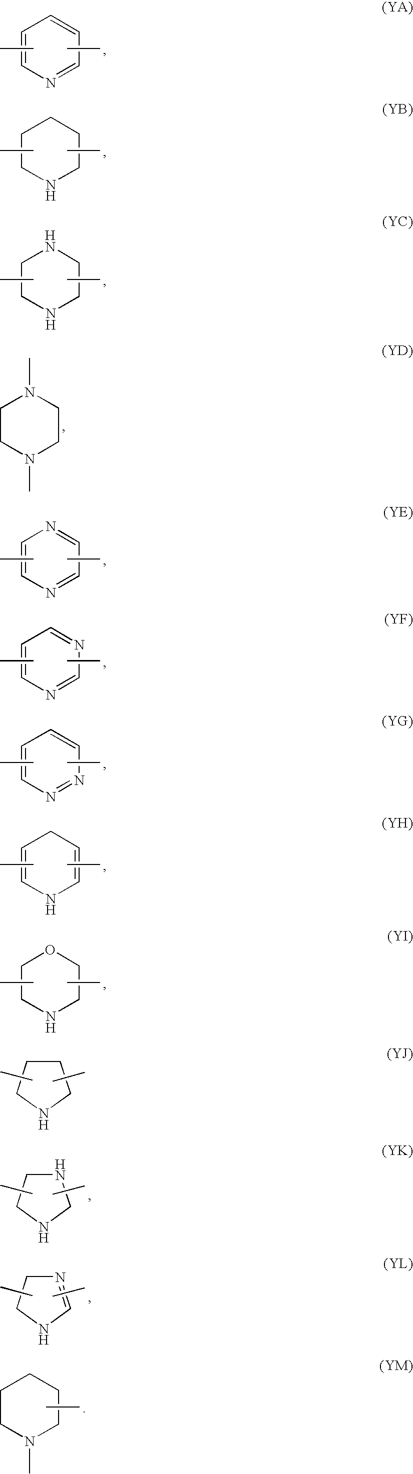

2. A compound of general formula (I) or a pharmaceutically acceptable salt or stereoisomer thereof according to claim 1, wherein Ya is a bivalent radical having the following meaning:
a)
-straight or branched C₁-C₁₀ alkylene;
b) 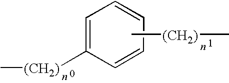
wherein n⁰ is 0 or 1, n¹ is 1;
with the proviso that the —ONO₂ group is linked to —(CH₂)n¹group;
g) 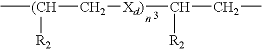
wherein X_{d} is —O— or —S—, n³ is 1 and R₂ is H.

3. A compound according to claim 1, selected from the group consisting of:

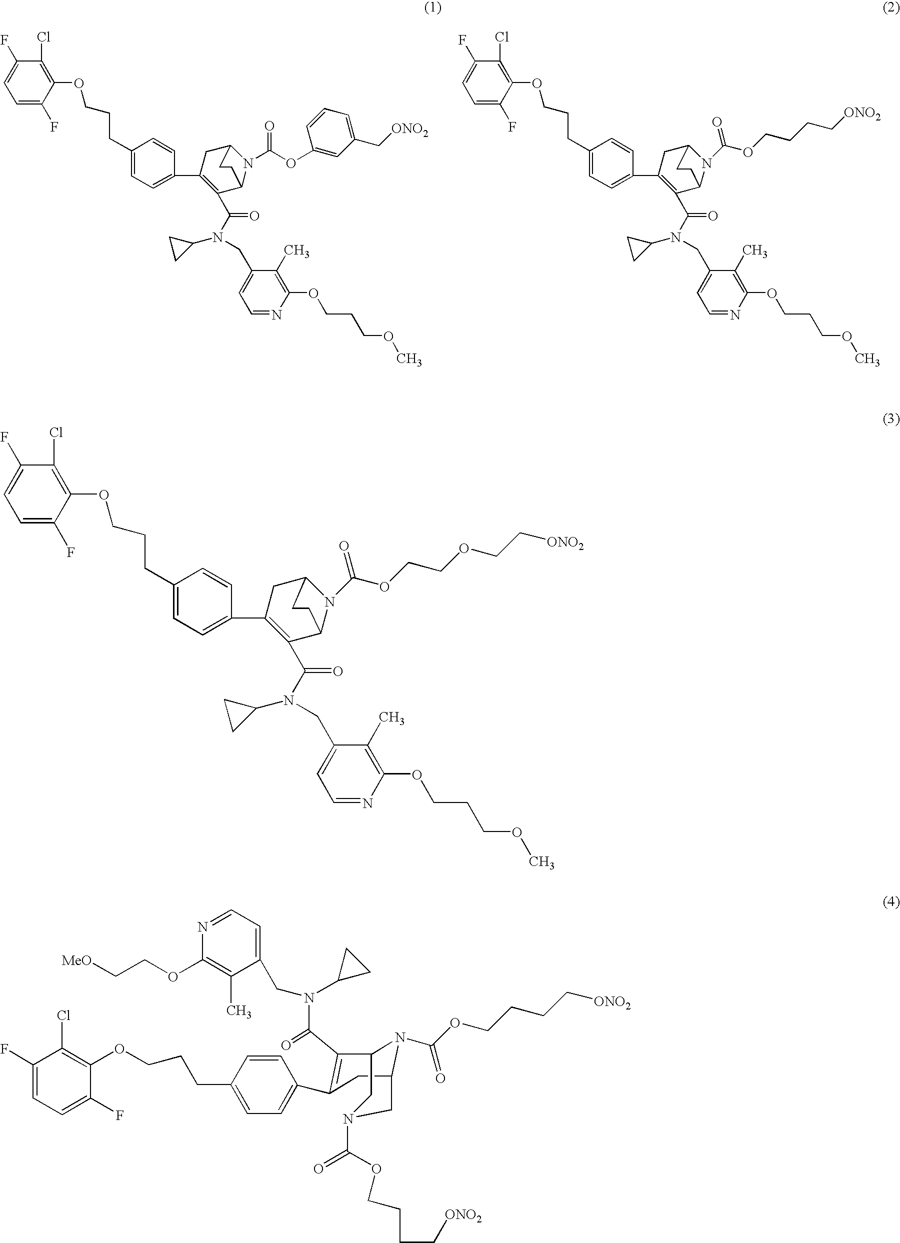

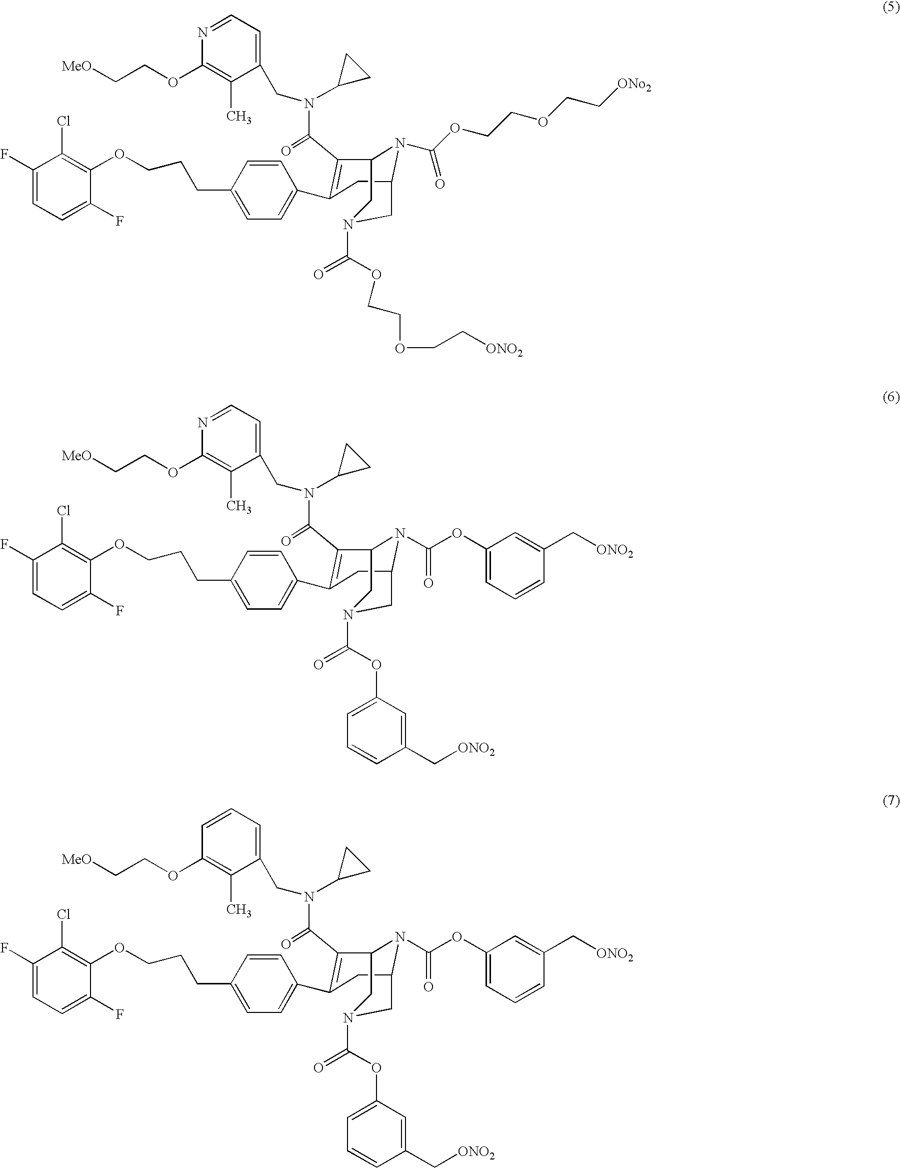

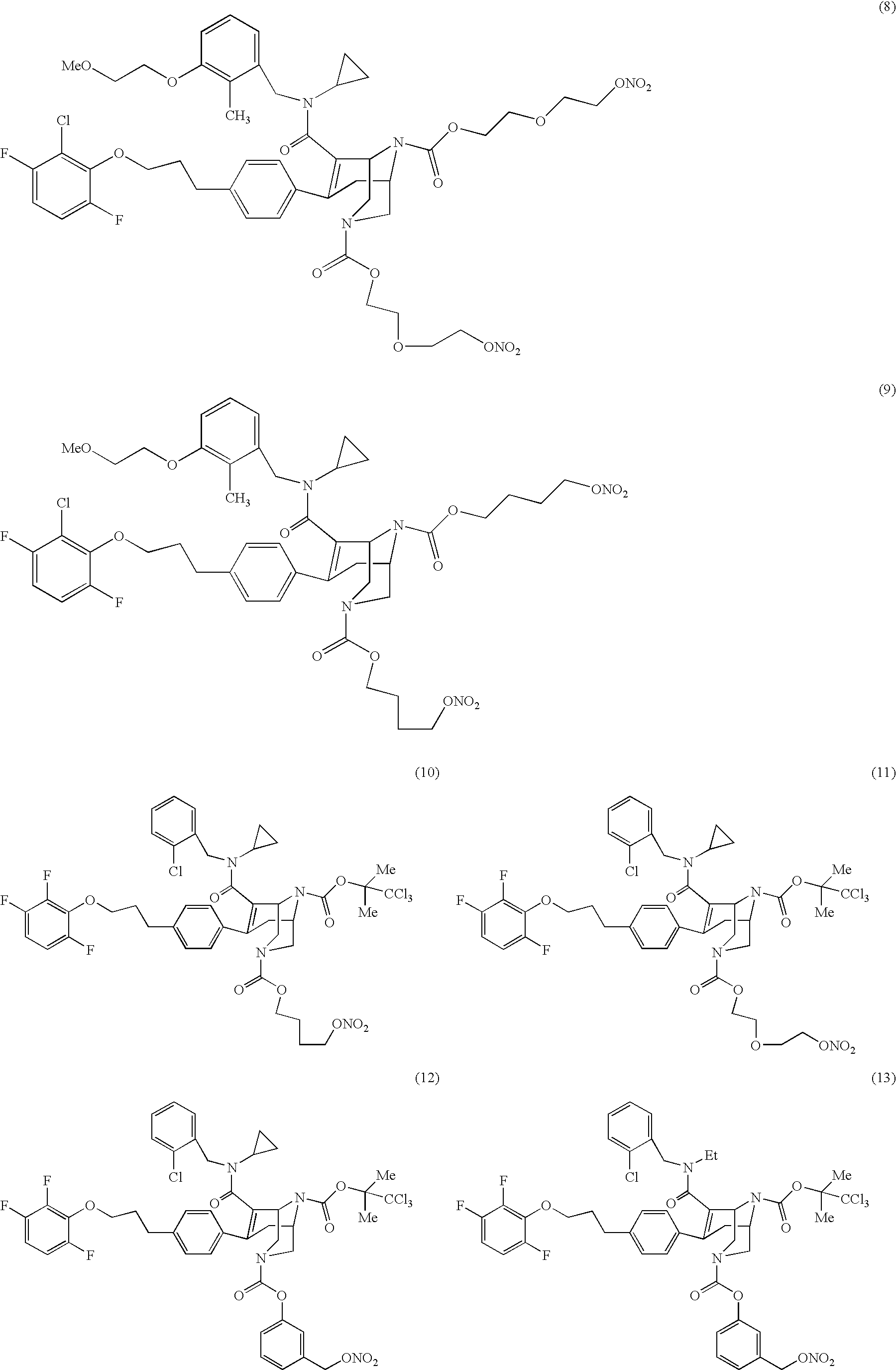

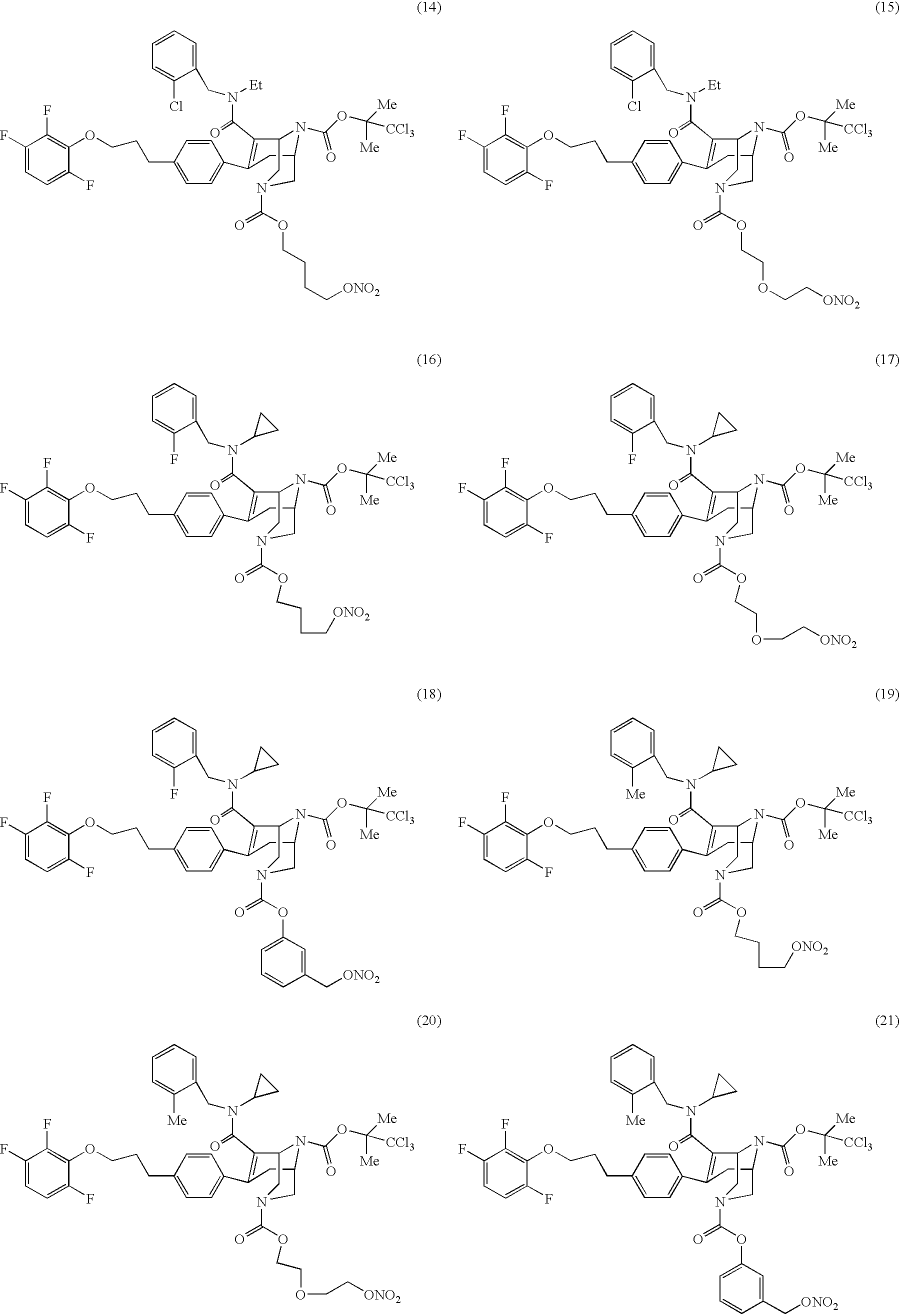

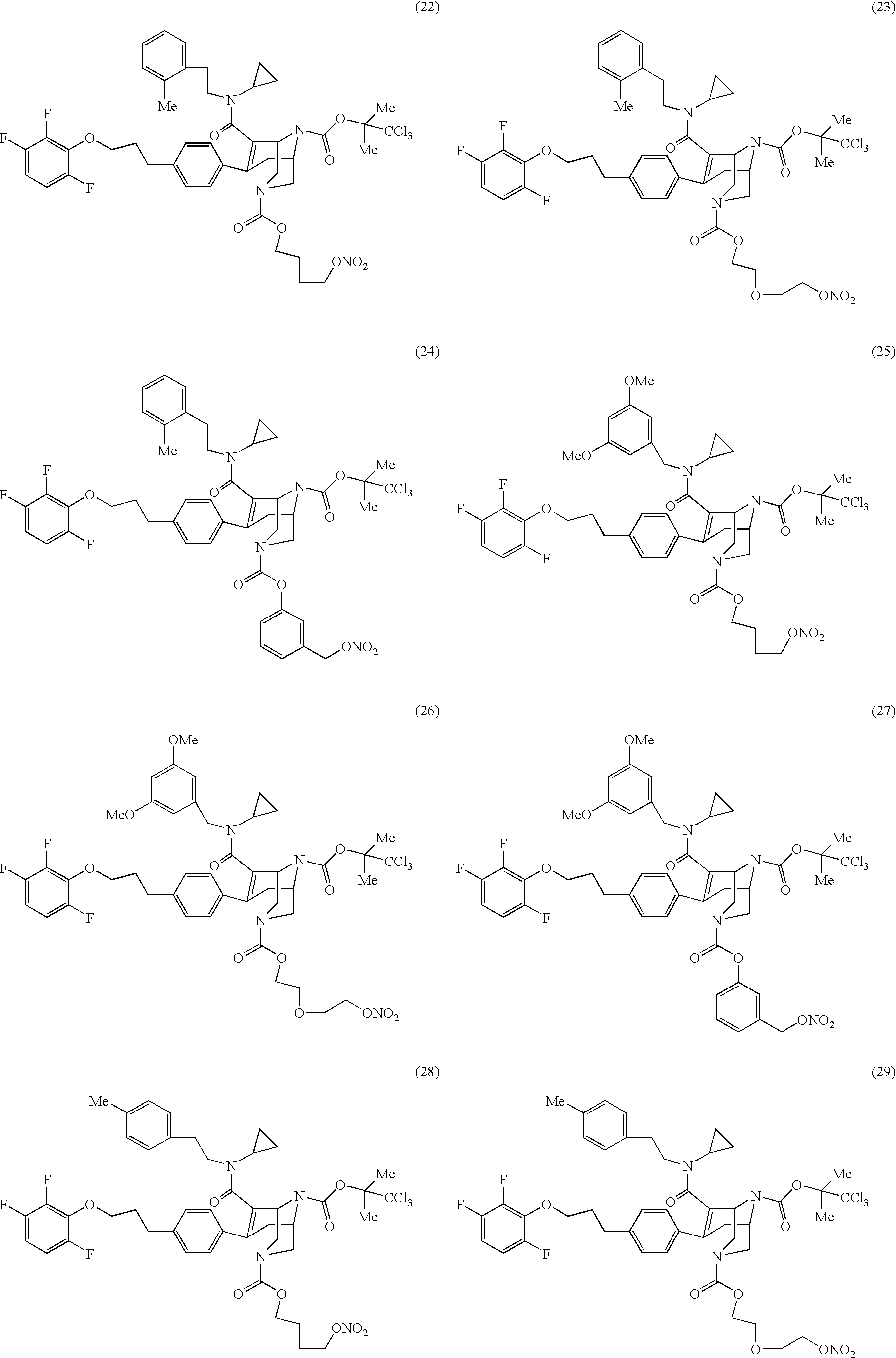

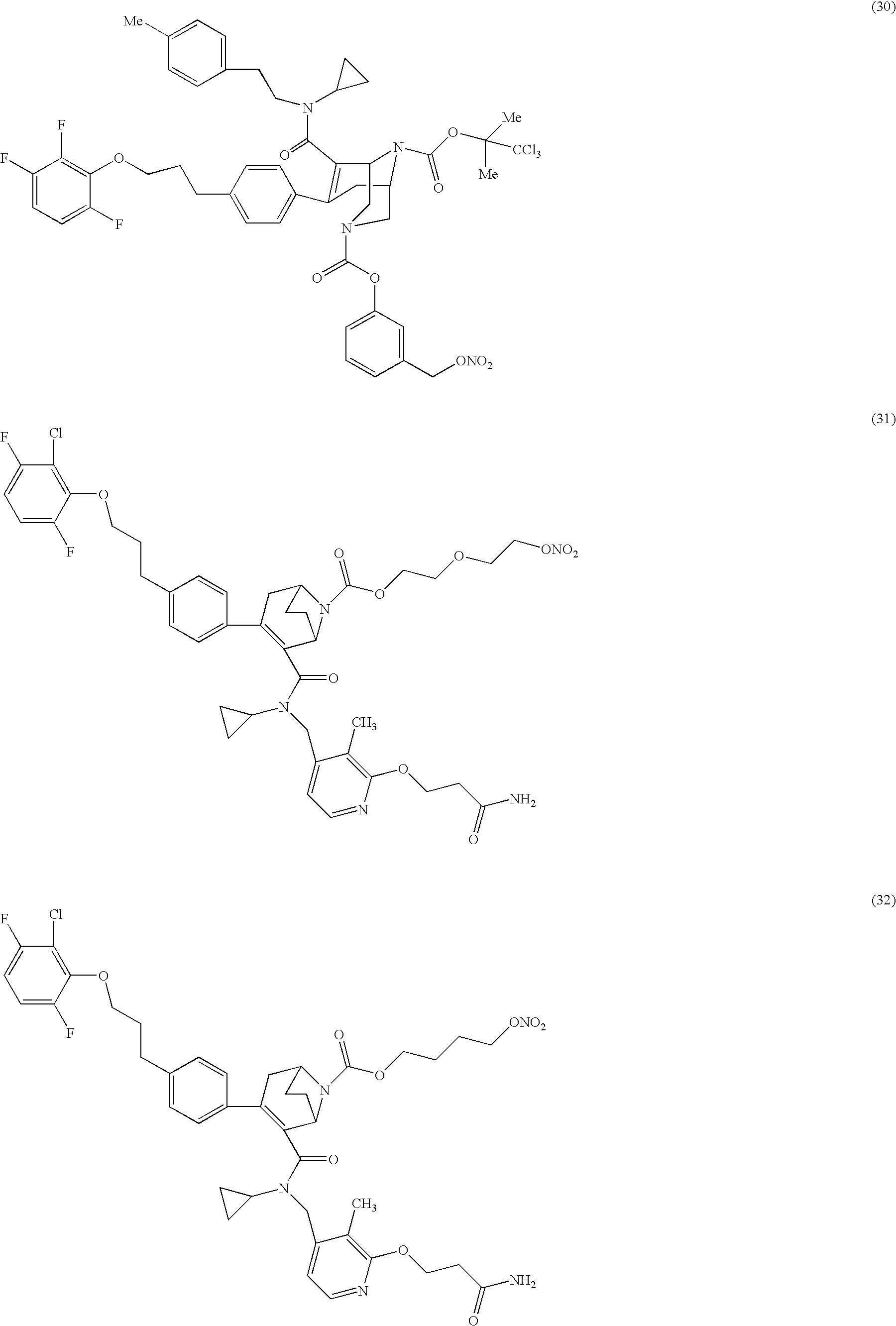

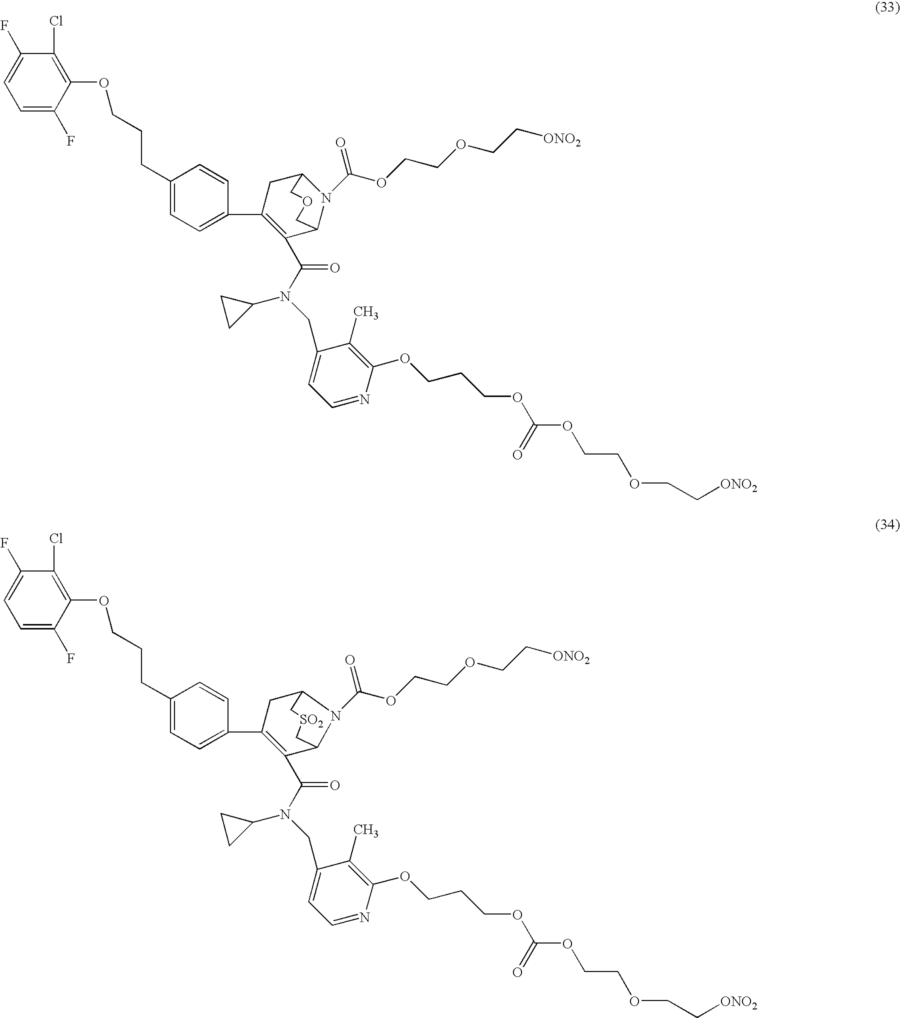

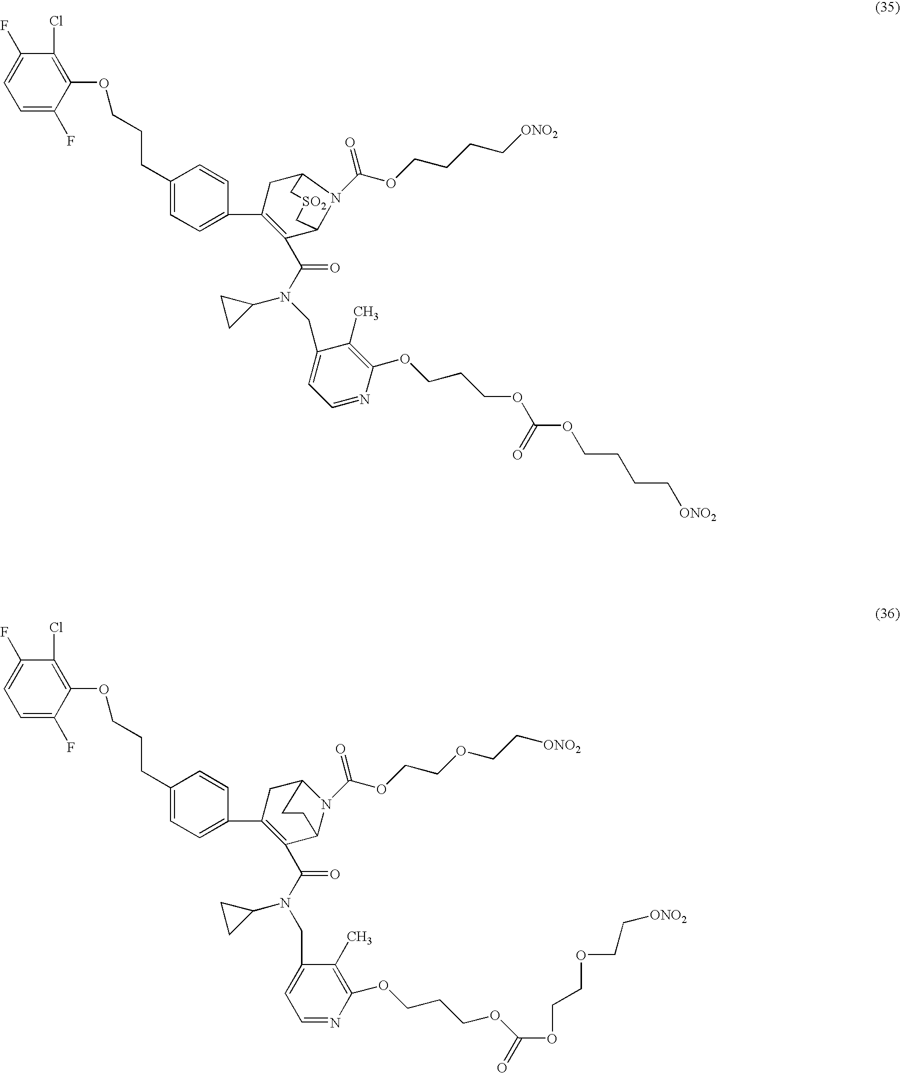

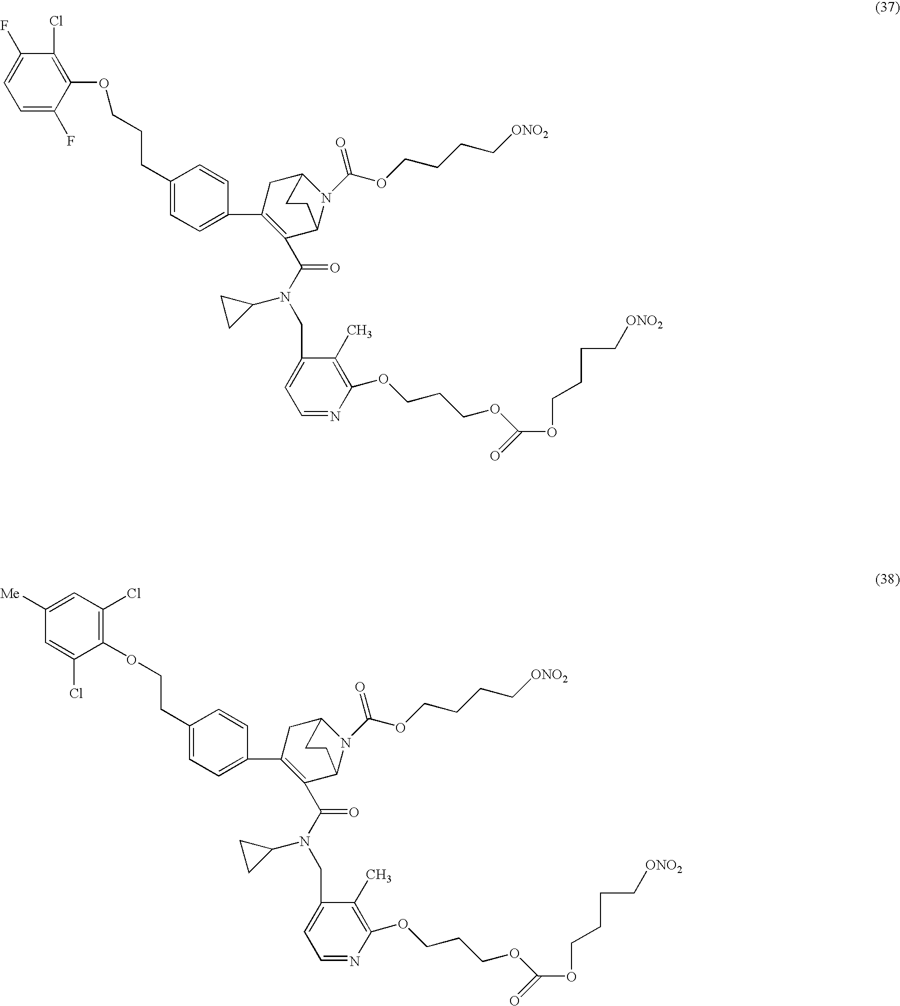

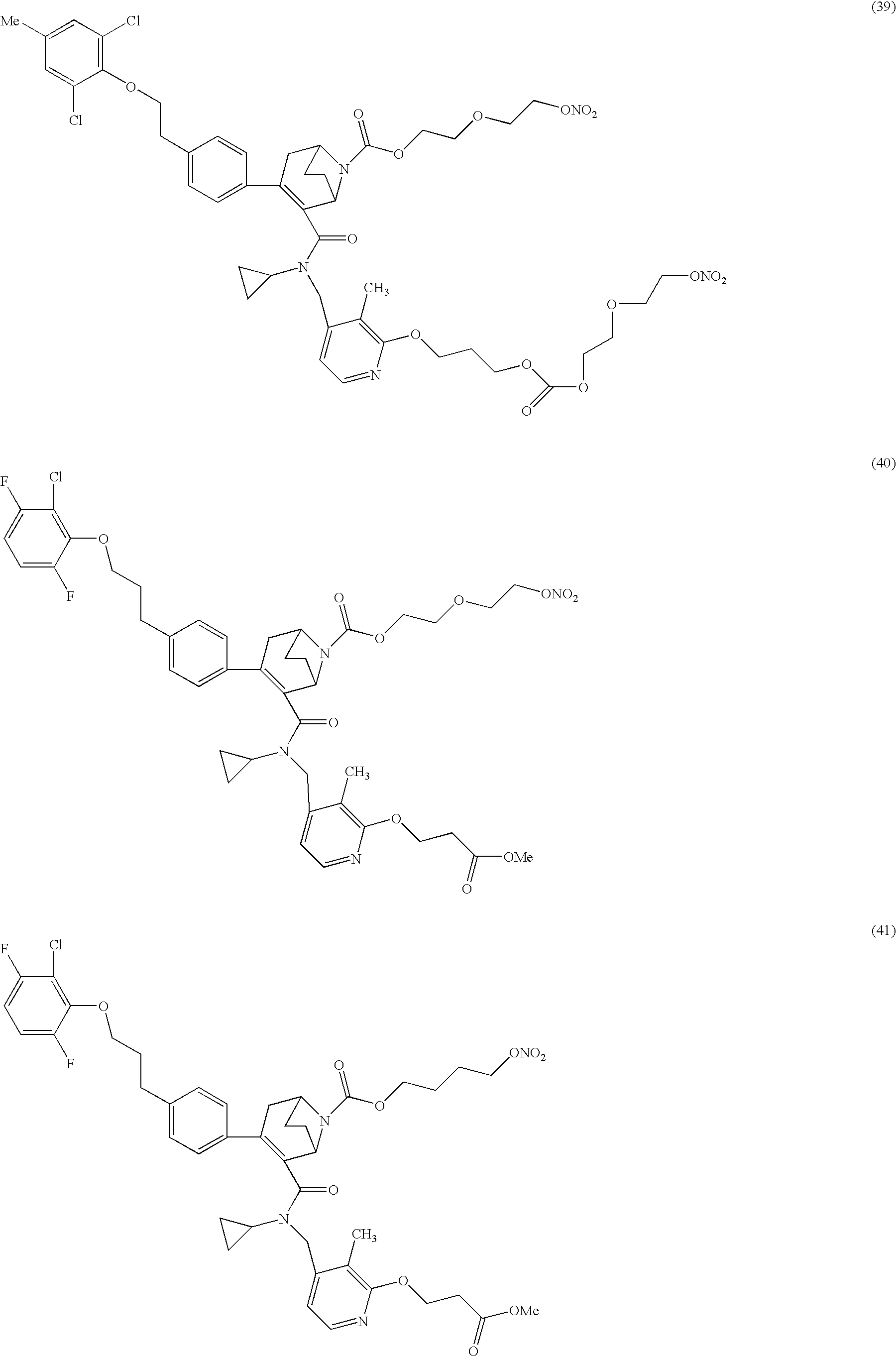

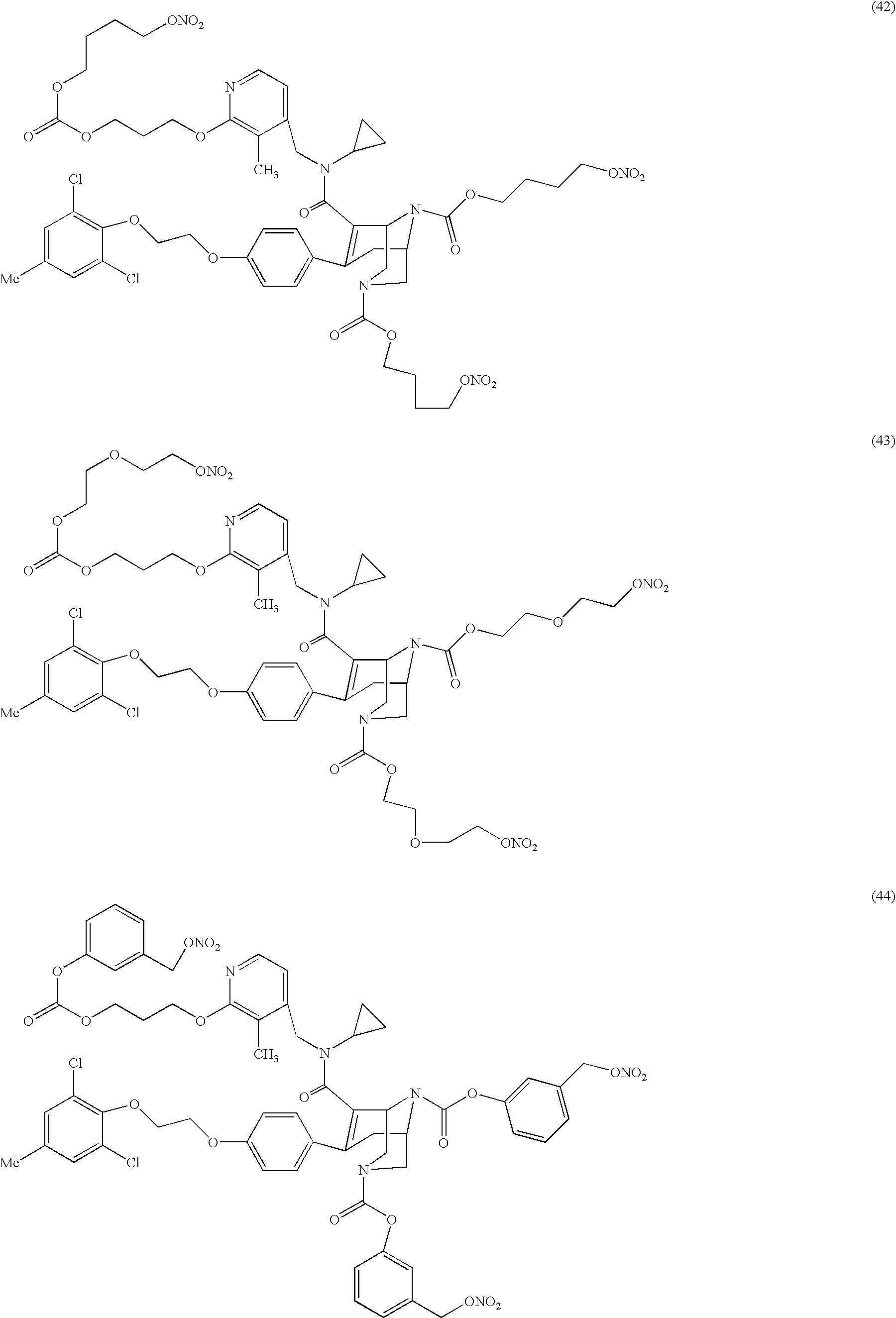

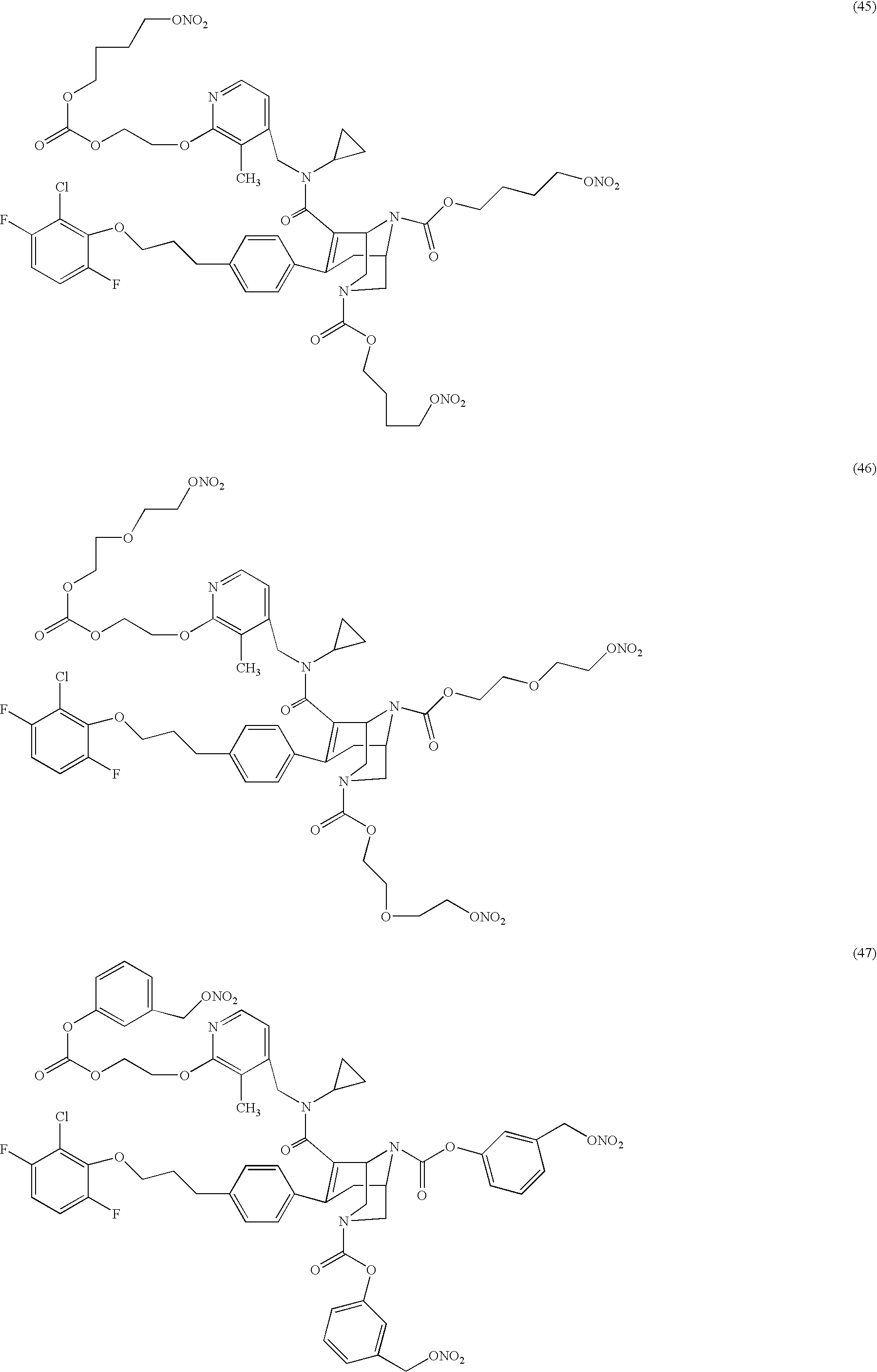

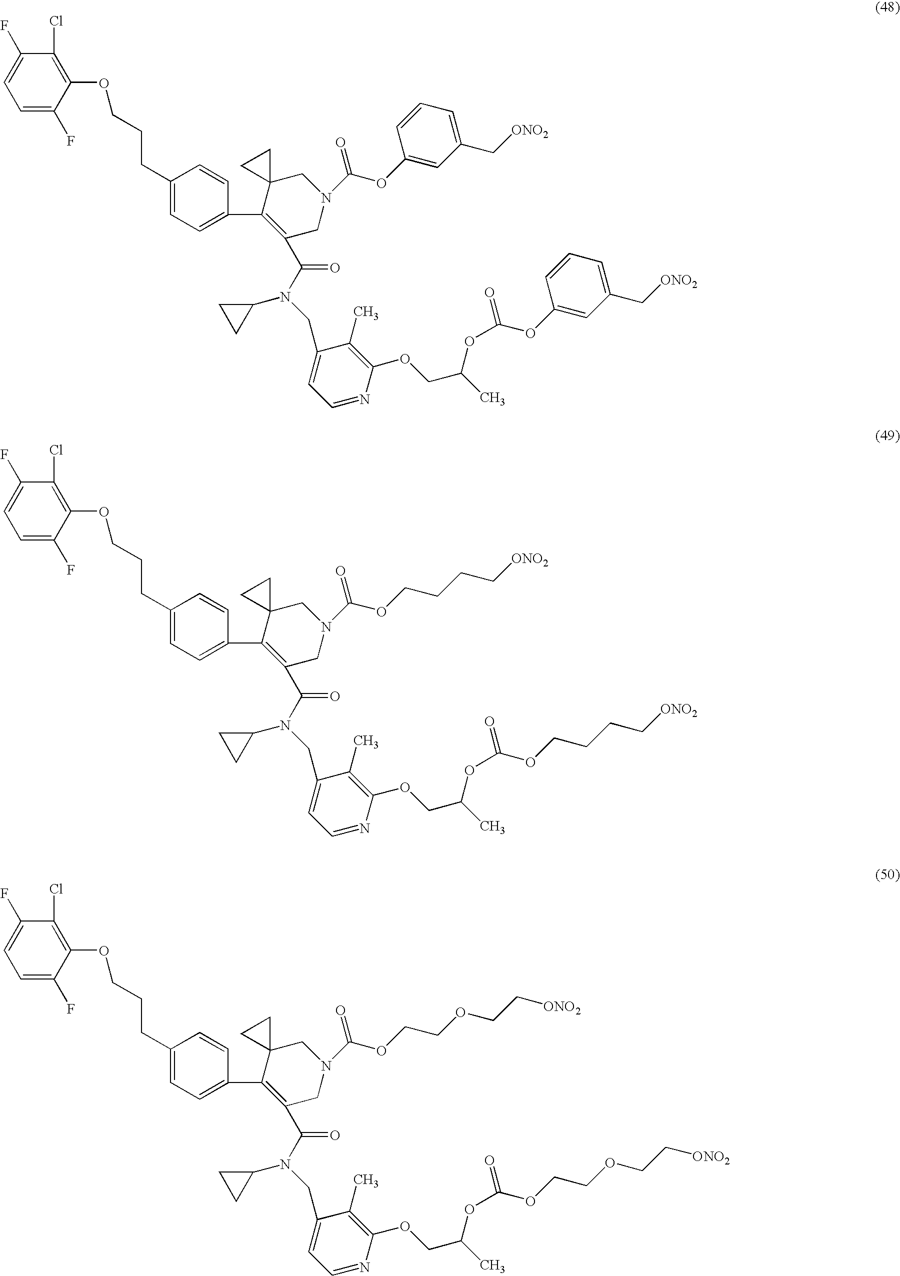

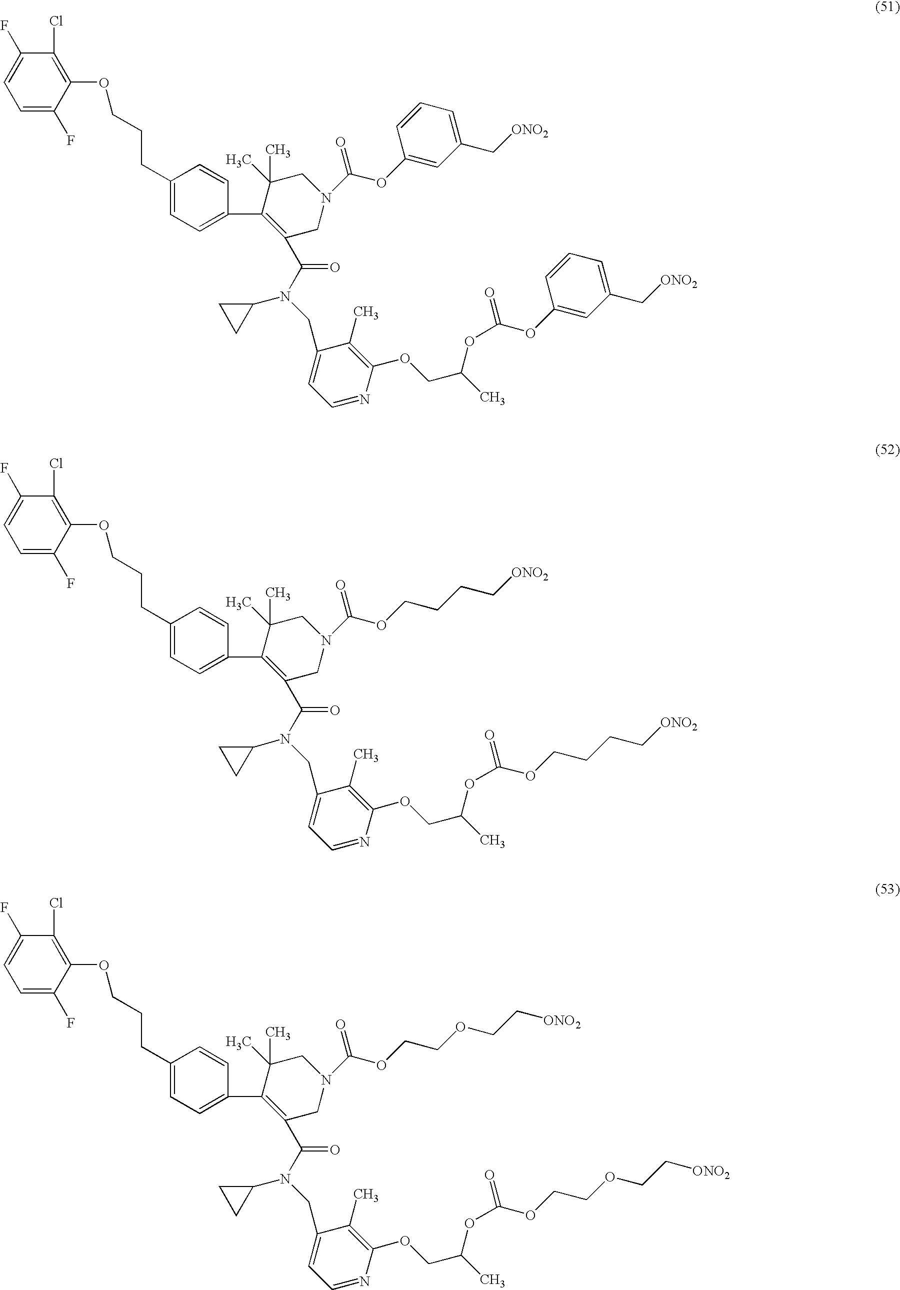

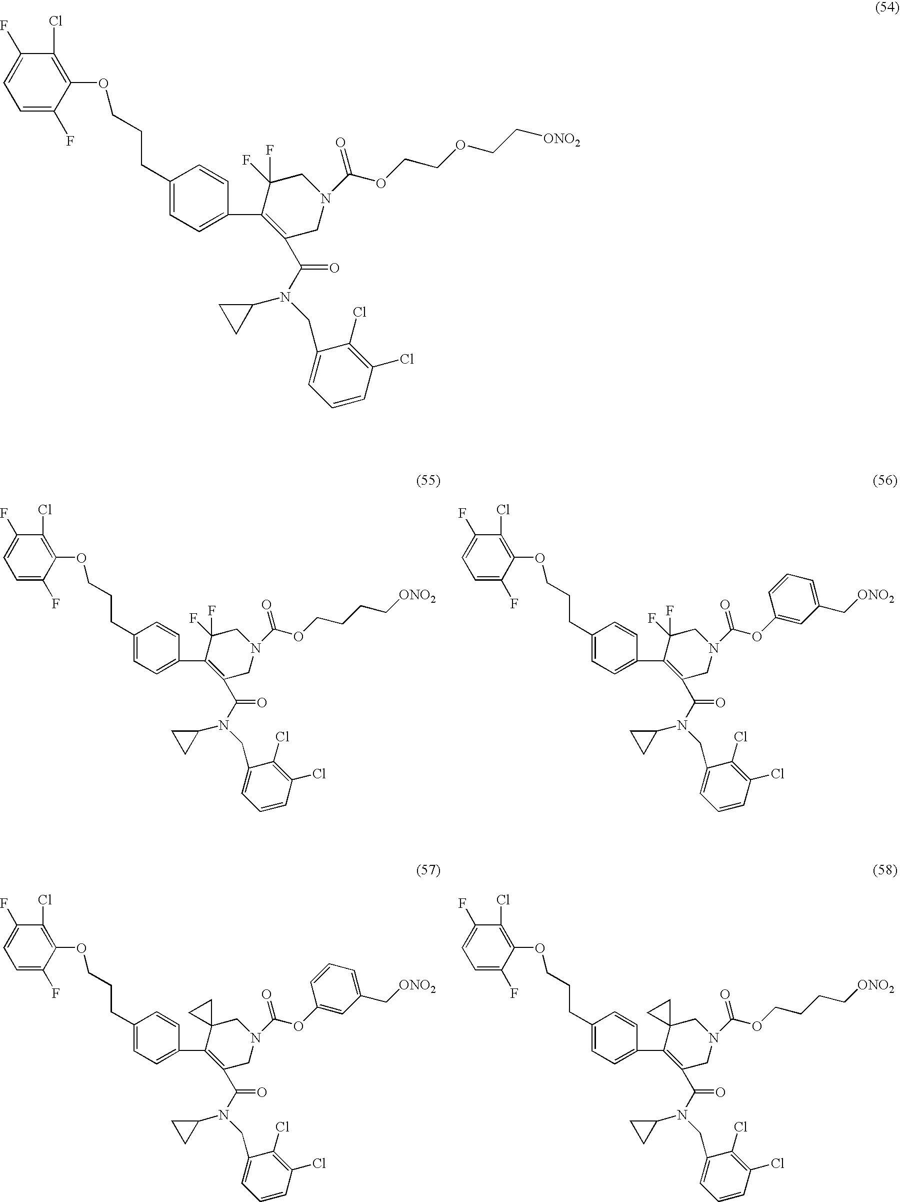

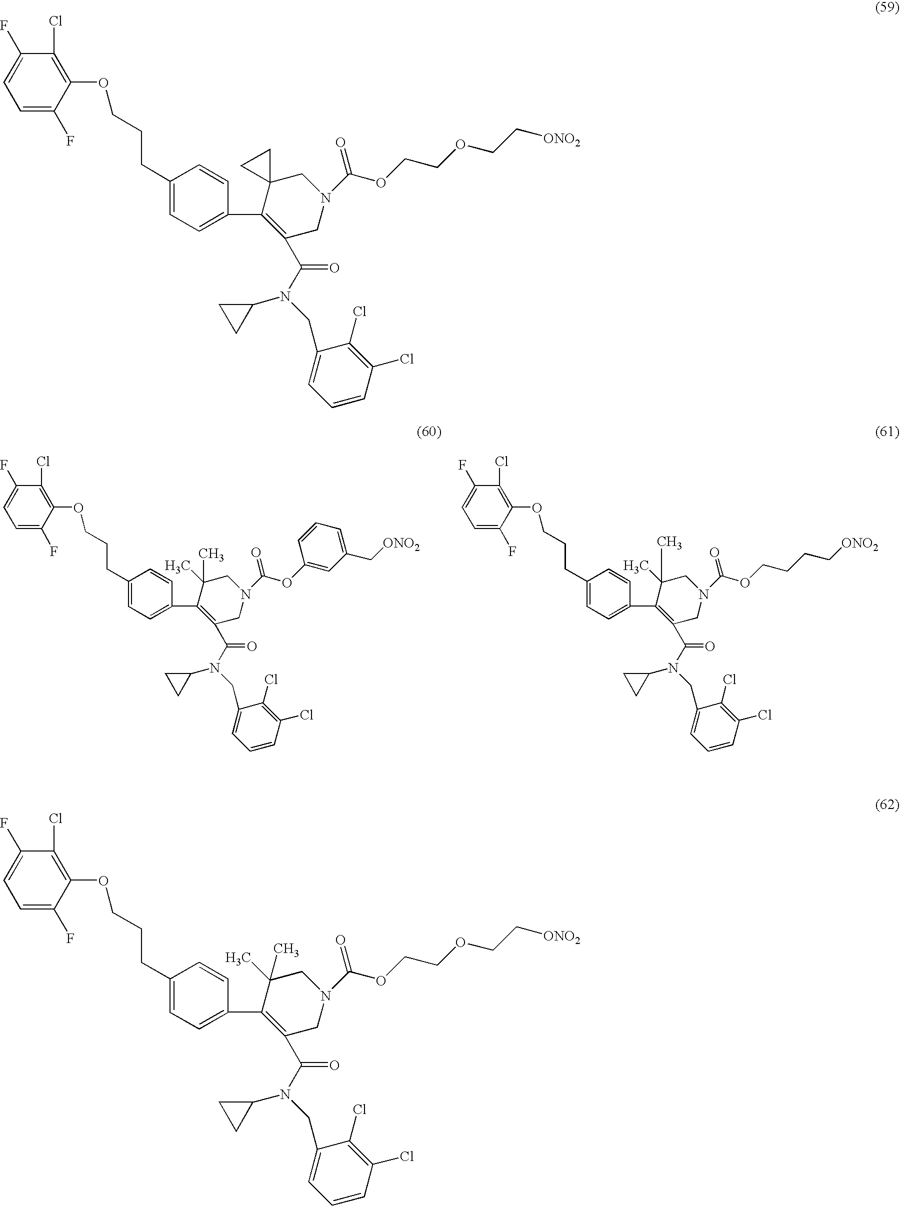

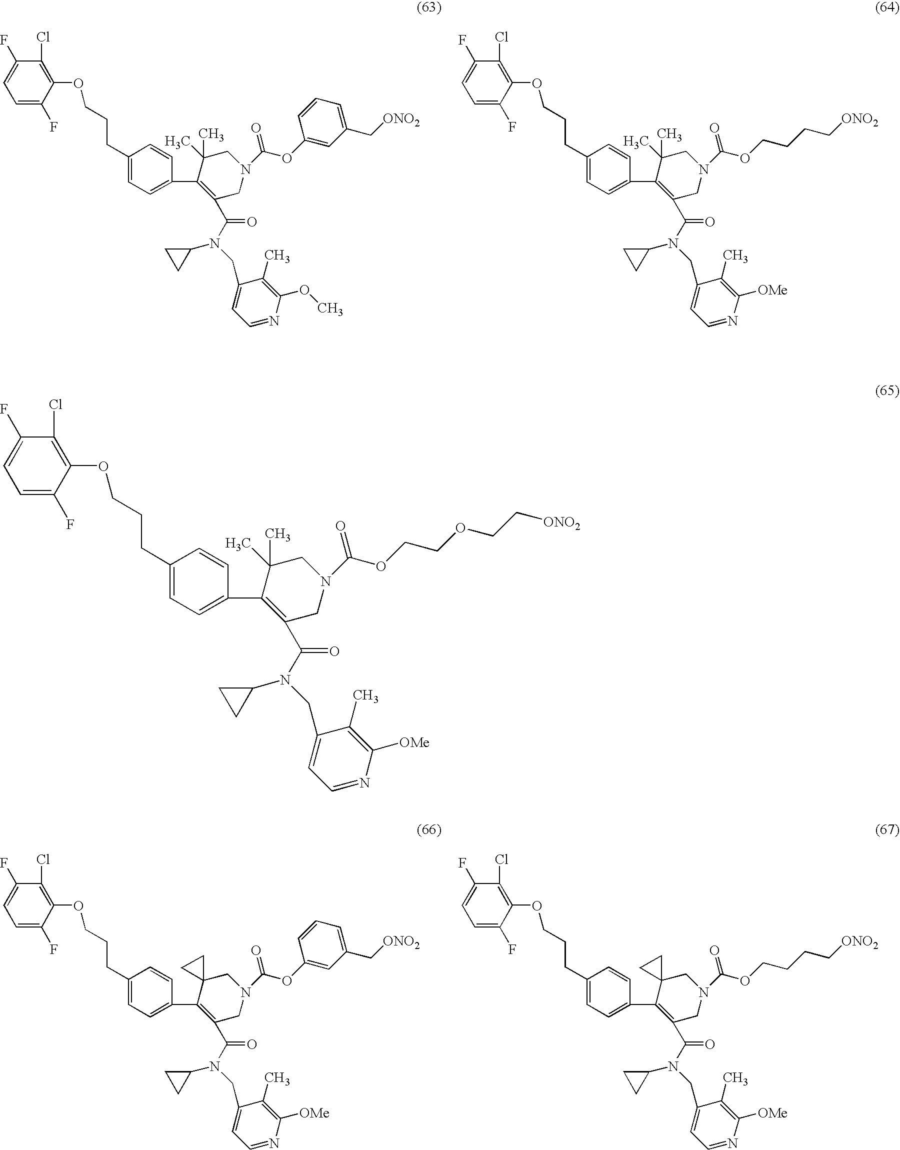

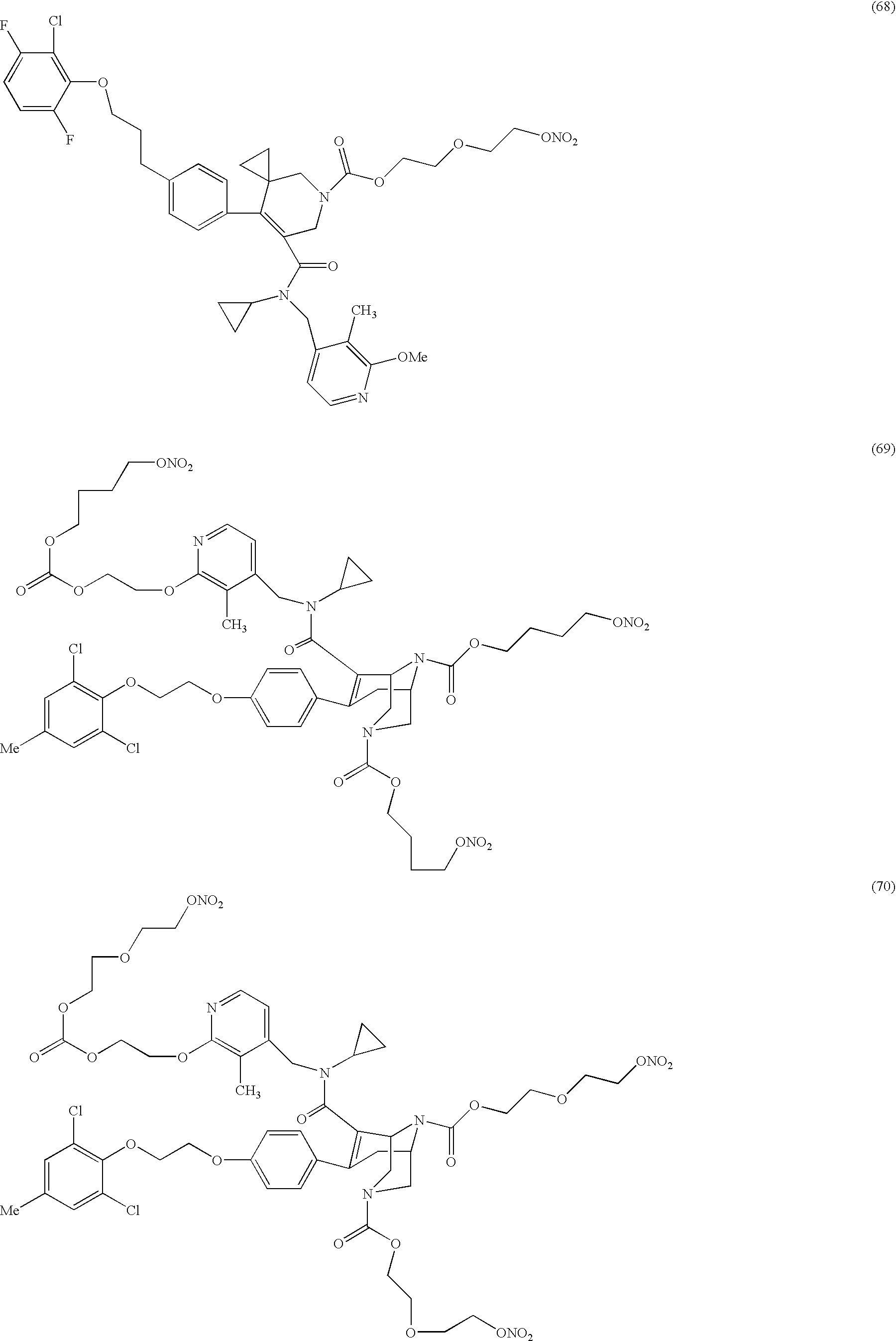

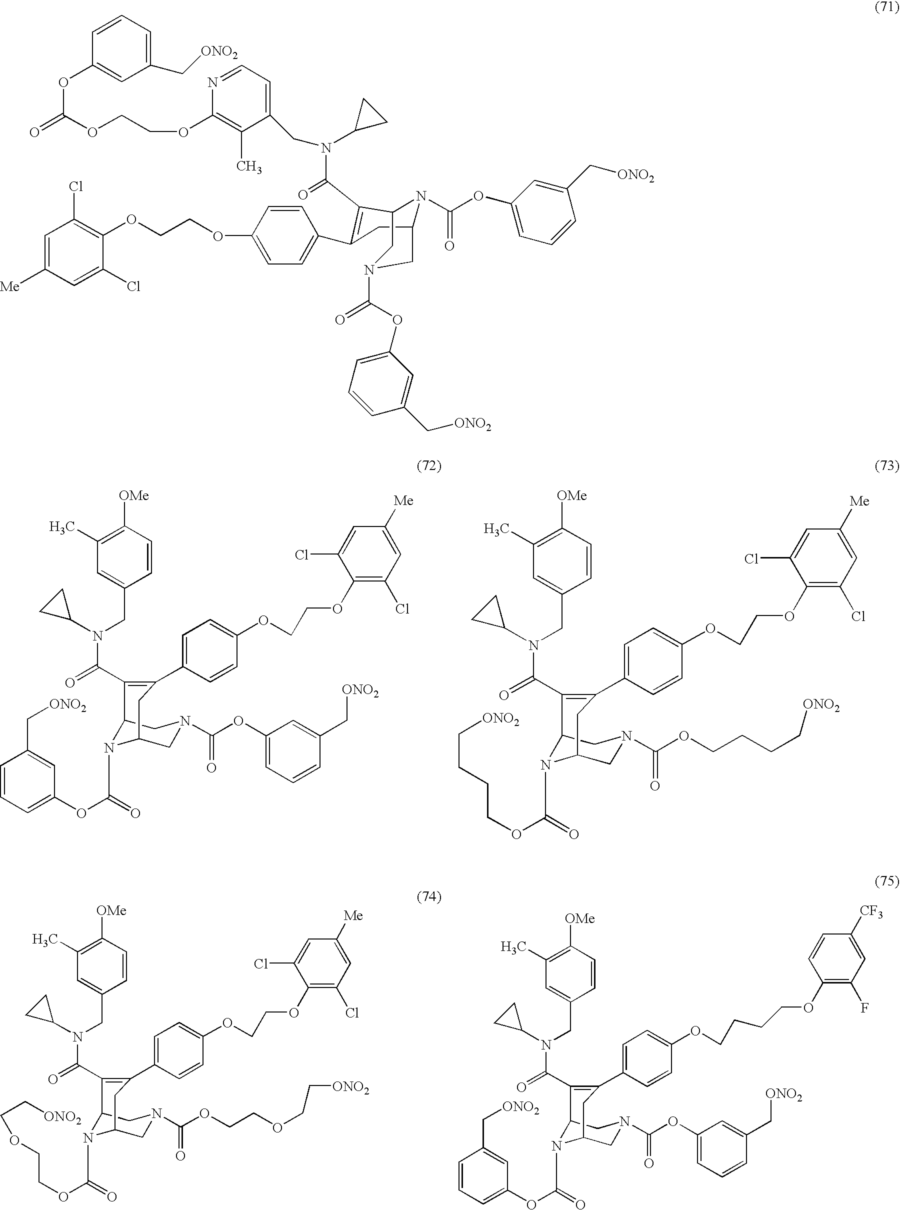

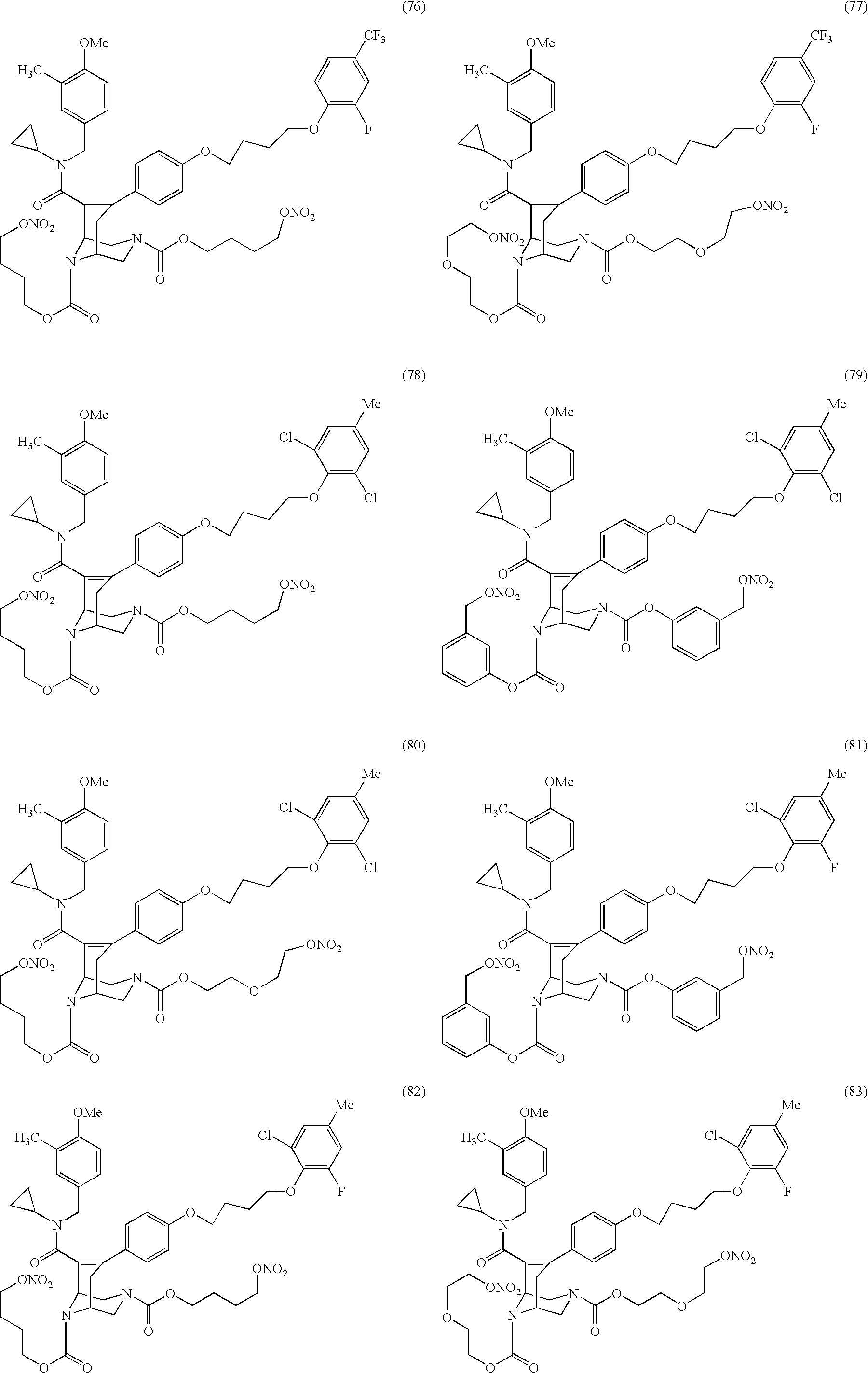

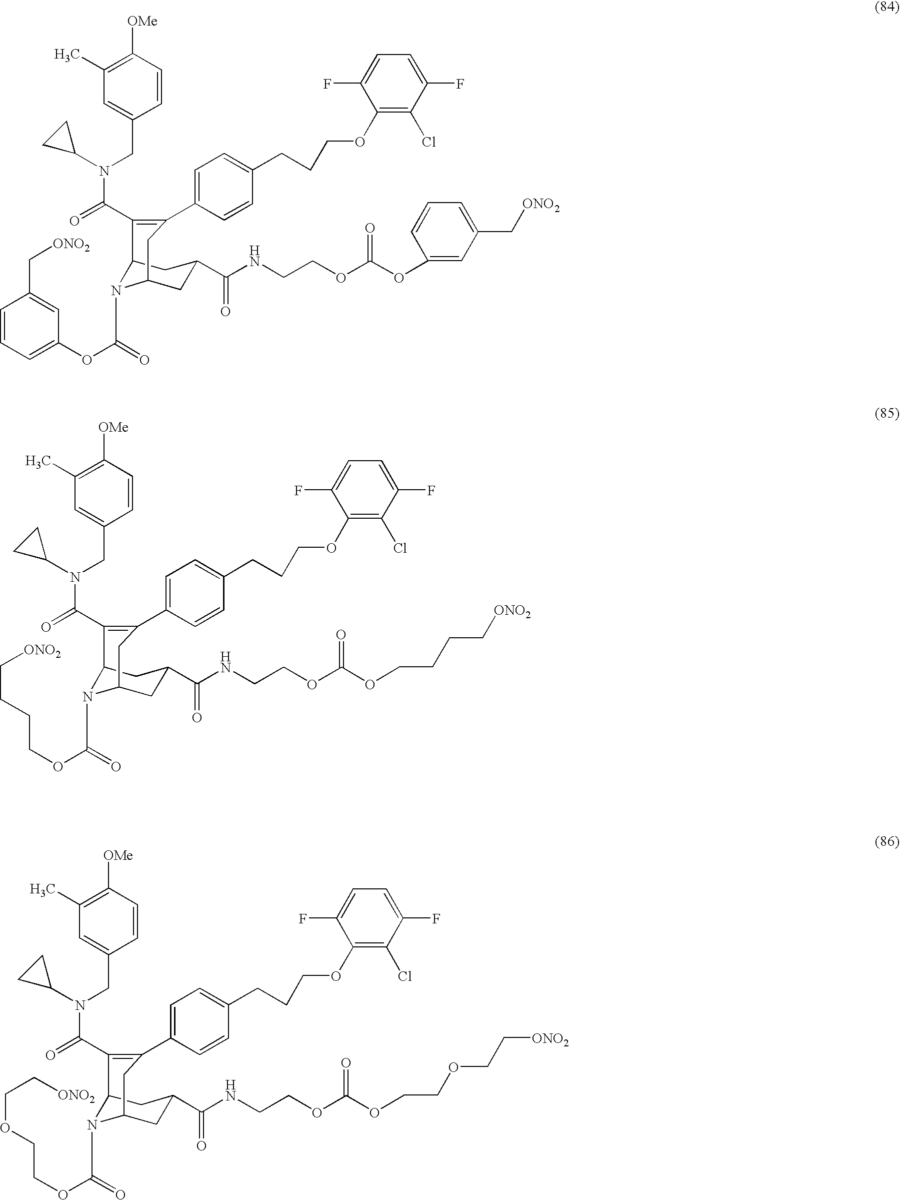

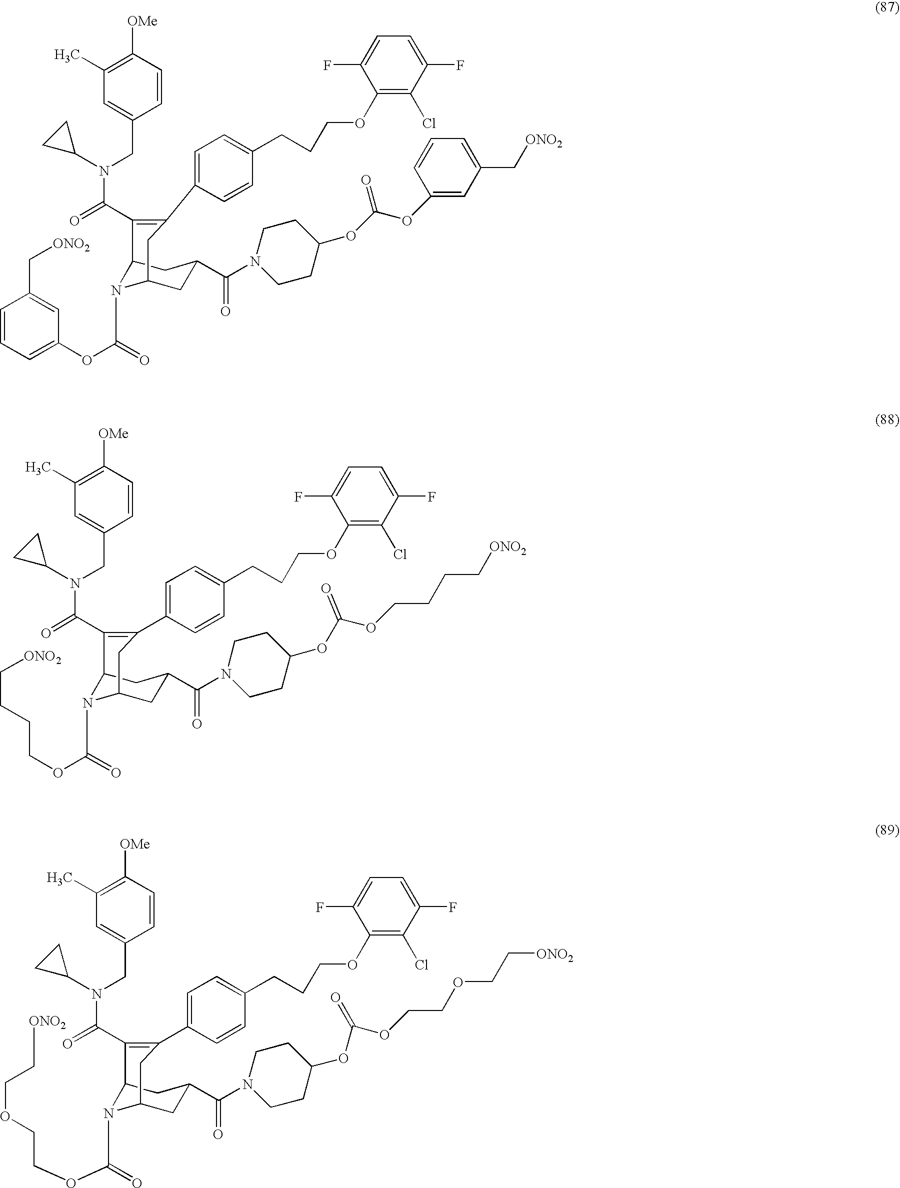

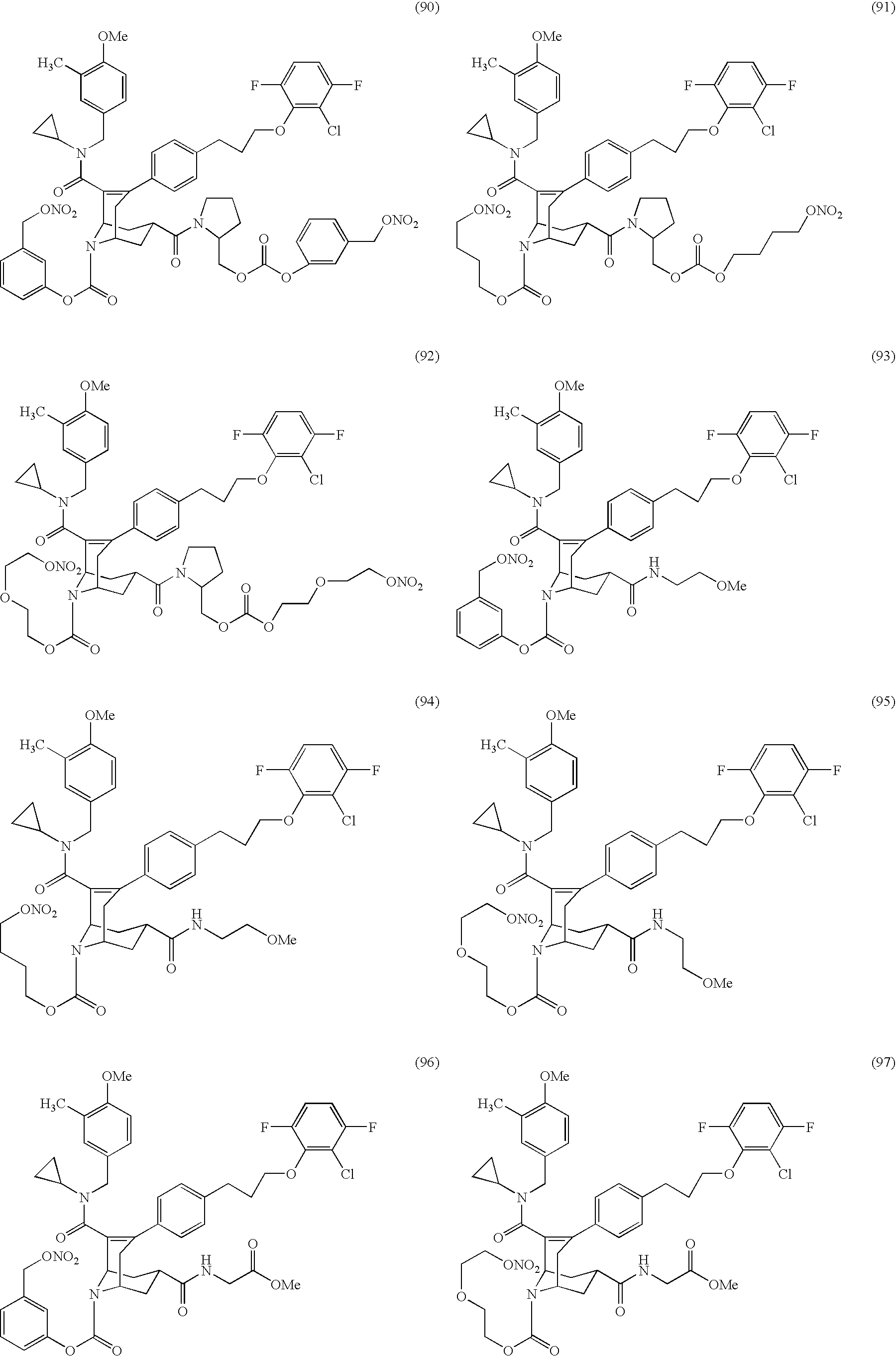

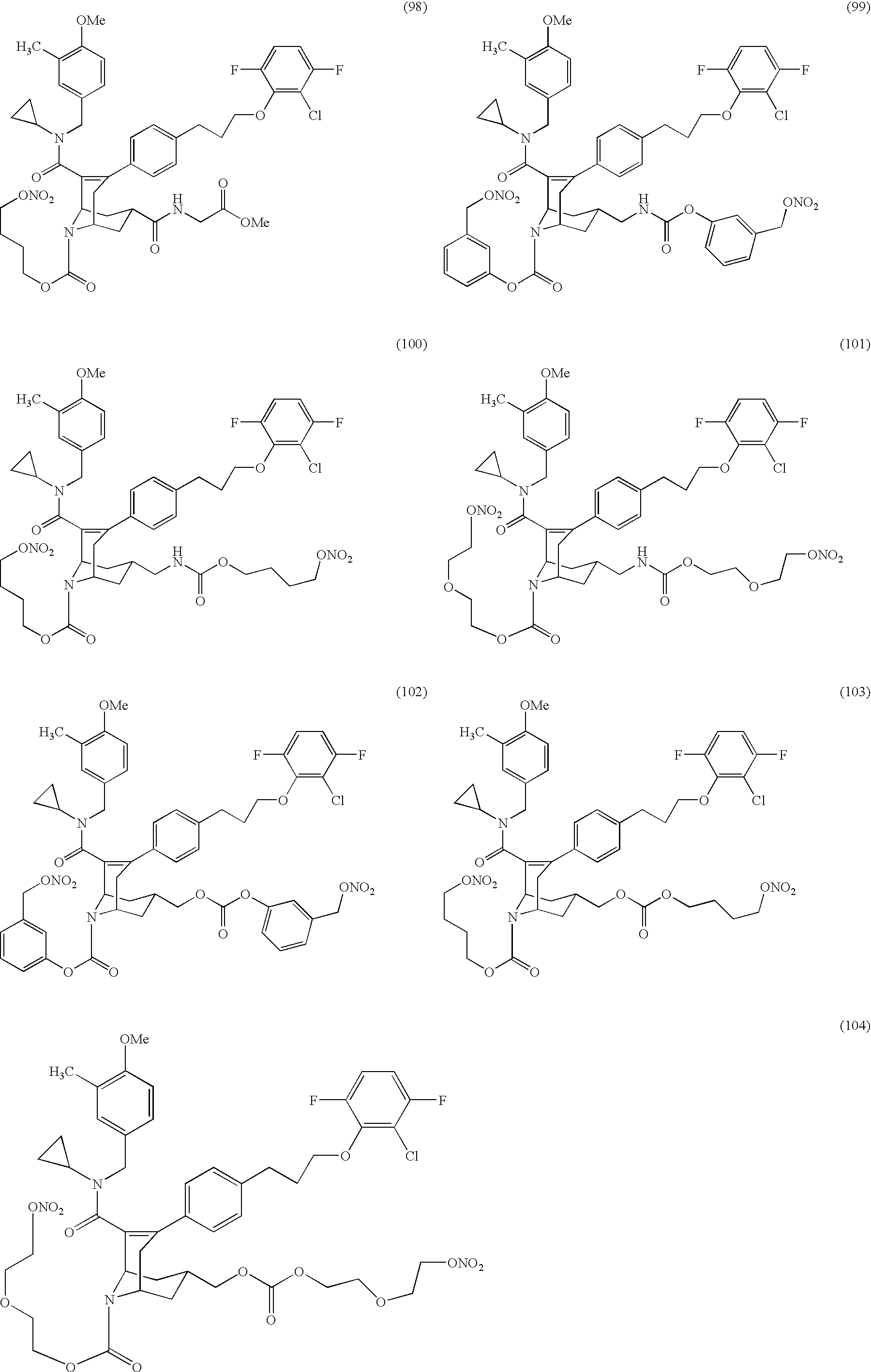

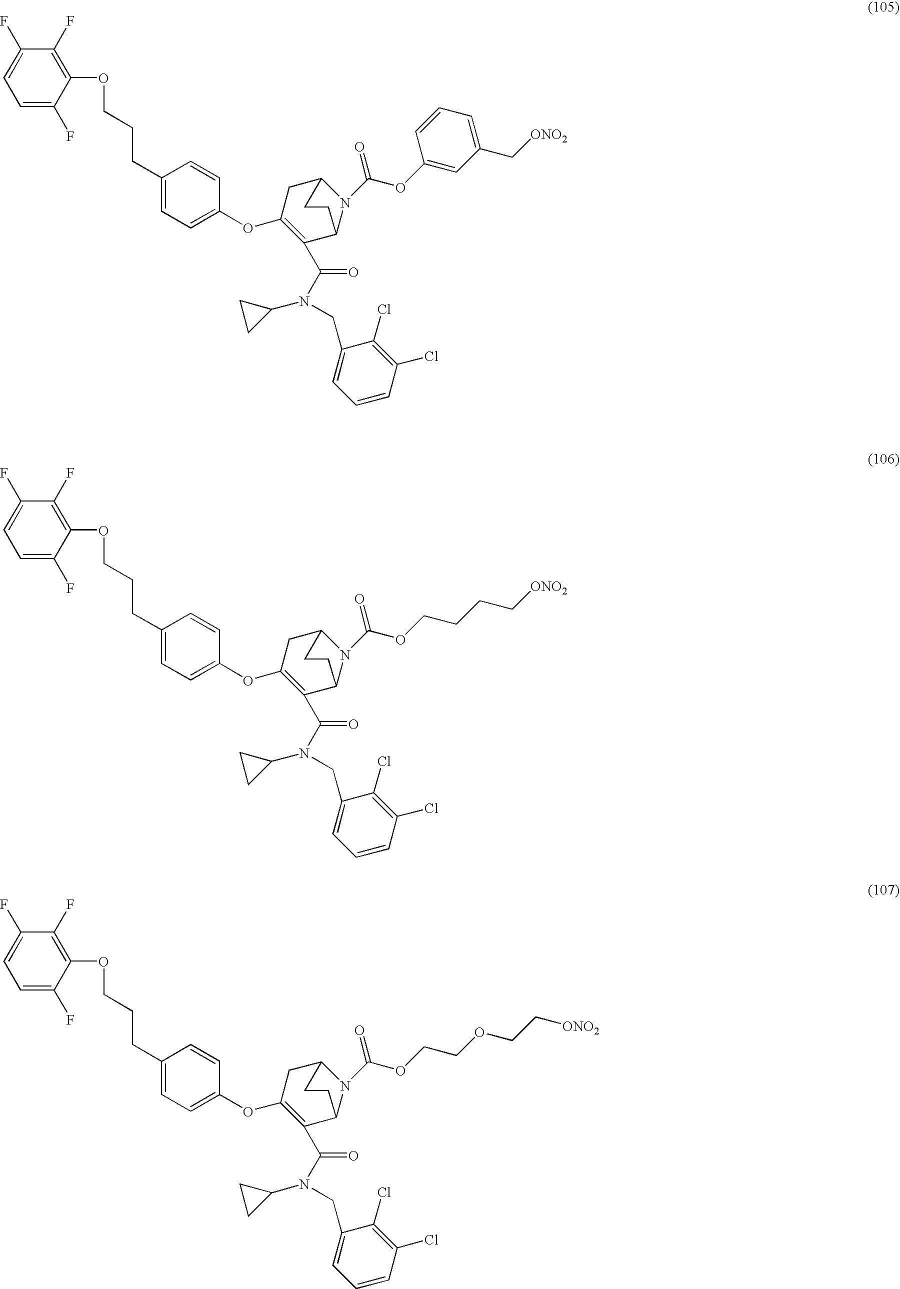

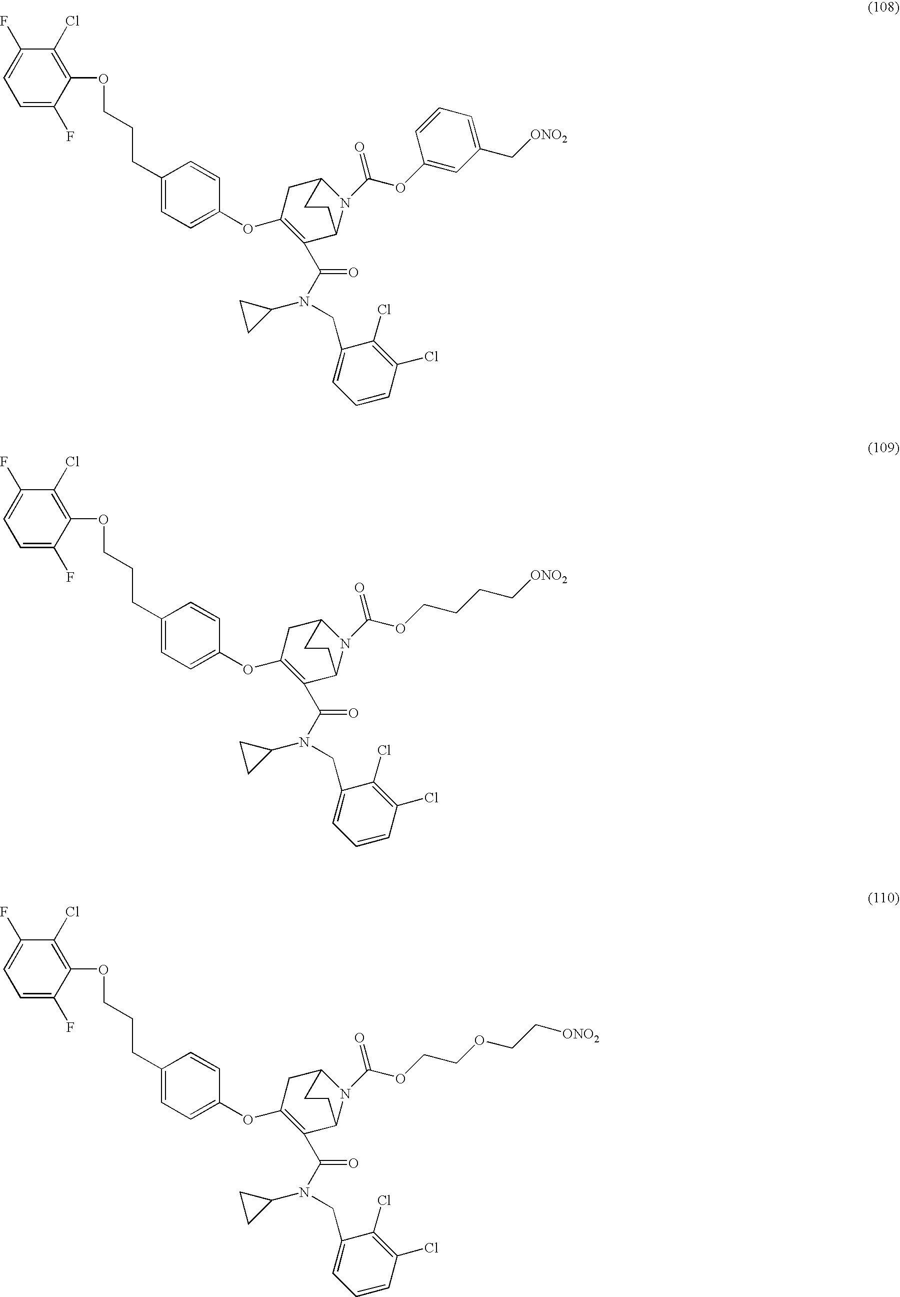

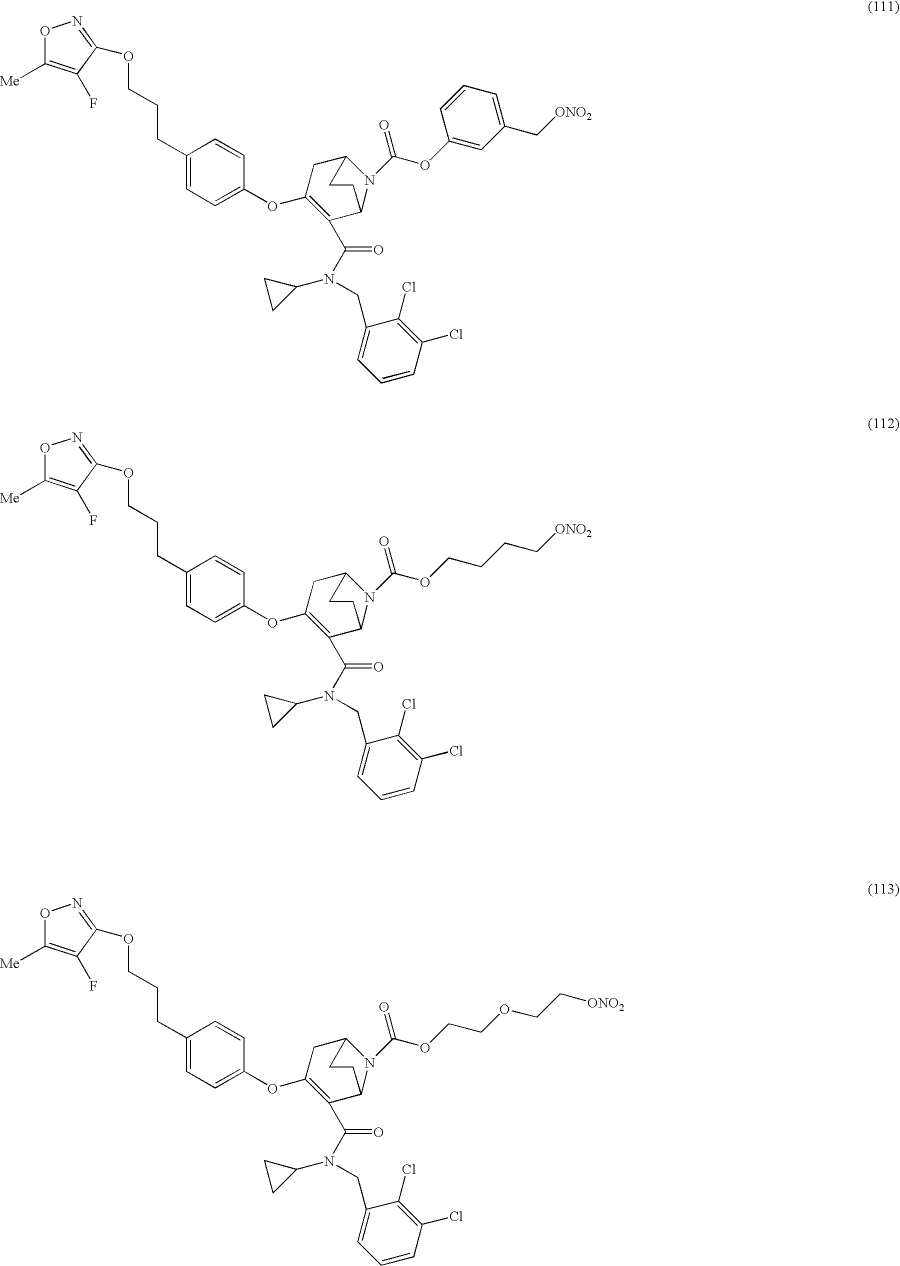

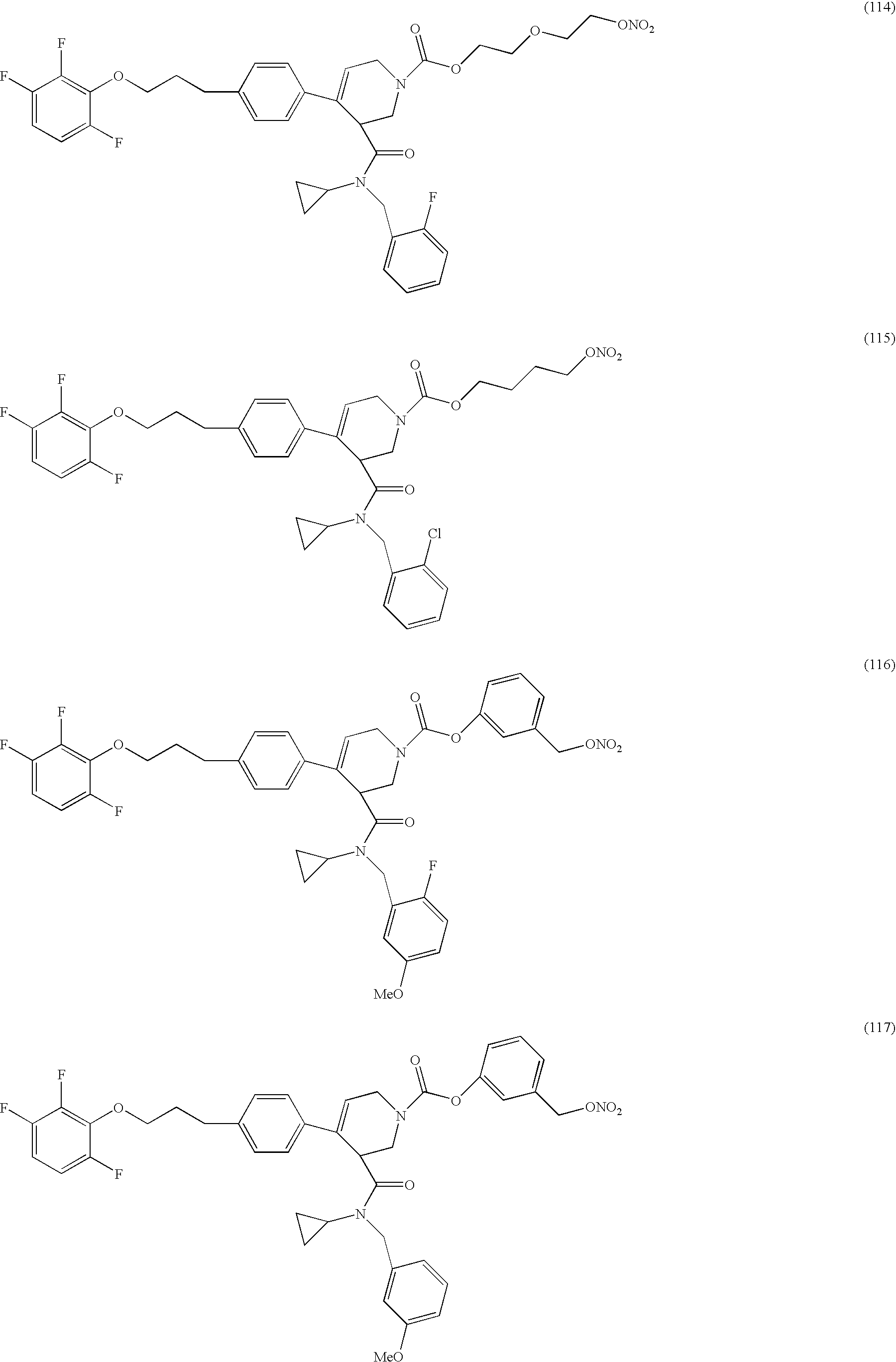

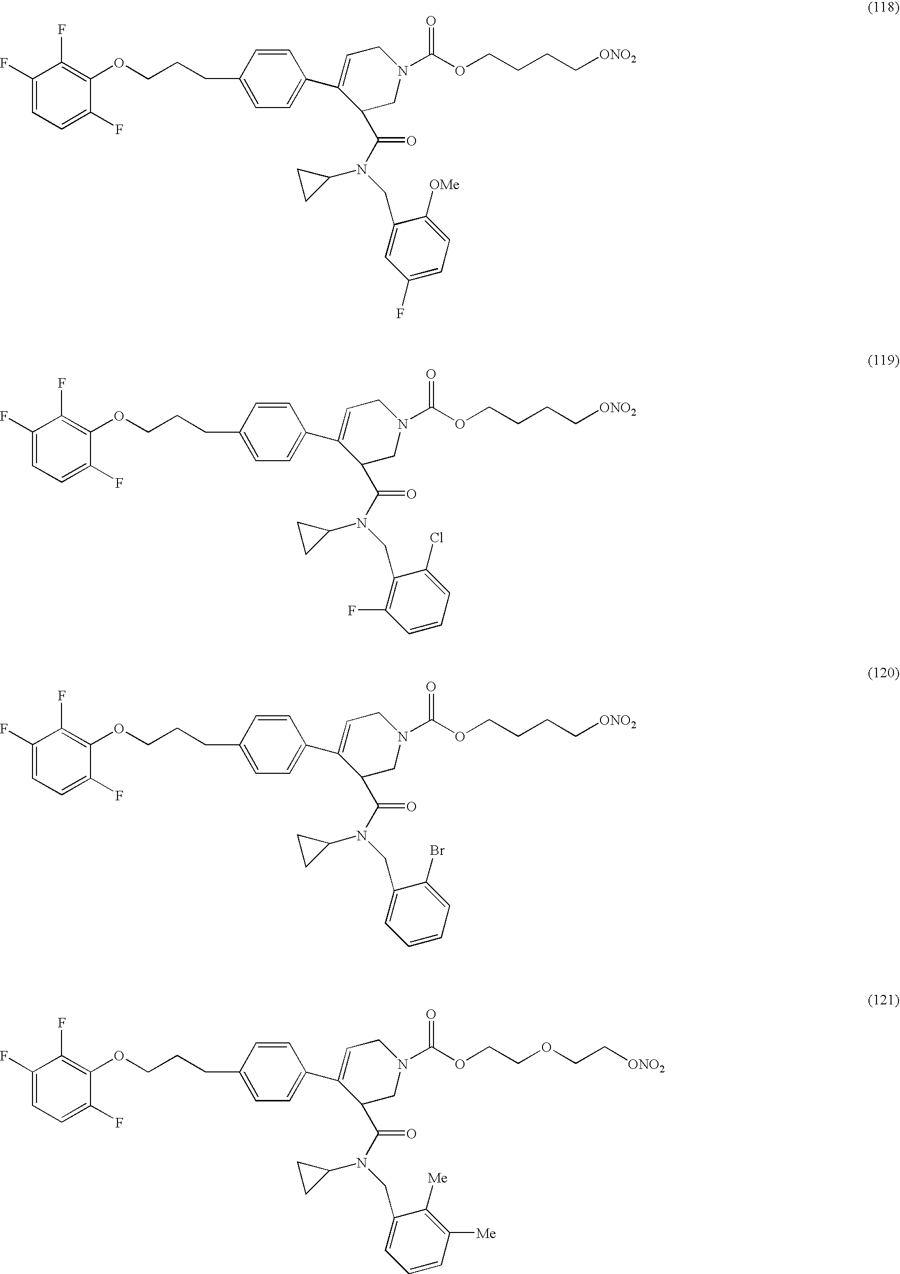

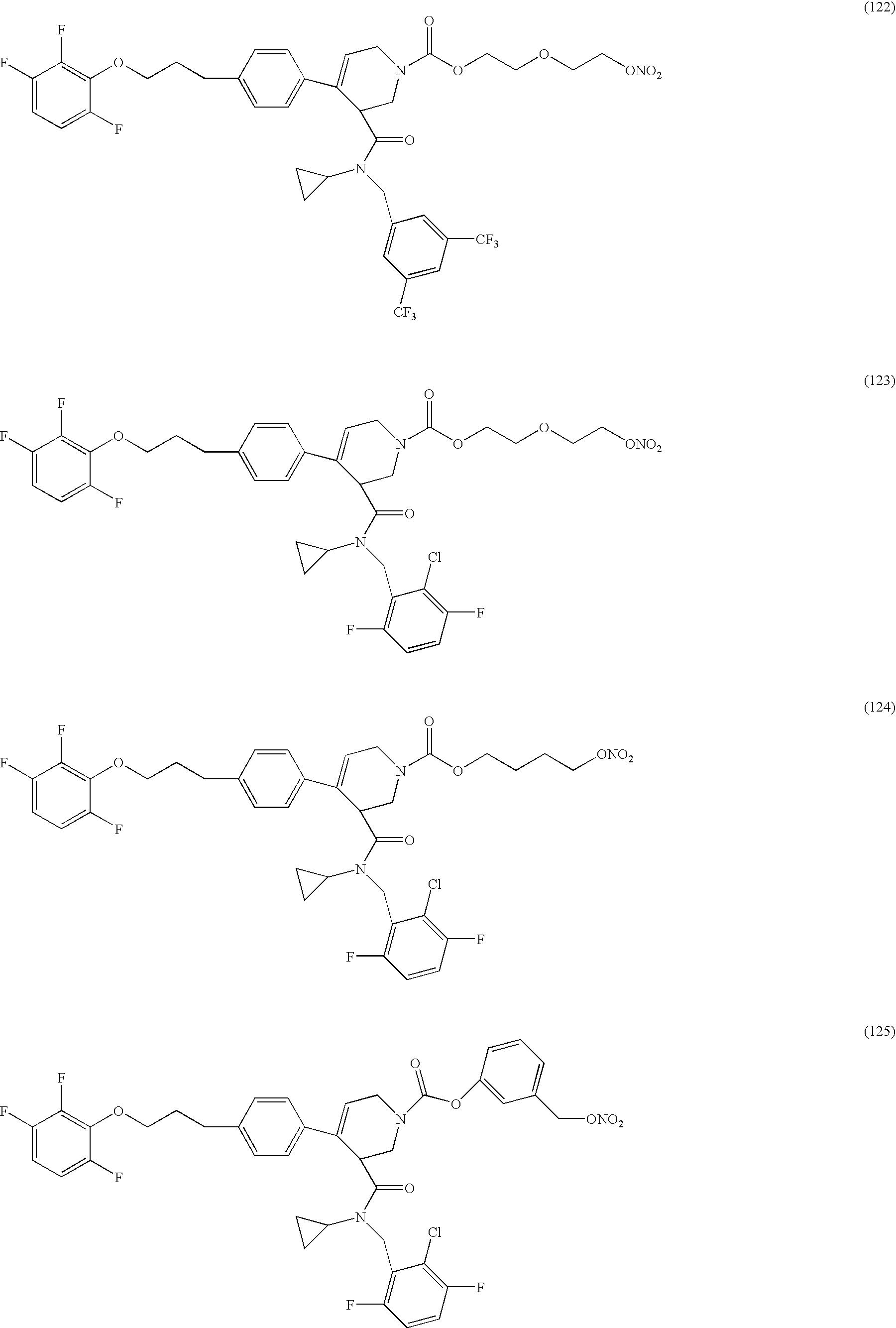

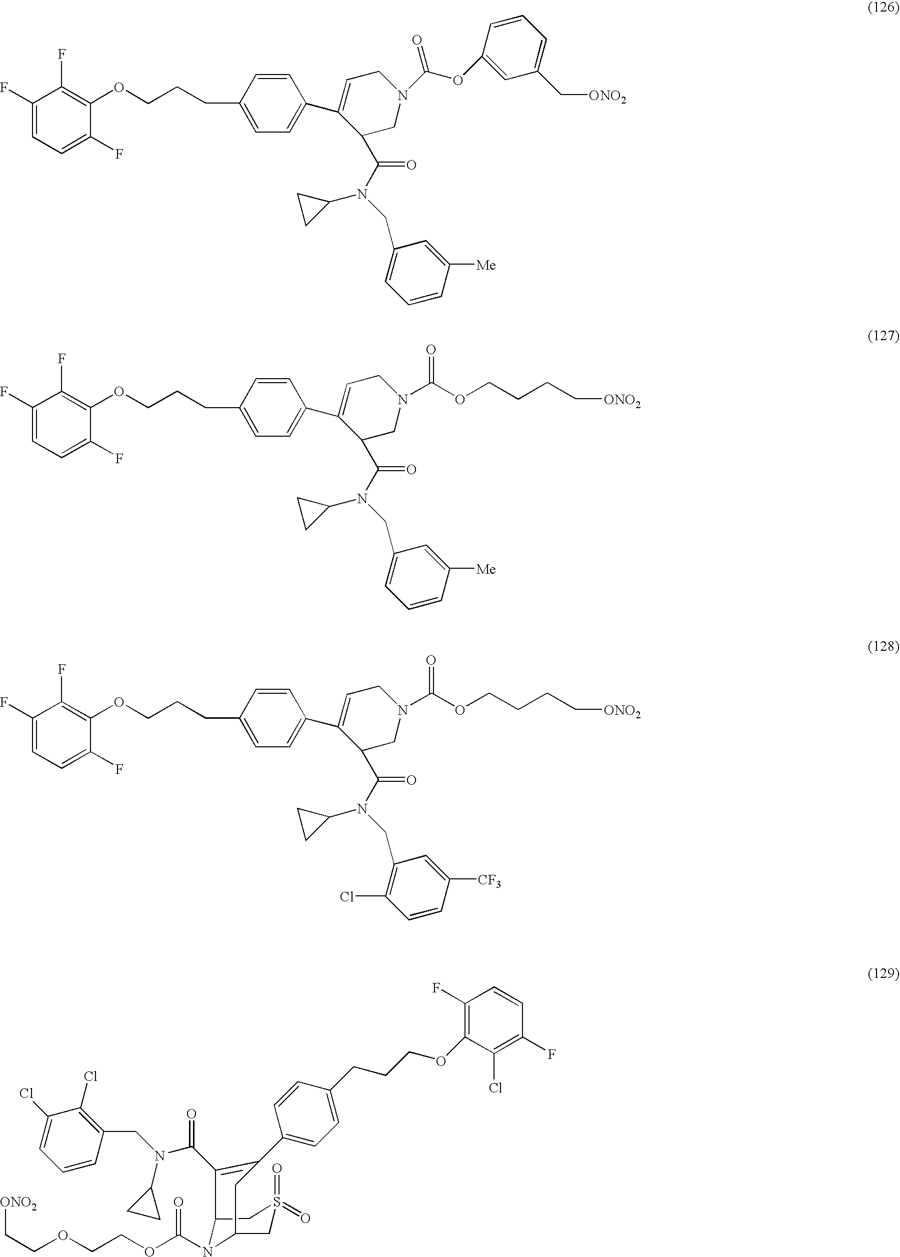

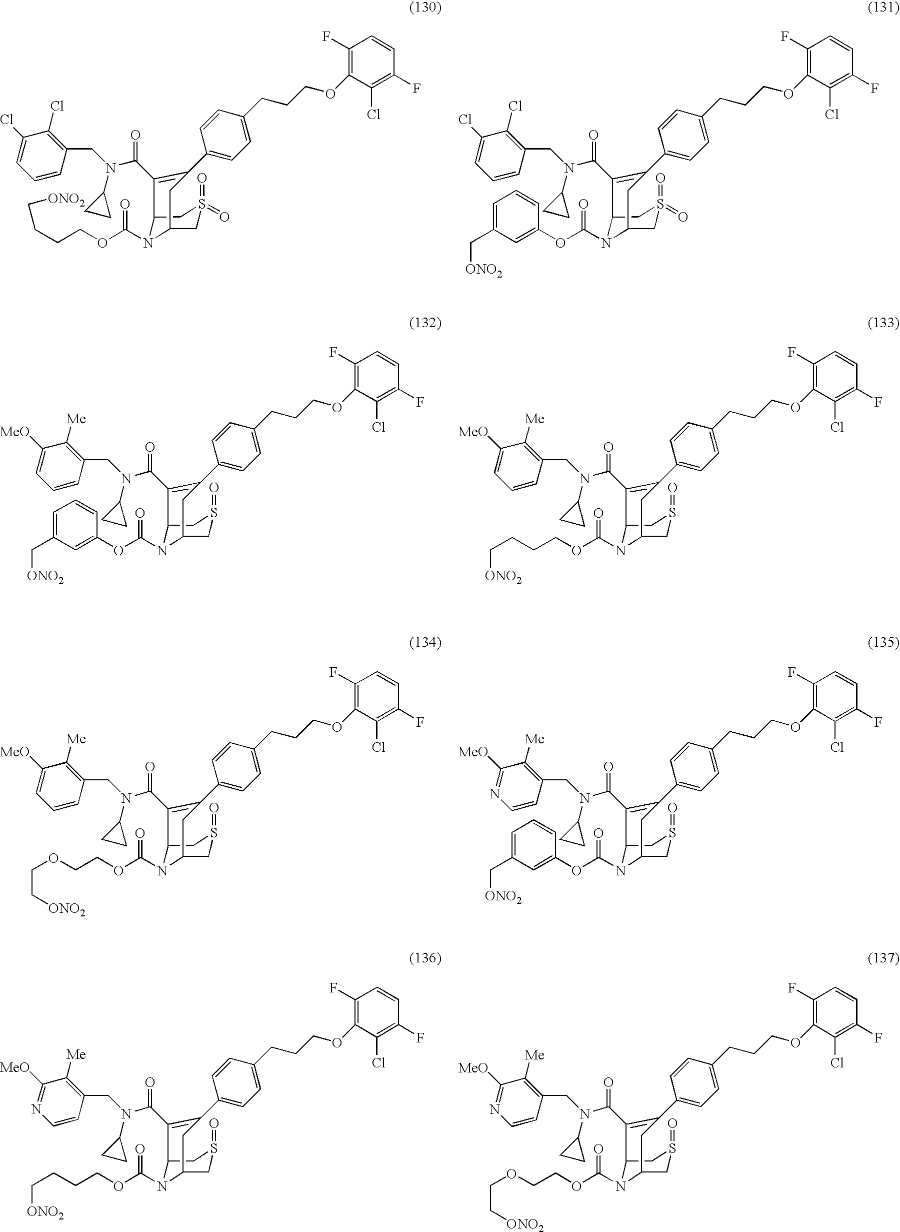

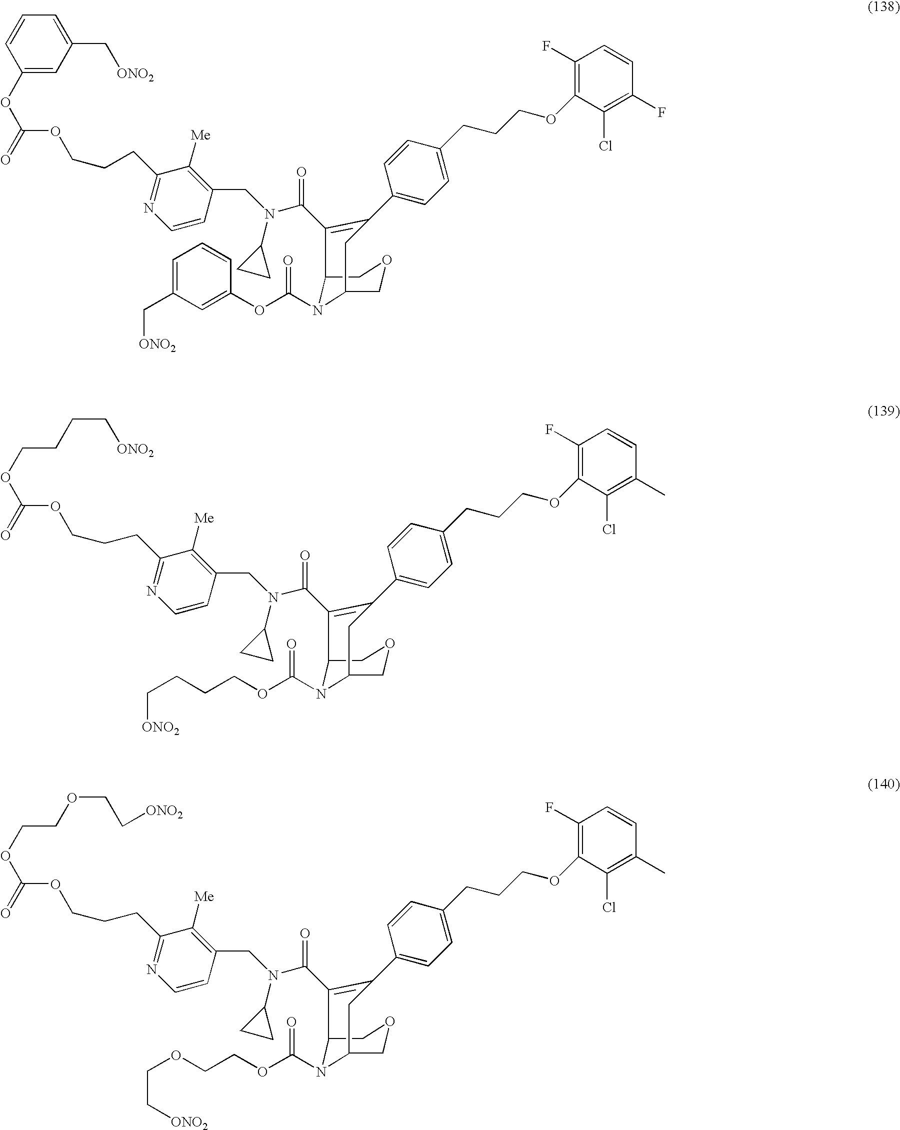

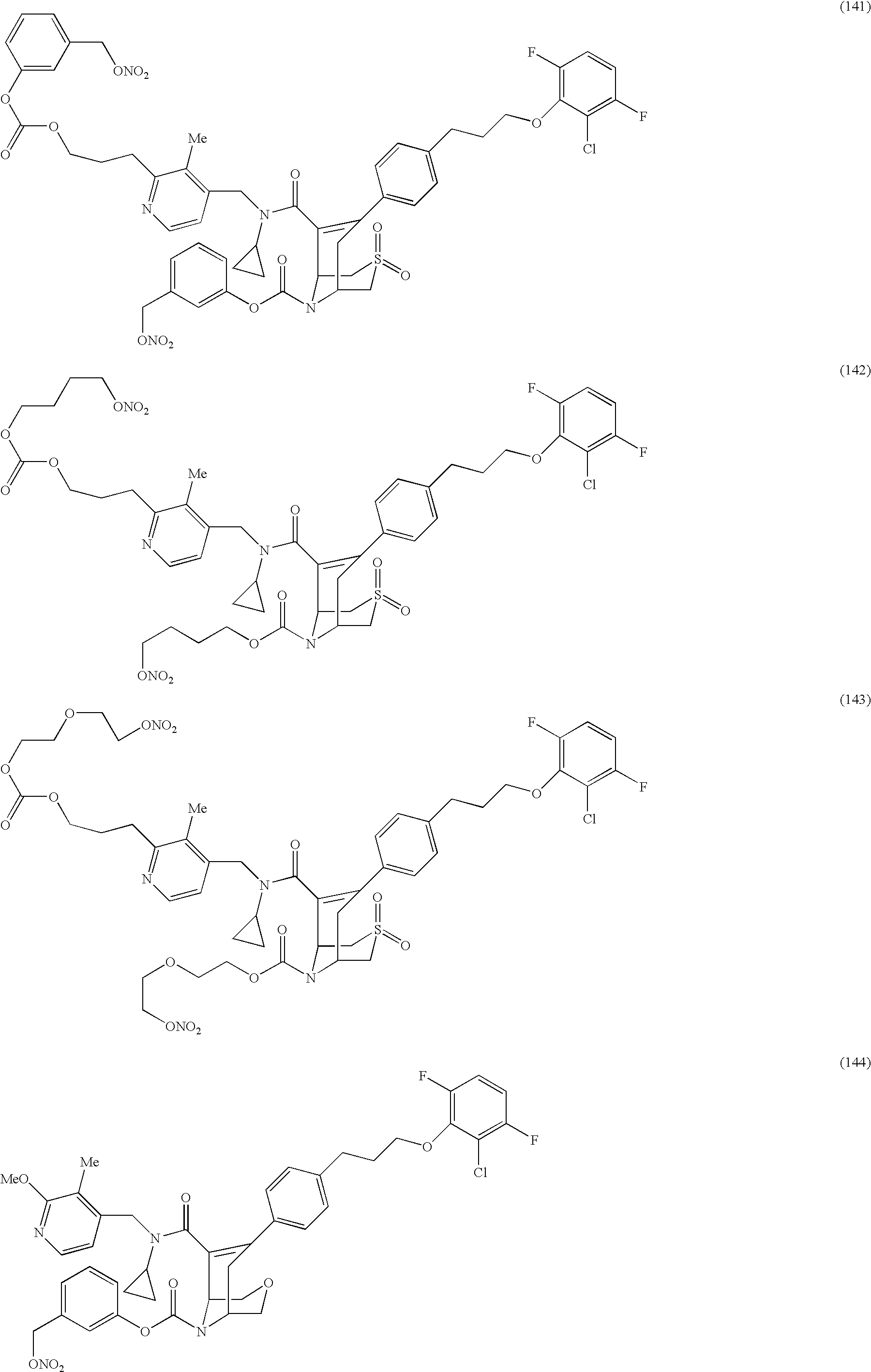

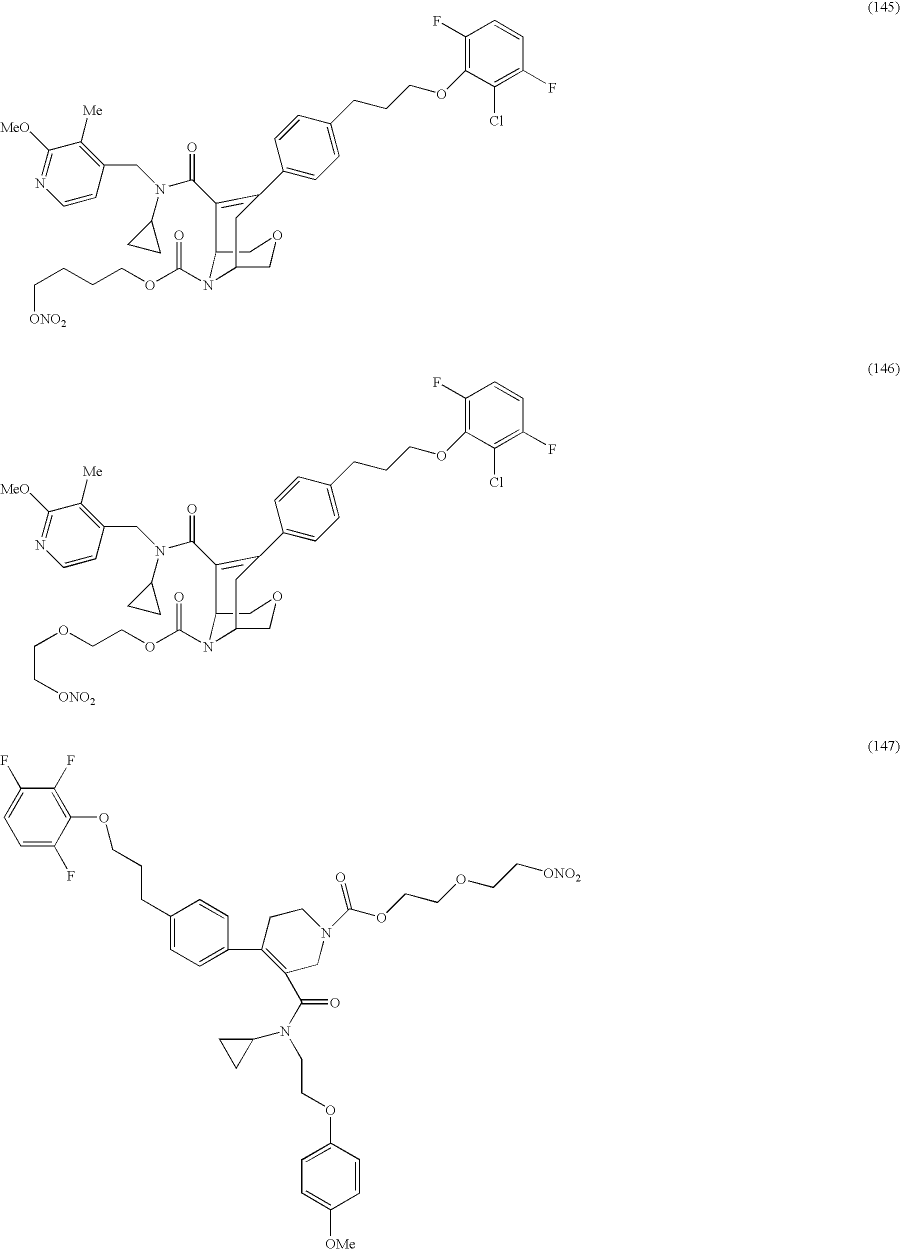

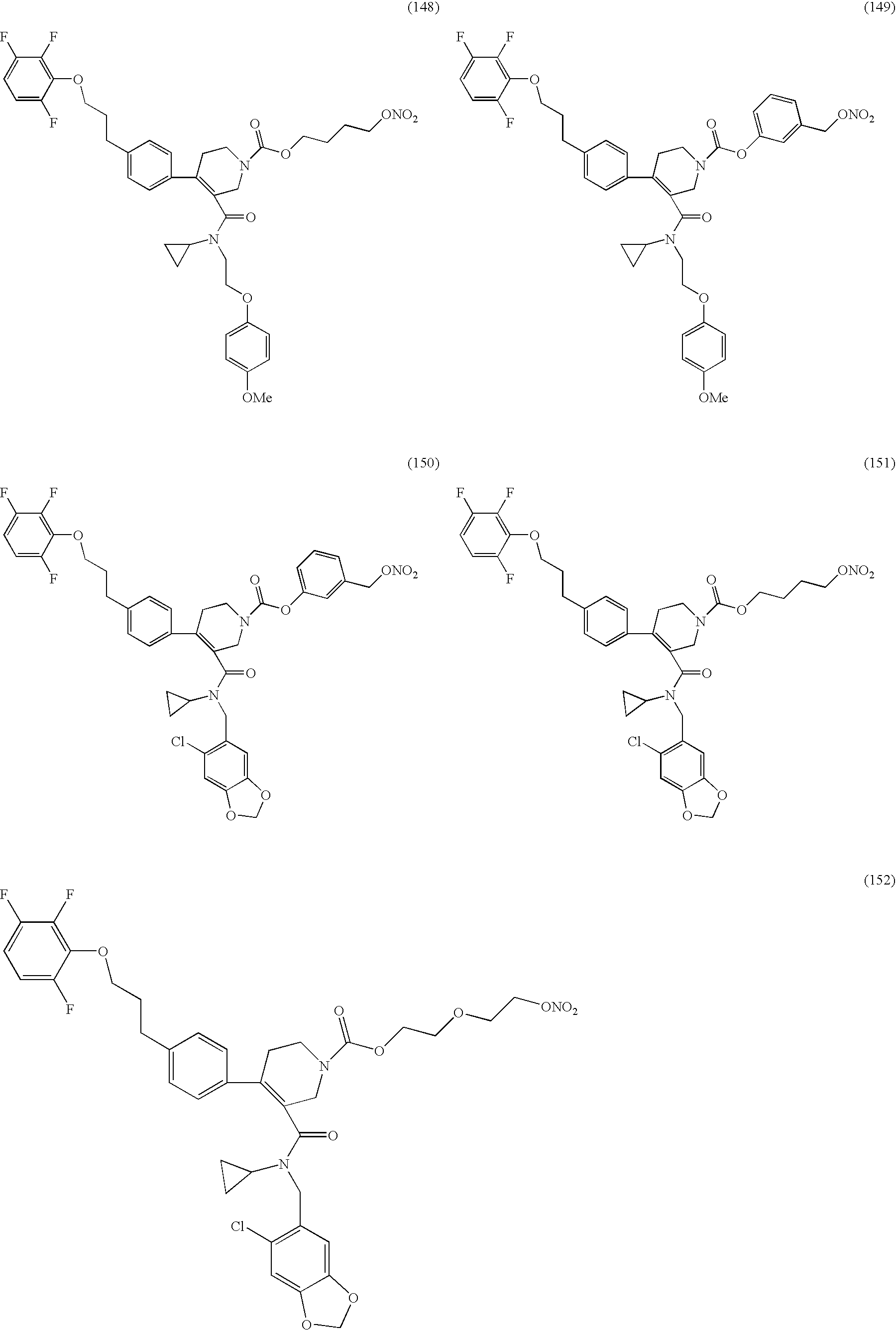

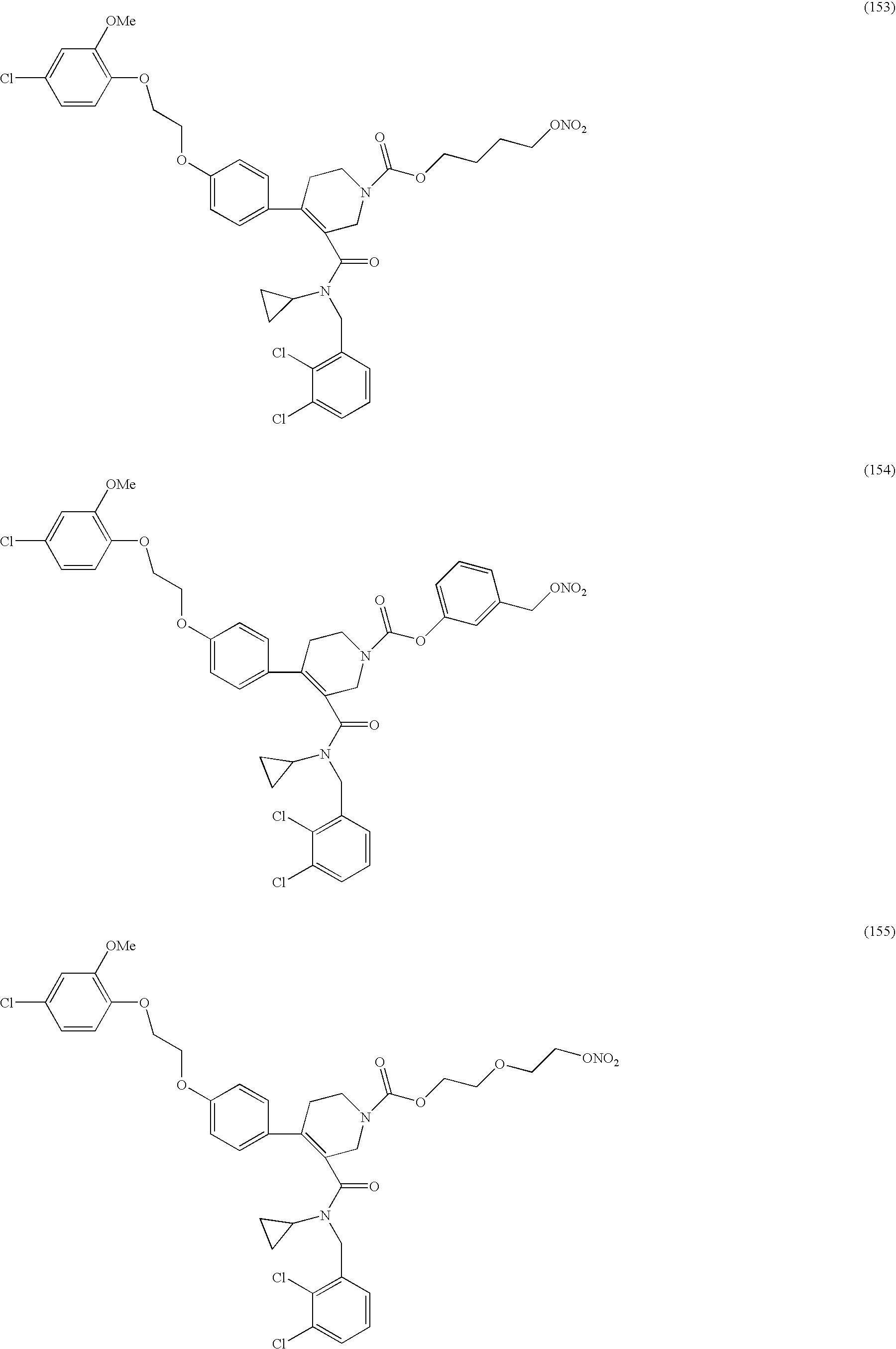

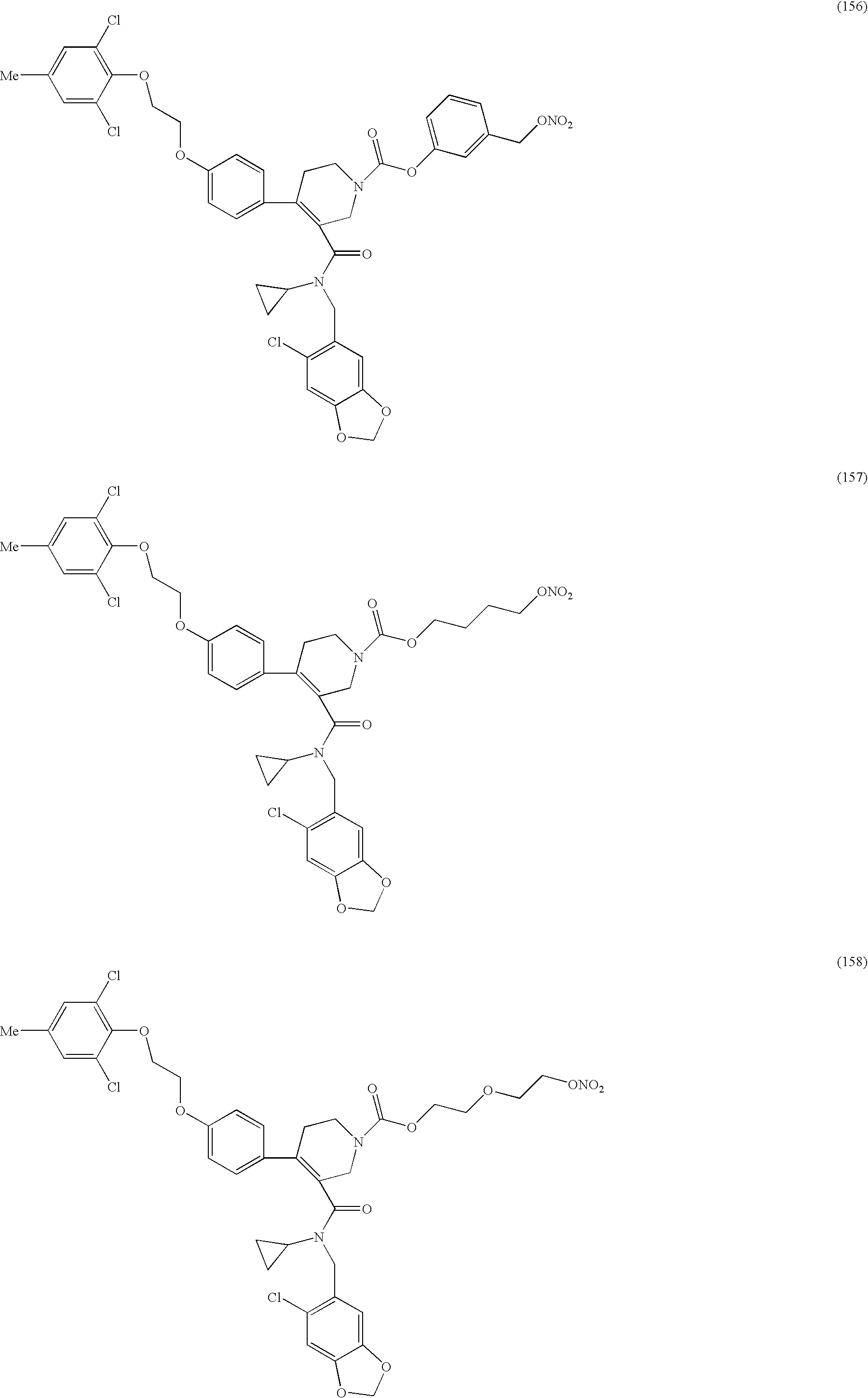

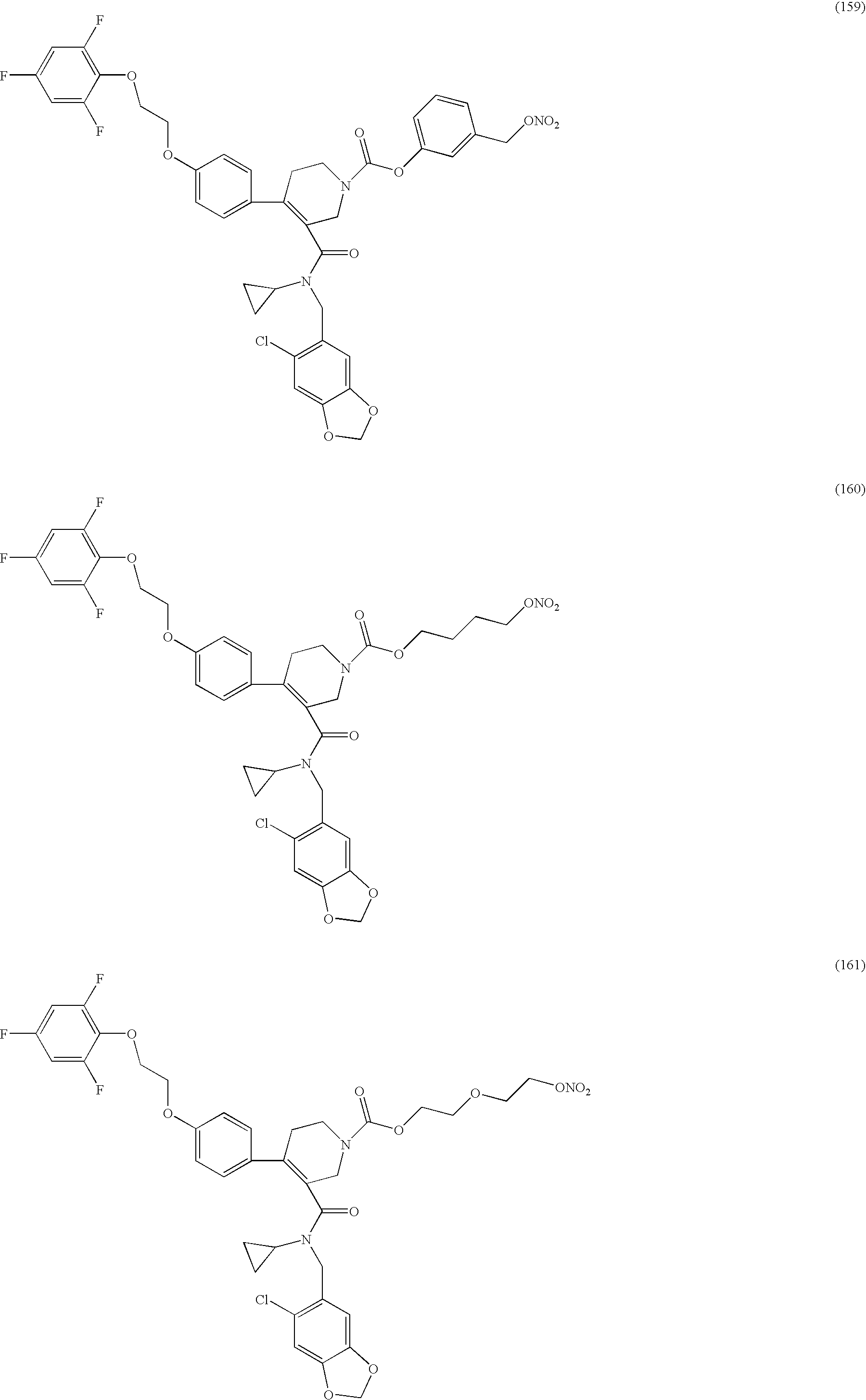

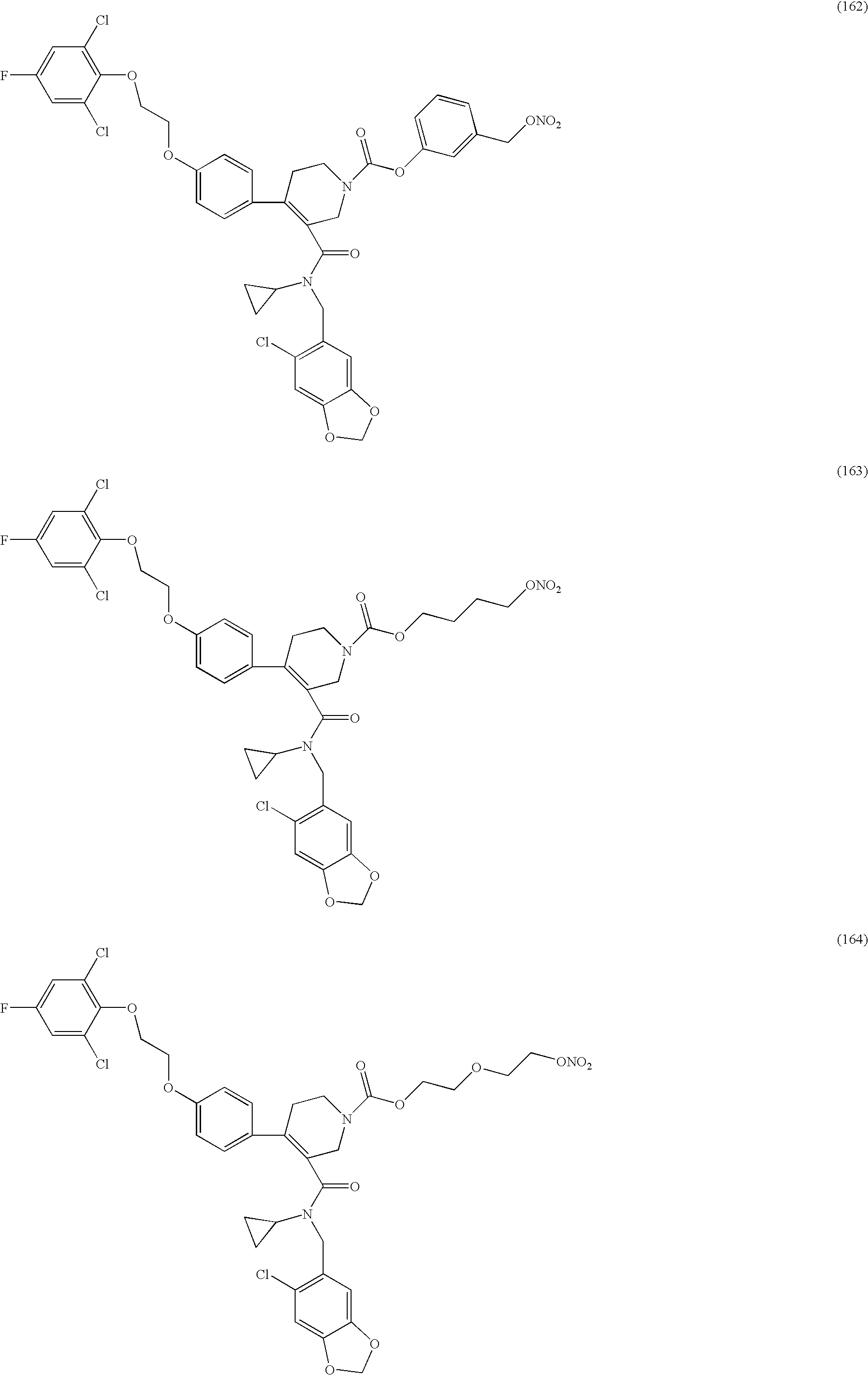

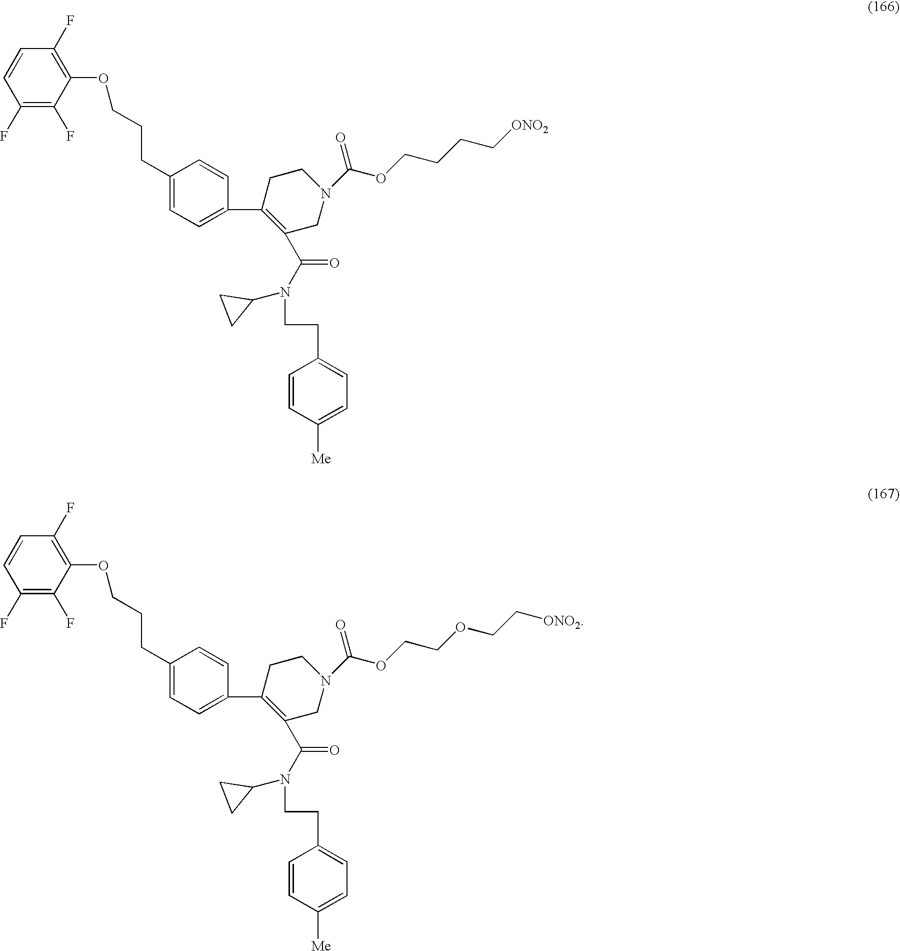

4. A compound of general formula (I) according to claim 1 for use as a medicament.

5. Use of a compound according to claim 1 for preparing a drug having anti-inflammatory, antithrombotic and antiplatelet activity.

6. Use of a compound according to claim 1, for preparing a drug that can be employed in the treatment or prophylaxis of cardiovascular, renal and chronic liver diseases, inflammatory processes and metabolic syndrome.

7. Use of a compound according to claim 6, for preparing a drug that can be employed in the treatment or prophylaxis of congestive heart failure, coronary diseases, left ventricular dysfunction and hypertrophy, cardiac fibrosis, myocardial ischemia, stroke, atherosclerosis, restenosis post angioplasty, renal ischemia, renal failure, renal fibrosis, glomerulonephritis, renal colic, ocular and pulmonary hypertension, glaucoma, systemic hypertension, diabetic complications such as nephropathy, vasculopathy and neuropathy, peripheral vascular diseases, liver fibrosis, portal hypertension, metabolic syndrome, erectile dysfunction, complications after vascular or cardiac surgery, complications of treatment with immunosuppressive agents after organ transplantation, hyperaldosteronism, lung fibrosis, scleroderma, anxiety, cognitive disorders.

8. A pharmaceutical composition comprising a pharmaceutically acceptable carrier and a pharmaceutically effective amount of a compound of general formula (I) or a salt or stereoisomer thereof according to claim 1.

9. A pharmaceutical composition according to claim 8 in a suitable form for the oral, parenteral, rectal, topic and transdermic administration, by inhalation spray or aerosol or iontophoresis devices.

10. Liquid or solid pharmaceutical composition for oral, parenteral, rectal, topic and transdermic administration or inhalation in the form of tablets, capsules and pills eventually with enteric coating, powders, granules, gels, emulsions, solutions, suspensions, syrups, elixir, injectable forms, suppositories, in transdermal patches or liposomes, containing a compound of formula (I) or a salt or stereoisomer thereof according to claim 1 and a pharmaceutically acceptable carrier.

11. A pharmaceutical composition comprising a compound of general formula (I) according to claim 1, at least a compound used to treat cardiovascular disease and a pharmaceutically acceptable carrier.

12. Pharmaceutical composition according to claim 11 wherein the compound used to treat cardiovascular disease is selected from the group consisting of: aldosterone antagonists, angiotensin II receptor blockers, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin or nitrosated compounds thereof.

13. A pharmaceutical kit comprising a compound of general formula (I) as defined in claim 1, a compound used to treat cardiovascular disease as combined preparation for simultaneous, separated or sequential use for the treatment of cardiovascular disease.

14. A pharmaceutical kit according to claim 13 wherein the compound used to treat cardiovascular disease is selected from the group consisting of: aldosterone antagonists, angiotensin II receptor blockers, ACE inhibitors, HMGCoA reductase inhibitors, beta-adrenergic blockers, alpha-adrenergic antagonists, sympatholytics, calcium channel blockers, endothelin antagonists, neutral endopeptidase inhibitors, potassium activators, diuretics, vasodilators, antithrombotics such as aspirin or nitrosated compounds thereof.
